(19) United States
(12) Patent Application Publication
(10) Pub. No.: US 2013/0011424 A1
(43) Pub. Date: Jan. 10, 2013

(54) POLYEPITOPE CONSTRUCTS AND METHODS FOR THEIR PREPARATION AND USE

(75) Inventors: Amir Maksyutov, Novosibirsk (RU); Denis Antonets, Novosibirsk Region (RU); Anastasia Bakulina, Novosibirsk Region (RU); Rinat Maksyutov, Novosibirsk Region (RU)

(73) Assignees: Timur ARTEMEV, Haslemere (GB); Amir MAKSYUTOV, Novosibirsk (RU)

(21) Appl. No.: 13/583,439

(22) PCT Filed: Mar. 9, 2011
(86) PCT No.: PCT/IB2011/000973

§ 371 Date: Sep. 7, 2012

Related U.S. Application Data
(60) Provisional application No. 61/311,981, filed on Mar. 9, 2010.

(51) Int. Cl. C07K 19/00; A61P 37/04; A61P 35/00; C12N 15/62; A61K 39/00

(52) U.S. Cl. 424/185.1; 536/23.4; 530/350; 424/184.1; 424/277.1

(57) ABSTRACT

The invention relates to immunogenic polyepitope constructs containing CTL and/or Th epitopes and optimized spacer sequences which improve processing and presentation of the epitopes leading to induction of high level of both CD4+ and CD8+ specific T-cell responses and specific types of cytokines, and high level of protection and therapeutic activity.

### FIELD OF THE INVENTION

The invention relates to novel immunogenic polyepitope constructs containing CTL and/or Th epitopes and optimized spacer sequences which improve processing and presentation of the epitopes leading to induction of high level of both CD4+ and CD8+ specific T-cell responses and specific types of cytokines, and high level of protection and therapeutic activity.

### BACKGROUND OF THE INVENTION

Breast cancer is the most common cancer found in women. About 13% women in the US will develop breast cancer during their life. About 30% of such cases are advanced forms of cancer which are characterized by the enhanced expression of HER2 protein by tumor cells (Sequeira S J et al., BMC Cell Biol. 2009, 10:64; Hawthorne V S et al., Mol Cancer Res. 2009, 7(4):592-600). HER2 is a member of the EGF family of receptors which control cell proliferation and survival and which is present in normal cells, but in much lower amounts than in cancer cells. Changes in regulation of activity of HER2 protein lead to suppression of apoptosis and active cell proliferation and can lead to cancer (Alroy I and Yarden Y. 2000 Breast Dis. 11:31-48; Harari D and Yarden Y. 2000 Oncogene 19(53):6102-14; Hudziak R M et al., 1987 PNAS, 84(20):7159-63). HER2 overexpression was also found in some other cancers, e.g. in 80% of metastatic prostate cancers (Mossoba M E et al., 2008, Mol. Ther. 16(3):607-617).

Many research groups are now trying to develop anti-cancer vaccines based on various cancer-specific antigens, including HER2. Development of anti-cancer vaccines is very promising, because cancer antigen-specific CTL response can efficiently destroy cancer cells and the mechanisms of immunological memory prevent re-emergence of cancer. Candidate anti-cancer vaccines that are currently being developed on the basis of HER2 utilize both extracellular and intracellular portions of the protein. Several of these candidate vaccines use a single peptide E75 (HER2 amino acids 369-377) (Gates J D et al., 2009 J Am Coll Surg. 208(2):193-201; Mittendorf E A et al., 2008 Cancer Immunol Immunother. 57(10):1511-21; Mittendorf E A et al., 2006 Ann Surg Oncol. 13(8):1085-98), others use several different peptides derived from HER2 (Matsueda S et al., 2009 Anticancer Res 29(7):2427-35; Li Y et al., 2009 Anticancer Res 29(1):41-58; Vertuani S et al., 2009 Cancer Immunol Immunother 58(5):653-64; Scardino A et al., 2007 Cancer Res 67(14):7028-36.) and yet others also contain epitopes from other antigens (Kavanagh B et al. 2007 J Immunother 30(7):762-72). Several candidate vaccines demonstrated induction of humoral immune responses and good safety in primates (Renard V and Leach D R. 2007, Vaccine, 25(2):B17-23). Several CTL-inducing HER2-specific constructs showed low toxicity and lack of autoimmune reactions in clinical studies and also, in some cases, development of both cellular and humoral immune responses (Disis M L et al., 1998, Proc Am Soc Clin Oncol, 17:97a.; Zaks T Z et al., 1998, Cancer Res, 369-377; Knutson K L et al., 2001, J Clin Investig, 107: 477-484; Murray J L et al., 2000, Sem Oncol, 27:71-75; Salazar L G et al., 2003, Clin Cancer Res, 9:5559-5565; Disis M L et al., 2004, J Clin Oncol, 22:1916-1925; Limentani S et al., 2005, ASCO Proc, Abstr 2520).

HER2 peptide E75 (HER2 amino acids 369-377) was shown to be safe and effective in raising a dose-dependent HER2/neu immunity in HLA-A2 and HLA-A3 breast cancer patients (Peoples G E et al., 2005, J Clin Oncol, 23(30):7536-45) and was shown to prevent or delay cancer recurrences (Gates J D et al., 2009, J Am Coll Surg, 208(2):193-201; Peoples G E et al., 2008, Clin Cancer Res, 14(3):797-803; Peoples G E et al., 2005, J Clin Oncol, 23(30):7536-45) and reduce the number of circulating tumor cells (Stojadinovic A et al., 2007, Ann Surg Oncol, 14(12):3359-68). Evaluation of the in vitro immune response of peripheral blood lymphocytes isolated from six consecutive cancer patients immunized with E75 revealed a statistically significant increase in E75-stimulated lymphocytic proliferation. E75-stimulated lymphocytes demonstrated an E75-specific cytolytic response and moreover, these E75-specific lymphocytes also demonstrated tumor-specific lysis against HER2/neu-expressing cancer cell lines (Woll M M et al., 2004, Int J Oncol., 25:1769-1780). E75 vaccination was shown to result in CD4+ recruitment and was associated with a significant decrease in circulating regulatory T cells (Treg) and TGF-β levels (which are primary mediators of immunosuppression leading to tumor survival; see, e.g., Ueda R et al., 2009, Clin Cancer Res, 15(21):6551-6559; Takaku S et al., 2010, Int J Cancer, 126(7):1666-1674) in the majority of the vaccinated patients (Hueman M T et al., 2006, Breast Cancer Res Treat, 98(1):17-29).

Despite some advances described above, there is still no approved vaccine for breast cancer and most other cancers. Thus, there is still a great need for cancer-specific immunogens and vaccines that lead to efficient induction of both CD4+ and CD8+ T cell responses and thus are able to overcome immunosuppression and to provide protective immunity and therapeutic activity.

### SUMMARY OF THE INVENTION

As specified above, there is a great need in the art to develop new immunogenic compositions for efficient induction of immune responses to various clinically relevant antigens. The present invention addresses this and other needs by providing novel polyepitope constructs.

Thus, in one aspect, the invention provides immunogenic polyepitope constructs comprising two or more T cell epitopes selected from the group consisting of:

(SEQ ID NO: 1)
AKFVAAWTLKAAA,
(SEQ ID NO: 7)
AVVGILLVVVLGVVFGILIKRRQQKIR,
(SEQ ID NO: 8)
PICTIDVYMIMVKCWMIDSE,
(SEQ ID NO: 9)
AQMRILKETELRKVKVLGSGA,
(SEQ ID NO: 10)
IKWMALESILRRRFTHQSDV,
(SEQ ID NO: 11)
PICTIDVYMIMVKCWMIDS,
(SEQ ID NO: 21)
CRWGLLLAL,
(SEQ ID NO: 22)
LAALCRWGL,
(SEQ ID NO: 23)
RELGSGLAL,
(SEQ ID NO: 24)
WGLLLALLP,
(SEQ ID NO: 25)
LVVVLGVVF,
(SEQ ID NO: 26)
KITDFGLAR,
(SEQ ID NO: 27)
QLFEDNYAL,
(SEQ ID NO: 28)
YISAWPDSL,
(SEQ ID NO: 29)
GDLTLGLEP,
(SEQ ID NO: 30)
DVWSYGVTV,
(SEQ ID NO: 31)
KIFGSLAFL,
(SEQ ID NO: 32)
FDGDLGMGA,
(SEQ ID NO: 33)
LVHRDLAAR,
(SEQ ID NO: 34)
MELAALCRW,
(SEQ ID NO: 35)
RASPLTSII,
(SEQ ID NO: 36)
RGAPPSTFK,
(SEQ ID NO: 37)
SIISAVVGI,
(SEQ ID NO: 38)
LHCPALVTY,
(SEQ ID NO: 39)
LRIVRGTQL,
(SEQ ID NO: 40)
VKVLGSGAF,
(SEQ ID NO: 41)
LQPEQLQVF,
(SEQ ID NO: 42)
VKIPVAIKV,
(SEQ ID NO: 43)
QLMPYGCLL,
(SEQ ID NO: 44)
QETELVEPL,
(SEQ ID NO: 45)
DIFHKNNQL,
(SEQ ID NO: 46)
ASCVTACPY,
(SEQ ID NO: 47)
TELVEPLTP,
(SEQ ID NO: 48)
PLQRLRIVR,
(SEQ ID NO: 49)
LQVIRGRIL,
(SEQ ID NO: 50)
DEAYVMAGV,
(SEQ ID NO: 51)
EECRVLQGL,
(SEQ ID NO: 52)
TVCAGGCAR,
(SEQ ID NO: 53)
YSEDPTVPL,
(SEQ ID NO: 54)
RWGLLLALL,
(SEQ ID NO: 55)
FEDNYALAV,
(SEQ ID NO: 56)
QEVQGYVLI,
(SEQ ID NO: 57)
LLALLPPGA,
(SEQ ID NO: 58)
GSGAFGTVY,
(SEQ ID NO: 59)
LGISWLGLR,
(SEQ ID NO: 60)
ISAVVGILL,
(SEQ ID NO: 61)
MQIAKGMSY,
(SEQ ID NO: 62)
LSYMPIWKF,
(SEQ ID NO: 63)
GVVKDVFAF,
(SEQ ID NO: 64)
AIKVLRENT,
(SEQ ID NO: 65)
SWLGLRSLR,
(SEQ ID NO: 66)
ILLVVVLGV,
(SEQ ID NO: 67)
FGPEADQCV,
(SEQ ID NO: 68)
TLQGLGISW,
(SEQ ID NO: 69)
TDFGLARLL,
(SEQ ID NO: 70)
DSTFYRSLL,
(SEQ ID NO: 71)
IISAVVGIL,
(SEQ ID NO: 72)
TTPVTGASP,
(SEQ ID NO: 73)
GMEHLREVR,
(SEQ ID NO: 74)
ALCRWGLLL,
(SEQ ID NO: 75)
RIVRGTQLF,
(SEQ ID NO: 76)
GSCTLVCPL,
(SEQ ID NO: 77)
DGENVKIPV,
(SEQ ID NO: 78)
MELAALCRWGLLLALLPPGA,
(SEQ ID NO: 56)
QEVQGYVLI,
(SEQ ID NO: 79)
PLQRLRIVRGTQLFEDNYALAV,
(SEQ ID NO: 72)
TTPVTGASP,
(SEQ ID NO: 45)
DIFHKNNQL,
(SEQ ID NO: 52)
TVCAGGCAR,
(SEQ ID NO: 38)
LHCPALVTY,
(SEQ ID NO: 46)
ASCVTACPY,
(SEQ ID NO: 76)
GSCTLVCPL,
(SEQ ID NO: 73)
GMEHLREVR,
(SEQ ID NO: 31)
KIFGSLAFL,
(SEQ ID NO: 41)
LQPEQLQVF,
(SEQ ID NO: 28)
YISAWPDSL,
(SEQ ID NO: 49)
LQVIRGRIL,
(SEQ ID NO: 80)
TLQGLGISWLGLRSLRELGSGLAL,
(SEQ ID NO: 51)
EECRVLQGL,
(SEQ ID NO: 67)
FGPEADQCV,
(SEQ ID NO: 62)
LSYMPIWKF,
(SEQ ID NO: 81)
RASPLTSIISAVVGILLVVVLGVVF,
(SEQ ID NO: 82)
QETELVEPLTP,
(SEQ ID NO: 83)
VKVLGSGAFGTVY,
(SEQ ID NO: 84)
DGENVKIPVAIKVLRENT,
(SEQ ID NO: 50)
DEAYVMAGV,
(SEQ ID NO: 43)
QLMPYGCLL,
(SEQ ID NO: 61)
MQIAKGMSY,
(SEQ ID NO: 33)
LVHRDLAAR,
(SEQ ID NO: 85)
KITDFGLARLL,
(SEQ ID NO: 30)
DVWSYGVTV,
(SEQ ID NO: 70)
DSTFYRSLL,
(SEQ ID NO: 29)
GDLTLGLEP,
(SEQ ID NO: 32)
FDGDLGMGA,
(SEQ ID NO: 53)
YSEDPTVPL,
(SEQ ID NO: 63)
GVVKDVFAF,
(SEQ ID NO: 36)
RGAPPSTFK,
(SEQ ID NO: 437)
LRHLYQGCQ,
(SEQ ID NO: 39)
LRIVRGTQL,
(SEQ ID NO: 438)
CLHFNHSGICELHCPALV,
(SEQ ID NO: 439)
LQVFETLEE,
(SEQ ID NO: 440)
LRSLRELGS,
(SEQ ID NO: 441)
LCFVHTVPWDQ,
(SEQ ID NO: 442)
LRGQECVEE,
(SEQ ID NO: 443)
CPINCTHSC,
(SEQ ID NO: 444)
IRKYTMRRL,
(SEQ ID NO: 445)
MRILKETELRKVKVLGS,
(SEQ ID NO: 446)
VKIPVAIKVLRENTSPK,
(SEQ ID NO: 447)
YVMAGVGSPYVSRLLGICLTSTVQLV,
(SEQ ID NO: 448)
VRLVHRDLA,
(SEQ ID NO: 449)
FGLARLLDIDETEYH,
(SEQ ID NO: 450)
WMALESILRRRFTHQS,
(SEQ ID NO: 451)
CTIDVYMIMVKCWMI,
(SEQ ID NO: 452)
CRPRFRELVSEFS,
and
(SEQ ID NO: 359)
FVVIQNEDL.

In one embodiment, the epitopes within the polyepitope constructs of the invention are connected end-to-end and/or are connected using spacer sequences which provide optimal processing and presentation of epitopes. In a specific embodiment, such spacer sequences are selected from the group consisting of K/R-K/R, A, AR, ARY, [ANRK][RQYW][YWFVI] (SEQ ID NO: 464), ADLVKV (SEQ ID NO: 2), ADLVAG (SEQ ID NO: 3), ADLAVK (SEQ ID NO: 4), AD, ADL, ADLV (SEQ ID NO: 5), ADLVK (SEQ ID NO: 6), [APRS][DILT][AGL][AKV] (SEQ ID NO: 460), [ARSPNK][DLITGV][LGAVEK][VKAFSI][ALKSEI][GVKLSE] (SEQ ID NO: 461), and [AGNRKP][DIATVG][LGANVE][ASNVLK][VIKAGP][KAGVSE] (SEQ ID NO: 462).

In one embodiment, the polyepitope constructs of the invention further comprise one or more homologous or heterologous targeting signals which direct intracellular transport of the construct to a specific cellular compartment. In a specific embodiment, at least one of said targeting signals is selected from the group consisting of (i) a signal peptide of HER2 protein or a modified version thereof, (ii) an N-terminal portion or the whole sequence of the invariant chain associated with MHC class II molecules, (iii) a C-terminal portion of the human LAMP-1 protein, and (iv) the tyrosine-motif Y-X-X-hydrophobic amino acid, wherein X is any amino acid. In another specific embodiment, at least one of said targeting signals is selected from the group consisting of MELAALCRWGLLLALLPPGAP (SEQ ID NO: 13), MELAALCRWGLLLALLPPGAAS (SEQ ID NO: 14), RKRSHAGYQTI (SEQ ID NO: 15), IPIAVGGALAGLVLIVLIAYLVGRKRSHAGYQTI (SEQ ID NO: 16), LRMKLPKPPKPVSQMR (SEQ ID NO: 17), LRMKLPK (SEQ ID NO: 18), LRMK (SEQ ID NO: 19), and MHRRRSRSCREDQKPVMDDQRDLISNNEQLPMLGRRPGAPESKCSRGALYTGFSILVTLLL AGQATTAYFLYQQQGRLDKLTVTSQNLQLENLRMKLPKPPKPVSKMRMATPLLMQALPM GALPQGPMQNATKYGNMTEDHVMHLLQNADPLKVYPPLKGSFPENLRHLKNTMETIDW KVFESWMHHWLLFEMSRHSLEQKPTDAPPKVLTKCQEEVSHIPAVHPGSFRPKCDENGNY LPLQCYGSIGYCWCVFPNGTEVPNTRSRGHHNCSESLELEDPSSGLGVTKQDLGPVPM (SEQ ID NO: 454)

In one embodiment, the polyepitope constructs of the invention further comprise N-terminally conjugated ubiquitin. In a specific embodiment, the ubiquitin is UbV76 having the sequence MQIFVKTLTGKTITLEVEPSDTIENVKAKIQDKEGIPPDQQRLIFAGKQLEDGRTLSDYNIQK ESTLHLVLRLRGV (SEQ ID NO: 455). In one embodiment, the ubiquitin is conjugated directly to the N terminus of the polyepitope construct. In another embodiment, Arg or Val is inserted between the ubiquitin and the N terminus of the polyepitope construct.

In one embodiment, the polyepitope constructs of the invention comprise the sequence selected from the group consisting of:

(SEQ ID NO: 86)
CRWGLLLALLVVVLGVVFSIISAVVGIRELGSGLALMELAALCRWADLARDEAYVMAGVADLVEECRVLQGLADYSEDPTVPLAVKIPV
AIKVAQLFEDNYALADVWSYGVTVAWGLLLALLPATVCAGGCARADIFHKNNQLADASCVTACPYADLLHCPALVTYATELVEPDTPAD
LKITDFGLARARGAPPSTFKADLYISAWPDSLAQETELVEPLALQVIRGRILALAALCRWGLADLQLMPYGCLLADKIFGSLAFLARGD
LTLGLEPAVKVLGSGAFADLVHRDLAARADLQPEQLQVFADAFDGDLGMGAAPLQRLRIVRADLRIVRGTQLARASPLTSII;
(SEQ ID NO: 87)
QETELVEPLASCVTACPYADLVKVCRWGLLLALSIISAVVGIAARDEAYVMAGVADLVKLHCPALVTYARASPLTSIIADLVEECRVLQ
GLAFDGDLGMGAARGAPPSTFKADLKIFGSLAFLMELAALCRWADLVQLMPYGCLLAQLFEDNYALKITDFGLARADYISAWPDSLTVC
AGGCARADLWGLLLALLPADLVHRDLAARADLYSEDPTVPLRELGSGLALARGDLTLGLEPAVKVLGSGAFADLQPEQLQVFADLDVWS
YGVTVADLRIVRGTQLAPLQRLRIVRADLAALCRWGLAVKIPVAIKVADLQVIRGRILALVVVLGVVFADIFHKNNQLATELVEPLTP;
(SEQ ID NO: 88)
CRWGLLLALASCVTACPYADLYISAWPDSLAVKIPVAIKVAQLFEDNYALADVWSYGVTVAWGLLLALLPADIFHKNNQLATELVEPLT
PADLLHCPALVTYAPLQRLRIVRADLQLMPYGCLLADKIFGSLAFLMELAALCRWADLVHRDLAARADLQPEQLQVFADAFDGDLGMGA
ALQVIRGRILAVKVLGSGAFADLRIVRGTQLARGAPPSTFKADLQETELVEPLRELGSGLALLVVVLGVVFSIISAVVGIARGDLTLGL
EPADKITDFGLARALAALCRWGLADYSEDPTVPLTVCAGGCARARASPLTSIIADLVEECRVLQGLAARDEAYVMAGV;
(SEQ ID NO: 89)
CRWGLLLALAFGPEADQCVADLQLMPYGCLLADYSEDPTVPLAVKIPVAIKVAQLFEDNYALADVWSYGVTVAWGLLLALLPATVCAGG
CARAISAVVGILLATLQGLGISWADSWLGLRSLRADLVKRWGLLLALLLLALLPPGARELGSGLALLVVVLGVVFSIISAVVGIILLVV
VLGVAIISAVVGILAIKVLRENTADLVQETELVEPLALQVIRGRILAGVVKDVFAFADLARDEAYVMAGVADLPLQRLRIVRADLKITD
FGLARALGISWLGLRADLQEVQGYVLIADLHCPALVTYAVKVLGSGAFADGMEHLREVRADTTPVTGASPADASCVTACPYADLYISAW
PDSLARGDLTLGLEPADRGAPPSTFKADLRIVRGTQLATELVEPLTPADAFDGDLGMGAALAALCRWGLADLQPEQLQVFADAFEDNYA
LAVAMQIAKGMSYATDFGLARLLMELAALCRWADLVHRDLAARADGSGAFGTVYARDGENVKIPVADLVDSTFYRSLLADLVEECRVLQ
GLADKIFGSLAFLALCRWGLLLADIFHKNNQLADLSYMPIWKFADLVGSCTLVCPLARASPLTSIIADLRIVRGTQLF;
(SEQ ID NO: 90)
TTPVTGASPADLSWLGLRSLRADLVGSCTLVCPLAIKVLRENTADYSEDPTVPLMELAALCRWADLRWGLLLALLILLVVVLGVADLWG
LLLALLPADLVHRDLAARADLDVWSYGVTVADLGISWLGLRADLVKVQETELVEPLTDFGLARLLRELGSGLALAIISAVVGILAFGPE
ADQCVADLVKVCRWGLLLALISAVVGILLGSGAFGTVYADLSYMPIWKFADLVEECRVLQGLGVVKDVFAFADLAFEDNYALAVADLKI
FGSLAFLASCVTACPYADLVKVQLMPYGCLLAARDEAYVMAGVADLVKLHCPALVTYAVKVLGSGAFADLQPEQLQVFADLRIVRGTQL
FADLVDSTFYRSLLADGMEHLREVRADLRIVRGTQLATVCAGGCARADLAALCRWGLAPLQRLRIVRADLQVIRGRILALVVVLGVVFA
DIFHKNNQLATLQGLGISWAQLFEDNYALARGDLTLGLEPAARDGENVKIPVADLVALCRWGLLLALLALLPPGAARGAPPSTFKADLK
ITDFGLARADMQIAKGMSYADAFDGDLGMGAAVKIPVAIKVARASPLTSIIADLQEVQGYVLIADYISAWPDSLSIISAVVGIATELVE
PLTP;
(SEQ ID NO: 91)
CRWGLLLALISAVVGILLAFGPEADQCVADLQETELVEPLTDFGLARLLRELGSGLALLVVVLGVVFSIISAVVGIILLVVVLGVAIIS
AVVGILGSGAFGTVYAIKVLRENTADLRIVRGTQLFADLVKLHCPALVIYAVKVLGSGAFADGMEHLREVRADYISAWPDSLALCRWGL
LLAVKIPVAIKVALAALCRWGLADTTPVTGASPADRGAPPSTFKADLYSEDPTVPLAFDGDLGMGALLALLPPGAARDGENVKIPVADL
VDSTFYRSLLADGSCTLVCPLMELAALCRWADSWLGLRSLRADLVPLQRLRIVRADLKITDFGLARALGISWLGLRADLQEVQGYVLIA
DKIFGSLAFLASCVTACPYADLRASPLTSIIADLVEECRVLQGLAARDEAYVMAGVADLRWGLLLALLGVVKDVFAFADLQLMPYGCLL
ADLQPEQLQVFADLRIVRGTQLAMQIAKGMSYADVWSYGVTVAWGLLLALLPATVCAGGCARAQLFEDNYALARGDLTLGLEPADIFHK
NNQLATELVEPLTPADLVHRDLAARADAFEDNYALAVALQVIRGRILATLQGLGISWADLSYMPIWKF;
(SEQ ID NO: 92)
TVCAGGCARADGMEHLREVRADGKEECRVLQGLADGRELGSGLALPQLFEDNYALSDGQETELVEPLPLVVVLGVVFARDGENVKIPVA
LLALLPPGAAQEVQGYVLIPDLARGDLTLGLEPAIKVLRENTADAFDGDLGMGAPDAKARDEAYVMAGVADIFHKNNQLAVKVLGSGAF
ATLQGLGISWAIAFGPEADQCVPDLKLSYMPIWKFADLKPLQRLRIVRAIISAVVGILMELAALCRWATGVVKDVFAFADLVKIPVAIK
VSIISAVVGIPISAVVGILLPILQPEQLQVFADGKYSEDPTVPLADMQIAKGMSYARGAPPSTFKADLQVIRGRILPDGRASPLTSIIA
DLVHRDLAARADSWLGLRSLRADGKLGISWLGLRADGVKITDFGLARATDFGLARLLPDGDSTFYRSLLAILLVVVLGVADTTPVTGAS
PRDLRIVRGTQLATELVEPLTPPDLKASCVTACPYPILAALCRWGLADAFEDNYALAVAIDVWSYGVTVAWGLLLALLPRDAKQLMPYG
CLLAIKIFGSLAFLALCRWGLLLRDGRIVRGTQLFADLVGSGAFGTVYADGGSCTLVCPLPDGYISAWPDSLRDLHCPALVTYALLVCR
WGLLLALRWGLLLALL;
(SEQ ID NO: 93)
MELAALCRWGLLLALLPPGAPDGENVKIPVAIKVLRENTADGKEECRVLQGLPDGKYSEDPTVPLPDDEAYVMAGVADLKQETELVEPL
TPPDGRASPLTSIISAVVGILLVVVLGVVFPDAGMEHLREVRADGKDIFHKNNQLPDLQPEQLQVFRDAQEVQGYVLIPDLAFDGDLGM
GAPDLQVIRGRILPDVKVLGSGAFGTVYPIGDLTLGLEPPDLKASCVTACPYATLQGLGISWLGLRSLRELGSGLALPMQIAKGMSYAL
FGPEADQCVPDLKLSYMPIWKFADLKPLQRLRIVRGTQLFEDNYALAVARGAPPSTFKAGVVKDVFAFRDLVKITDFGLARLLPLVHRD
LAARADVWSYGVTVRDTTPVTGASPRDLYISAWPDSLRTVCAGGCARSDKIFGSLAFLPDLHCPALVTYADDSTFYRSLLADGKQLMPY
GCLLADGGSCTLVCPL;
(SEQ ID NO: 110)
WGLLLALLP-RDA-YSEDPTVPL--ADIDETEYHA-PDLK-AREEGAGSDVFD--AYGVTVWELM-ALGK-ARDDDDMGDLVD-PLGK-
AEITGYLYIS-ADGK-HLDMLRHLY-ADLK-AHSDCLACLH-AD-LTCSPQPEY-ADLK-QSDVWSYGV-AD-AYKDPPFCVA-PDL-
ARDGDLGMGAA-PIAK-LLDIDETEY-AD-ARDGDPASNTA-AI-ARDGENVKIPV-ALL-GSGAFGTVY-PD-NASLSFLQD-PLLK-
LHCPALVTY-AD-DSTFYRSLL-ADL-FSPAFDNLY-AILK-TIDVYMIMV;
(SEQ ID NO: 123)
TIDVYMIMV-PDLK-CRWGLLLAL-A-LLALLPPGA-ADG-AILDEAYVMA--ALIHHNTHL-PDL-RLVHRDLAA--LLLALLPPG-
ADGK-QLFEDNYAL-P-ILHNGAYSL-P-SLTLQGLGI-R-LVDAEEYLV-R-ILLVVVLGV-ADA-SIISAVVGI-A-RLLQETELV-
AD-AFEDNYALAV--AVVGILLVV-A-VVLGVVFGI-AD-ALLNWCMQIA-ADLV-ALCRWGLLL-AD-YISAWPDSL-RD-
KIFGSLAFL-RDL-QLMPYGCLL-ADG-MIMVKCWMI;
(SEQ ID NO: 124)
MELAALCRWGLLLALLPPGAPPDLLALLPPGAPDATLEEITGYLAILDEAYVMAPILHNGAYSLPQLFEDNYALSIISAVVGIAQLMPY
GCLLRLLVVVLGVVRDLQLRSLTEIAILLVVVLGVPDAVVGILLVVADALCRWGLLLADYISAWPDSLRDKIFGSLAFL;
(SEQ ID NO: 138)
LVPQQGFFC-ADLV-PCARVCYGL-PDLK-KHSDCLACL--ATLEEITGYL-A-TLSPGKNGV-PDL-DLVDAEEYL-P-
ILHNGAYSL-A-SLPDLSVFQ-RD-QIAKGMSYL--AILDEAYVMA--ALIHHNTHL-AI-AFGPEADQCV-RDLK-LVDAEEYLV-A-
QLFEDNYAL--SIISAVVGI-ADG-THLDMLRHL--ACLTSTVQLV-ADG-FRNPHQALL-ADG-RLLQETELV-ADL-KIFGSLAFL-
A-YISAWPDSL-RD-AYSLTLQGL-RDL-TYLPTNASL-SDA-RWGLLLALL-A-QLMPYGCLL-ADG-MIMVKCWMI;
(SEQ ID NO: 148)
HYKDPPFCV-AIGK-AIQNEDLGPA-RDL-QIAKGMSYL-A-TLSPGKNGV-SD-LLALLPPGA-ADG-PYVSRLLGI--
AYLSTDVGSC-AD-ILLVVVLGV-ADA-SIISAVVGI-AD-SLRELGSGL-PTG-RASPLTSII-A-LLVVVLGVV-RDL-
AYLTPQGGAA--ALIHHNTHL-AD-ARPLTSIISAV-ADL-FRNPHQALL-ADGK-KIFGSLAFL--ALLNWCMQIA-ADLK-
ACLTSTVQLV-ADG-YISAWPDSL-A-HLYQGCQVV-ADL-SLTLQGLGI-AD-QLMPYGCLL-ADG-MIMVKCWMI;
(SEQ ID NO: 156)
CRWGLLLAL-PD-AIQNEDLGPA--AVLDNGDPL--RLLQETELV-ADG-FRNPHQALL-PDLK-QVFETLEEI-PD-QIAKGMSYL-
PD-VVLGVVFGI-ADA-TQLFEDNYA-AD-AVVGILLVV-AD-RASPLTSII-A-LLVVVLGVV-RD-LQLRSLTEI-A-
ILLVVVLGV-ADA-SIISAVVGI-PD-YVLIAHNQV-AD-VKIPVAIKV--ALIHHNTHL-A-LAALCRWGL-A-SAVVGILLV-
ADGK-KIFGSLAFL-A-IWIPDGENV-AD-TIDVYMIMV-QLMPYGCLL-ADG-MIMVKCWMI;
(SEQ ID NO: 183)
CVNCSQFLR-AD-LVKSPNHVK-A-ILKETELRK-RDLK-ARILHNGAYS-AD-GVVFGILIK-ADG-AELMTFGAKP-PDGK-
LELTYLPTN-ALGK-KIRKYTMRR-ADLV-LERPKTLSP-A-VLRENTSPK-A-LLLALLPPG-ADGK-RSLTEILKG--
ALLHTANRP-A-ILIKRRQQK-ADGK-AGILLVVVLG-PDGK-TVWELMTFG-A-ILWKDIFHK-ADGK-RGAPPSTFK-ADL-
QLVTQLMPY-A-VVVLGVVFG-PD-VMAGVGSPY-AILK-LAARNVLVK-ADL-YTMRRLLQE-ADGK-TFYRSLLED-RD-
VVFGILIKR-A-LAFLPESFD-A-YLYISAWPD-AD-MTFGAKPYD;
(SEQ ID NO: 194)
RWGLLLALL-A-EYVNARHCL-R-DLLEKGERL--AEYHADGGKV-S-DIFHKNNQL-A-QLFEDNYAL-P-LAALCRWGL-AI-
AYGVTVWELM-AI-LRIVRGTQL--ILLVVVLGV-ADA-TYLPTNASL-A-IWIPDGENV-RLL-VWSYGVTVW-AL-EYLVPQQGF-
ADLK-DVWSYGVTV-PDLK-RFRELVSEF-PDLK-LSYMPIWKF-ADL-SYGVTVWEL-ADA-QCVNCSQFL-ADAK-VYMIMVKCW-
AILK-KWMALESIL-AI-MIMVKCWMI;
(SEQ ID NO: 197)
AWPDSLPDL--DLLEKGERL-RDG-PYVSRLLGI-PDL-TLQGLGISW-A-SLAFLPESF-PDGK-AVVGILLVV-RT-LVVVLGVVF-
A-IWIPDGENV-RLL-VWSYGVTVW-AL-EYLVPQQGF-ADLK-QLMPYGCLL-AD-SYGVTVWEL-ADL-TYLPTNASL-A-
RIVRGTQLF-RWGLLLALL-A-KWMALESIL-AIGV-VYMIMVKCW;
(SEQ ID NO: 211)
RMARDPQRF-AD-AVRGTQLFED-RD-LQPEQLQVF-ADG-EYVNARHCL-ADA-RWGLLLALL--ASEGAGSDVF--AGEGLACHQL-
PDLK-LQGLGISWL-AI-SYGVTVWEL-AD-AWPDSLPDL-PL-EYLVPQQGF-ADGK-HNGAYSLTL--AFNHSGICEL-A-
YLVPQQGFF-ADGV-AYSLTLQGL-PDLK-RFRELVSEF-ADGK-ACYGLGMEHL-AL-VWSYGVTVW-AI-AFQNLQVIRG-ADG-
VTVWELMTF-ADGK-AFYRSLLEDD-RDL-TYLPTNASL-AI-VYMIMVKCW-AILK-KWMALESIL-AD-RFTHQSDVW;
(SEQ ID NO: 224)
CTIDVYMIM-PI-ICELHCPAL-A-QLVTQLMPY-ADG-VSRLLGICL--ALCRWGLLL-PDLK-ARDEAYVMAGV-AD-
ETLEEITGY-A-TEILKGGVL-P-QLFEDNYAL-PD-LQPEQLQVF-AD-KVPIKWMAL--SIISAVVGI-RD-DTILWKDIF-ALGV-
AETHLDMLRH-A-DVFDGDLGM-PDLK-SLRELGSGL--STVQLVTQL-PLGK-ISWLGLRSL--AFDGDLGMGA-AD-CRWGLLLAL-
PD-VTVWELMTF-ADGK-AFEDNYALAV-RDLK-HTVPWDQLF;
(SEQ ID NO: 239)
LHCPALVTY-SD-LTCSPQPEY-ADL-RLVHRDLAA-ALG-HLDMLRHLY-AD-LVVVLGVVF-PDGK-DIFHKNNQL-AD-
LEEITGYLY-AD-GVVKDVFAF-AD-ARPGGLRELQL-AD-ETLEEITGY-ALL-THQSDVWSY-AD-AYLEDVRLVH-PDLK-
QVVQGNLEL-AI-GSGAFGTVY-RL-VMAGVGSPY-AILK-LMTFGAKPY-AD-GTQLFEDNY-ADGK-CVTACPYNY-ADG-
GTVYKGIWI-ADL-SMPNPEGRY-ADLK-HTVPWDQLF-ADLK-SLTLQGLGI-AD-MQIAKGMSY-A-ICLTSTVQL-SD-
DVWSYGVTV-PDLK-MSYLEDVRL-RD-VCTGTDMKL-AD-FSPAFDNLY-AIL-SPAFDNLYY;
(SEQ ID NO: 258)
KIRKYTMRR-A-YLYISAWPD--LVKSPNHVK-PLLK-KVKVLGSGA-PDG-KETELRKVK-PD-AIKVLRENT-AD-GGKVPIKWM-
ADG-NVKIPVAIK-AD-ARGGCLLDHVRE--AGLRSLRELG-ADG-RPKTLSPGK-AI-LQRLRIVRG-PDGV-KLRLPASPE-A-
WGLLLALLP-AD-RSRACHPCS-AILK-KRRQQKIRK-ADLK-HVRENRGRL-AD-ARPGKNGVVKD-A-PLQRLRIVR-RDAK-
AARNVLVKS-AD-MARDPQRFV-A-VLRENTSPK-ADL-VARCPSGVK-ADL-HYKDPPFCV-AD-KIFGSLAFL-A-STFKGTPTA-
ADL-TQRCEKCSK;
(SEQ ID NO: 270)
SMPNPEGRY-ADL-KHSDCLACL--ADMGDLVDAE-RDGK-CVTACPYNY-AL-GGAVENPEY-AL-AVVKDVFAFG-PLAK-
AEIPDLLEKG-PDGK-HLDMLRHLY-ADLK-TVWELMTFG-AD-LTCSPQPEY-ADL-RSSSTRSGG-ADGK-ETLEEITGY-AD-
VLQGLPREY-AD-ARPLISIISAV-AL-ASCVTACPY-PLL-SAVVGILLV-ADLV-AESFDGDPAS-R-DVFDGDLGM-PIL-
AAPRSPLAPS-AI-GTQLFEDNY-AIG-ASLTEILKGG-AD-KGMSYLEDV-AD-VMAGVGSPY-ATLK-SLPDLSVFQ-RDLK-
THQSDVWSY-ADA-SPAFDNLYY-ADL-FSPAFDNLY-ADLK-YYWDQDPPE-ADLV-LMTFGAKPY;
(SEQ ID NO: 285)
QALLHTANR-AIG-RQVPLQRLR-ADGK-QKIRKYTMR-ADGK-GVGSPYVSR--RILKETELR-ADL-LEDVRLVHR-ADG-
TLIDTNRSR-ADL-GMEHLREVR-ADGK-REGPLPAAR-RIG-MALESILRR-PDGK-LGISWLGLR-ADGV-KITDFGLAR-A-
PLQRLRIVR-ADG-VVFGILIKR-RDGK-LVHRDLAAR-A-TVCAGGCAR-RDG-KIRKYTMRR-ADG-AALCRWGLL-ADGK-
KIFGSLAFL-PDG-KVPIKWMAL-SD-ASPLDSTFYR-ADL-VSEFSRMAR-ADLV-CVNCSQFLR-ADLK-LACHQLCAR-AD-
VFQNLQVIR-AIL-SWLGLRSLR;
(SEQ ID NO: 304)
AAPRSPLAPS--ALPAARPAGA-PDG-ALPTHDPSPL-A-ALPASPETHL-SD-ASPETHLDML--AVLDNGDPL--ASPKANKEIL-
P-GAVENPEYL--ASPGKNGVVK-AD-LPTNASLSF--ADPASNTAPL--AARPAGATL--AAPQPHPPPA-ADGV-LQVIRGRIL-
PDG-RASPLTSII-ADL-APPSPREGPL-RDLK-HVRENRGRL-SDL-AHPPPAFSPA-PDLK-AMPNQAQMRI-ADLV-
RKYTMRRLL-A-GVVKDVFAF-AD-AVPLQRLRIV-ADGK-GSCTLVCPL-AI-ASPREGPLPA-ADL-RCEKCSKPC;
(SEQ ID NO: 305)
MELAALCRWGLLLALLPPGAPASPKANKEILAARPAGATLALPTHDPSPLAALPASPETHLSDASPETHLDMLADAPPSPREGPLRDLK
HVRENRGRLADLACPSGVKPDLADGSTRSGGGDLPIASPLTSIISA;
(SEQ ID NO: 319)
YISAWPDSL-PDL-ECRPRFREL-AD-VGILLVVVL-PD-QQKIRKYTM-AD-LFRNPHQAL-AL-LIKRRQQKI-ADLK-
AYGVTVWELM-PDLK-LGMEHLREV--ASPKANKEIL--ALIHHNTHL-A-DIFHKNNQL-AD-MVHHRHRSS-AD-AVPLQRLRIV-
A-ILLVVVLGV-AD-VSRLLGICL--AFGLARLLDI-AI-LQRLRIVRG-AD-VVGILLVVV-PDG-KVPIKWMAL--SLAFLPESF-
AI-LQVIRGRIL--LVVVLGVVF-A-MRILKETEL-RTG-VLIQRNPQL-PDLK-ILRRRFTHQ-AD-LAALCRWGL-AD-
LDSTFYRSL-RD-LRIVRGTQL-PIAK-ISAVVGILL-AI-MIMVKCWMI;
(SEQ ID NO: 320)
MELAALCRWGLLLALLPPGAPAIGFHKNNQLALASPKANKEILRDGKDIFHKNNQLPDGKLGMEHLREVADLFRNPHQALALLGCKKIF
GSLPDLRIVRGTQLADGVMRILKETELSDGQLRSLTEILADGKECRPRFRELADGQLMPYGCLLPDLK;
(SEQ ID NO: 327)
LVVVLGVVF-A-IQRNPQLCY-AILV-TQCVNCSQF-ADG-TLIDTNRSR--ASEGAGSDVF--ALIHHNTHL-AI-AYGVTVWELM-
AIGK-ISWLGLRSL-S-VKVLGSGAF-A-QLFEDNYAL-PLG-RELGSGLAL--ASCVTACPY-AIL-VTSANIQEF-AIG-
VQGNLELTY-AD-LTCSPQPEY-ADLK-QVVQGNLEL-AI-GSGAFGTVY-RL-VMAGVGSPY-ADGV-LQVIRGRIL--
SLAFLPESF-ADG-VWSYGVTVW-ADA-RIVRGTQLF-WCMQIAKGM-AD-MQIAKGMSY-A-LMTFGAKPY-RDL-RACHPCSPM;
(SEQ ID NO: 335)
LRIVRGTQL--ASEGAGSDVF--ALDIDETEYH-ADLK-QETELVEPL-AD-ARPEYLTPQGG-ADGV-EEITGYLYI-PDGK-
EECRVLQGL-ADG-RELGSGLAL--AEDLGPASPL-A-TEILKGGVL-P-LEEITGYLY-PLGK-AGDLGMGAAK-AD-LELTYLPTN-
RDG-VKVLGSGAF-AD-TELVEPLTP-RDLK-SAWPDSLPD-AD-DVWSYGVTV-AD-MQIAKGMSY-AD-QRFVVIQNE;
(SEQ ID NO: 351)
GRILHNGAY-ADG-CRWGLLLAL--LQPEQLQVF--AILDEAYVMA-RD-AKGLQSLPT-AD-GRLGSQDLL-ADG-RELGSGLAL--
AYLEDVRLVH-RD-AFAGCKKIFG-ADG-FRNPHQALL-PIGK-AGEGLACHQL-AD-ARPAGATLE-SL-RRLLQETEL--
AAGCTGPKH-AD-AVRGTQLFED-RDLV-RKYTMRRLL-RD-LRIVRGTQL-PDLK-RNPQLCYQD-ADLK-RQVPLQRLR-ADAK-
ARVCYGLGM-ADGV-HRDLAARNV-PD-QRASPLTSI-PLLK-HRHRSSSTR-ADLV-YLYISAWPD-ADAK-QRFVVIQNE-ADLV-
RRQQKIRKY-ADLK-CRVLQGLPR-ADL-YTMRRLLQE-ADLK-RRFTHQSDV;
(SEQ ID NO: 363)
HTVPWDQLF-ADLV-CRWGLLLAL-RI-ALDIDETEYH-ADL-ARDGDLGMGAA-RD-LPTNASLSF--ADPASNTAPL--
AALPTHDPSPL-AD-NKEILDEAY--ADPAPGAGGM-AI-AEPLTPSGAM-A-GVVKDVFAF-AD-LTCSPQPEY-ADLK-
LVTYNTDTF-AD-LALLPPGAA-PD-EILDEAYVM-P-LVVVLGVVF--AECVGEGLAC-A-TPTAENPEY-AD-RSLLEDDDM-
ALLV-FVVIQNEDL-AL-AMPNQAQMRI-ADLV-MSYLEDVRL-AI-LMTFGAKPY-AD-ICELHCPAL-ALGK-YYWDQDPPE-ADL-
SPAFDNLYY-ADL-FSPAFDNLY-AILK-AMPYGCLLDH;
(SEQ ID NO: 364)
MELAALCRWGLLLALLPPGAPADGKTPTAENPEYAALPASPETHLPILKYSEDPTVPLPDGALPTHDPSPLADNKEILDEAYADEILDE
AYVMPLVVVLGVVFADMQIAKGMSYALMTFGAKPYPLGKAPPPAFSPAFADLHCPALVTY;
(SEQ ID NO: 374)
MELAALCRW-RDLAARNVL-PDA-QETELVEPL--AEEEAPRSPL-PDGK-EECRVLQGL-ADA-GERLPQPPI-ADG-
SETDGYVAP-PDA-AGEGLACHQL-ADG-RELGSGLAL-P-QLFEDNYAL-PD-ALEDDDMGDL-PDLK-REVRAVTSA--
ASEGAGSDVF-A-TEILKGGVL-PL-EEITGYLYI-PDGK-AENPEYLGL-PDLK-QEVQGYVLI-AD-EQLQVFETL-A-
QVVQGNLEL-A-QEFAGCKKI--ALCRWGLLL-RD-AFEDNYALAV;
(SEQ ID NO: 384)
ISWLGLRSL--AEEEAPRSPL--RDLAARNVL-RLG-GENVKIPVA-RLG-KHSDCLACL-AIG-GERLPQPPI-ADL-TGTDMKLRL-
PDGK-AENPEYLGL-ADG-RELGSGLAL--REVRAVTSA-ADG-REYVNARHC-A-QEFAGCKKI-A-QETELVEPL-A-
TELRKVKVL--TDMKLRLPA-ADLK-QEVQGYVLI-PDL-ARGGSRCWGESS-ALGV-KITDFGLAR-A-TDFGLARLL-PDA-
RKYTMRRLL-ADG-RELQLRSLT-ADLK-LDSTFYRSL--MELAALCRW-A-TLQGLGISW-ADL-CQSLTRTVC-ALL-
HYKDPPFCV-AIG-YISAWPDSL-AD-CRWGLLLAL-RDL-TRTVCAGGC-ADLK-TFYRSLLED;
(SEQ ID NO: 389)
TRTVCAGGC-ADG-GGGDLTLGL--ARPEADQCVAC-A-TLQGLGISW-AI-AFDGDLGMGA-PDAK-ARGDLTLGLEP-PDGK-
IDSECRPRF-ADG-VKVLGSGAF-ADG-QETELVEPL-ADG-RELGSGLAL-A-QEVQGYVLI-ALG-ERGAPPSTF-A-
QEFAGCKKI--MELAALCRW-ALG-VKIPVAIKV-AL-LHCPALVTY;
(SEQ ID NO: 391)
LRIVRGTQL-PIAA-GGGDLTLGL--ARPEADQCVAC-AI-AFDGDLGMGA-PDAK-ARGDLTLGLEP-PDLK-QETELVEPL-PI-
VKVLGSGAF--ASEGAGSDVF-PDG-RELGSGLAL-A-QEVQGYVLI-ADGK-EECRVLQGL-PDLK-LEEITGYLY-A-
TEILKGGVL-PL-EEITGYLYI-AD-MELAALCRW-AD-ARPDLSVFQNL-ADL-TDFGLARLL-PD-TRTVCAGGC;
(SEQ ID NO: 403)
CELHCPALV-ADG-GENVKIPVA--ALPASPETHL-RD-ARPEGRYTFGA-ADGK-IDSECRPRF-ADLK-GERLPQPPI-AIL-
AEEAPRSPLA-ADGA-EEITGYLYI--ALPAARPAGA-PDGK-MEHLREVRA-PDG-RELQLRSLT-ADLK-KEILDEAYV-AT-
AFDGDLGMGA-PDLK-REVRAVTSA--ALPSETDGYV-ADG-AEQRASPLT-ADG-AGEGLACHQL-ADG-RELGSGLAL-AD-
CEKCSKPCA-ADGV-QEVQGYVLI-ADL-TSANIQEFA-AD-LDSTFYRSL--MELAALCRW-ATGK-AINCTHSCVD-RD-
AFEDNYALAV-RD-LGMGAAKGL--VSRLLGICL-PD-VKIPVAIKV-AI-ASCVTACPY;
(SEQ ID NO: 406)
CRWGLLLAL-PD-ENVKIPVAI--AYGVTVWELM-A-ALPASPETHL--ARPDLSVFQNL-PD-LPTNASLSF-ADG-ALPTHDPSPL-
PDL-ALPSETDGYV-PDLK-LGMEHLREV-AD-LPQPPICTI-ADGV-QEVQGYVLI-AD-EQLQVFETL-A-LGMGAAKGL-PD-
KGMSYLEDV-A-QEFAGCKKI-S-VGILLVVVL--AMPNQAQMRI-ADLK-LQLRSLTEI-AD-VKIPVAIKV-A-TDFGLARLL;
(SEQ ID NO: 415)
ASPLDSTFYR-ADG-VENPEYLTP-A-ALPASPETHL--ARAGVGSPYVS-RD-LPTNASLSF-ADG-ALPTHDPSPL-ADL-
LERPKTLSP-AL-AFDGDLGMGA-PDAK-ARGDLTLGLEP-PDL-ARDDMGDLVDA-PDL-ARPEDECVGE-A-TPTAENPEY-AL-
AMPNQAQMRI-ADLK-LPQPPICTI-AD-ASPLTSIISA-AD-CRWGLLLAL--AGPLPAARPA-PD-AAPRSPLAPS-ALA-
ASPQPEYVNQ-ALG-VKIPVAIKV-AD-ACPSGVKPDL-AD-LHCPALVTY-SDA-SPAFDNLYY;
(SEQ ID NO: 425)
AWKDIFHKNN-AD-AFDGDLGMGA-PDLK-REVRAVTSA-ALL-AEEAPRSPLA-ADG-ARDGDPASNTA--ALPAARPAGA-A-
IWIPDGENV-SD-LRENTSPKA-RD-LVEPLTPSG-ADG-LTSIISAVV-A-RKVKVLGSG-ADGV-RELQLRSLT-ADLK-
LPQPPICTI-AD-LQRLRIVRG-PDLK-RGRILHNGA-AD-ASPLTSIISA--ASPLAPSEGA--ACPALVTYNT-AD-
AVPLQRLRIV-ADAA-AMPNQAQMRI-ADLK-AYKDPPFCVA-RDL-AMPIWKFPDE-ADG-AMPYGCLLDH-ADGK-WGLLLALLP;
(SEQ ID NO: 428)
MELAALCRW-A-VTSANIQEF-ALGK-ENVKIPVAI-ADGK-DIFHKNNQL-RD-ATLERPKTL--LVVVLGVVF-P-TLQGLGISW-
A-DVFDGDLGM-RDLV-ALCRWGLLL-PDGK-ISWLGLRSL--RSLLEDDDM-ADG-GSGAFGTVY-ADA-GTQLFEDNY-RDLK-
LSYMPIWKF-ADLK-PAFDNLYYW-ADL-QLMPYGCLL-PDLK-MSYLEDVRL-R-DVWSYGVTV-PDLK-RFTHQSDVW-ADLV-
HTVPWDQLF;
(SEQ ID NO: 436)
PAFDNLYYW-AIL-CTIDVYMIM-ADLV-RMARDPQRF-AD-KGCPAEQRA-PDLK-LGSQDLLNW--AIISAVVGIL-AL-
RCEKCSKPC-AIL-VTSANIQEF-ADL-GAMPNQAQM-AD-AVTGASPGGL-P-ISAVVGILL-PD-RSGGGDLTL--AYLSTDVGSC-
A-LAALCRWGL-AL-ASCVTACPY-ADL-HTVPWDQLF-ADLK-LSYMPIWKF-ADG-RASPLTSII-ADG-VTVWELMTF-ADGV-
ARGQECVEEC-ADL-RIVRGTQLF-TRTVCAGGC-AD-KIFGSLAFL-PD-VCTGTDMKL-AD-LCYQDTILW,
and
(SEQ ID NO: 453)
AKFVAAWTLKAAAKKAVVGILLVVVLGVVFGILIKRRQQKIRKKPICTIDVYMIMVKCWMIDSEKKAQMRILKETELRKVKVLGSGAKK
IKWMALESILRRRFTHQSDVKKPICTIDVYMIMVKCWMIDSRKRSHAGYQTI.

In a preferred embodiment, the polyepitope construct consists of the sequence

(SEQ ID NO: 456-universal)
MELAALCRWGLLLALLPPGAPDGENVKIPVAIKVLRENTADGKEECRVLQ
GLPDGKYSEDPTVPLPDDEAYVMAGVADLKQETELVEPLTPPDGRASPLT
SIISAVVGILLVVVLGVVFPDAGMEHLREVRADGKDIFHKNNQLPDLQPE
QLQVFRDAQEVQGYVLIPDLAFDGDLGMGAPDLQVIRGRILPDVKVLGSG
AFGTVYPIGDLTLGLEPPDLKASCVTACPYATLQGLGISWLGLRSLRELG
SGLALPMQIAKGMSYALFGPEADQCVPDLKLSYMPIWKFADLKPLQRLRI
VRGTQLFEDNYALAVARGAPPSTFKAGVVKDVFAFRDLVKITDFGLARLL
PLVHRDLAARADVWSYGVTVRDTTPVTGASPRDLYISAWPDSLRTVCAGG
CARSDKIFGSLAFLPDLHCPALVTYADDSTFYRSLLADGKQLMPYGCLLA
DGGSCTLVCPLAKFVAAWTLKAAAKKAVVGILLVVVLGVVFGILIKRRQQ
KIRKKPICTIDVYMIMVKCWMIDSEKKAQMRILKETELRKVKVLGSGAKK
IKWMALESILRRRFTHQSDVKKPICTIDVYMIMVKCWMIDSRKRSHAGYQ
TI.

In another preferred embodiment, the polyepitope construct consists of the sequence

(SEQ ID NO: 457-HLA-A*0201-specific)
MELAALCRWGLLLALLPPGAPPDLLALLPPGAPDATLEEITGYLAILDEA
YVMAPILHNGAYSLPQLFEDNYALSIISAVVGIAQLMPYGCLLRLLVVVL
GVVRDLQLRSLTEIAILLVVVLGVPDAVVGILLVVADALCRWGLLLADYI
SAWPDSLRDKIFGSLAFLAKFVAAWTLKAAAKKAVVGILLVVVLGVVFGI
LIKRRQQKIRKKPICTIDVYMIMVKCWMIDSEKKAQMRILKETELRKVKV
LGSGAKKIKWMALESILRRRFTHQSDVKKPICTIDVYMIMVKCWMIDSRK
RSHAGYQTI.

In yet another preferred embodiment, the polyepitope construct consists of the sequence

(SEQ ID NO: 458-HLA-B*3501-specific)
MELAALCRWGLLLALLPPGAPADGKTPTAENPEYAALPASPETHLPILKY
SEDPTVPLPDGALPTHDPSPLADNKEILDEAYADEILDEAYVMPLVVVLG
VVFADMQIAKGMSYALMTFGAKPYPLGKAPPPAFSPAFADLHCPALVTYA
KFVAAWTLKAAAKKAVVGILLVVVLGVVFGILIKRRQQKIRKKPICTIDV
YMIMVKCWMIDSEKKAQMRILKETELRKVKVLGSGAKKIKWMALESILRR
RFTHQSDVKKPICTIDVYMIMVKCWMIDSRKRSHAGYQTI.

In conjunction with the polyepitope constructs of the invention, provided herein are pharmaceutical compositions comprising such polyepitope constructs and a pharmaceutically acceptable carrier or excipient.

Further provided herein are nucleic acids encoding such polyepitope constructs, pharmaceutical compositions comprising such nucleic acids and a pharmaceutically acceptable carrier or excepient, and host cells comprising such nucleic acids.

In another aspect, the invention provides a method for inducing T cell responses in mammals comprising administering to said mammals polyepitope constructs of the invention or nucleic acids encoding such polyepitope constructs.

In yet another aspect, the invention provides a method for treating a HER2-positive breast cancer in mammals comprising administering to said mammals polyepitope constructs of the invention or nucleic acids encoding such polyepitope constructs.

The present invention is further explained below using detailed disclosure and specific examples. Such description, materials, methods, and examples are illustrative only and not intended to be limiting. All cited literature references, patents and patent applications are incorporated herein in their entireties.

### BRIEF DESCRIPTION OF THE DRAWINGS

FIGS. 1A-B show the results of cytotoxicity assays. T-cell immunity was stimulated ex vivo by autologous dendritic cells (DCs) transfected either with pHER2 (positive control), or with plasmids coding for polyepitope constructs of the invention (pBCU—“universal” one—containing HER2 epitopes, predicted to be the most promiscuous MHC-binders, car pBCA0201 —containing HER2 epitopes restricted by HLA-A*0201), or with plasmid prHA5 coding for an unrelated protein rHA5 corresponding to a portion (aa 17-346) of Influenza A virus H5N1 hemagglutinin (HA). Unstimulated non-adherent mononuclear cells (None) were used as negative controls, Either autologous DCs transfected with pHER2 (A) or MCF-7 breast cancer cells (HER2+/HLA-A*0201+) (B) were used as target cells. Cytotoxicity was assessed at different ratio of effector to target cells (10:1, 20:1, 30:1). Statistical significance of observed differences between the groups was assessed using Wilcoxon rank-sum test. P<0.05 was considered to be significant.

FIGS. 2A-B show the levels of γIFN production by T-cells determined by intracellular cytokine staining followed by flow cytometry. Results are represented as percent (%) of double-positive T-cells as compared to the total number of either CD8+ (A) or CD4+ (B) (1×10⁵ cells). None—unstimulated non-adherent mononuclear cells (MNCs) (negative control); DC:prHA5—MNCs stimulated by DCs transfected with prHA5 (negative control); DC:pHER2—MNCs stimulated by DCs transfected with pHER2; DC:pBCU—MNCs stimulated by DCs transfected with pBCU; DC:pBCA0201—MNCs stimulated b DCs transfected with pBCA0201. MCF-7 cancer cells were used as target cells in these experiments. Statistical significance of observed differences between the groups was assessed using Wilcoxon rank-sum test. P<0.05 was considered to be significant.

### DETAILED DESCRIPTION OF THE INVENTION

The present invention is based on development of new methods for arranging immunogenic epitopes into polyepitope constructs aimed at optimizing proteasome and/or immunoproteasome processing of the polyepitope and optimizing TAP-binding of released epitopes. The new methods of the invention are based on the novel algorithm of epitope arrangement which allows to choose appropriate epitope matchings and spacer sequences taking into account predicted efficiency of proteasonial processing, spacer length and the number of predicted “non-target” CTL-epitopes resulting from artificial junction of epitopes through the spacer. These new methods of the invention lead to generation of novel HER2-specific polyepitope constructs (also disclosed herein) which are characterized by greatly enhanced antigen presentation as compared to the native HER2 antigen.

The present invention provides immunogenic polyepitope constructs comprising two or more different T cell epitopes, which epitopes are CTL epitopes or T-helper (Th) epitopes and are derived from one or more disease-associated antigens or pathogens, and wherein the epitopes are optionally joined by spacer sequences which improve the immunogenicity of the polyepitope construct by providing efficient proteasome and/or immunoproteasome processing of the epitopes and enhancing their interaction with Transporters Associated with Antigen Processing (TAP). As compared to the use of whole protein antigens, the use of the spacer-containing polyepitope constructs of the invention results in an enhanced efficiency of epitope presentation by antigen presenting cells (APCs).

The polyepitope constructs of the invention can comprise CTL epitopes or Th epitopes or both. CTL and Th epitopes can be either mixed within a construct or can be arranged into separate CTL and Th epitope clusters. In a separate embodiment, the invention provides a combination of two or more polyepitope constructs, wherein at least one of the constructs is CTL epitope-only or Th epitope-only. Th epitopes are primarily useful to stimulate CD4+ responses, and CTL epitopes are primarily useful to stimulate CD8+ T-cell responses. The present invention also encompasses combinations of two or more different polyepitope constructs. To induce an effective T-cell immune response, it is important to induce both CTL (CD8+) and Th (CD4+). Thus, the preferred polyepitope constructs of the present invention include both CTL and Th epitopes.

The sequences of the different epitopes within polyepitope constructs of the invention can be derived from any part of a polypeptide antigen and can overlap to some degree (i.e., share from at least one amino acid residue to all but one amino acid residue) or they can be non-overlapping. The epitopes used within the construct can be arranged in any order as compared to the antigen from which they are derived. Epitopes used within polyepitope constructs of the invention can be of any specified length but are preferably at least 8 amino acids in length. CTL epitopes are preferably 8-12 amino acids in length. Th epitopes are preferably 9-25 amino acids in length. The MHC class alleles to which the epitopes in the polyepitope constructs of the present invention bind can be any human class I or II allomorphs, e.g., HLA-A*0101, HLA-A*0201, HLA-A*0301 etc. A given epitope may be promiscuous, i.e., bind more than one MHC allotype. Preferably, the epitopes used in the polyepitope constructs of the invention are promiscuous MHC-binders. A representative list of class I-binding epitopes of the HER2 protein, any of which can be included in the polyepitope constructs of the invention, is provided in Example 2.1.1, below. A representative list of class II-binding epitopes of the HER2 protein any of which could be included in the polyepitope constructs of the invention, is provided in Example 2.3.1.1, below. Examples of epitopes selected for 30 human MHC class I alleles are provided in Example 2.2.1, below. These epitopes can be used either to construct “universal” polyepitope constructs aimed to evoke cellular immune responses in the majority of humans, or to produce “allele-specific” polyepitope constructs specific for certain HLA alleles.

The polyepitope constructs of the invention can be specific for a particular disease-associated antigen or pathogen (including two or more strains of the same pathogen), or can contain epitopes derived from two or more different antigens or pathogens. In one preferred embodiment, the polyepitope constructs of the invention comprise epitopes of HER2 protein.

The use of individual epitopes within the constructs of the invention allows to achieve efficient MHC class I and MHC class II-dependent antigen presentation even when only a partial sequence of a disease-associated antigen or pathogen is available (e.g., in cases of newly discovered pathogens or tumor antigens). The use of individual epitopes as opposed to whole antigens also allows to avoid problems associated with interference with antigen presentation by certain protein antigens (e.g., viral or bacterial proteins down-regulating host immune responses, down-regulating expression of MHC molecules on the cellular surface, interfering with cytokine signaling etc.), or deleterious effects (e.g., toxicity) associated with over-expression of particular viral proteins or tumor antigens.

An important additional advantage of the present invention is that the assortment of epitopes within the polyepitope constructs increases the likelihood that at least one epitope will be presented by each of a variety of HLA allotypes. This allows for immunization of a population of individuals polymorphic at the HLA locus, using a single polyepitope construct or a nucleic acid encoding such polyepitope construct. Alternatively, the polyepitope construct can be specific for a particular HLA allotype (e.g., if can contain epitopes with certain HLA-specificity).

In a specific embodiment, the polyepitope constructs of the invention further comprise Th epitopes which are not derived from a disease-associated antigen or pathogen but enhance the CD4+ T-cell responses to the antigen or pathogen (e.g., Pan DR T Helper Epitope [PADRE epitope] AKFVAAWTLKAAA [SEQ ID NO: 1]).

The use of the spacer sequences in the polyepitope constructs of the invention is optional, and two or more of the epitopes can be contiguous (i.e., joined end-to-end) with no spacer between them.

The spacer sequences used in the polyepitope constructs of the invention are degenerate spacer motifs which are optimized for every pair of epitopes to provide the best processing efficiency using novel algorithms of epitope arrangement and sequence optimization. The spacer sequences useful in the polyepitope constructs of the invention can consist of a single amino acid residue or a sequence of two or more amino acids inserted between two neighboring epitopes (or between an epitope and other sequences) of the construct. Preferably, such spacer sequences consist of up to 6 amino acids. However, spacer sequences of up to 7, 8, 10, 15, 20, 30, or 50 amino acids and even longer sequences are also possible. Spacer sequences are useful for promoting proteolytic processing of polyepitope constructs to release individual epitopes for antigen presentation. The spacers sequences are typically removed from the epitope sequences by proteolytic processing within antigen-presenting cell (APC). This leaves the epitopes intact for binding to MHC molecules. Occasionally, a spacer amino acid or part of a spacer sequence will remain attached to an epitope through incomplete processing. This generally will have little or no effect on binding to the MHC molecule. In one preferred embodiment, the spacer used to connect two or more Th epitopes within the polyepitope construct has the core sequence K/R-K/R, which corresponds to cleavage sites recognized by cathepsins B and L.

In another preferred embodiment, the spacer connecting two CTL epitopes can be derived from the following amino acids in the corresponding positions: [AGKNPRS][ADGILTV][AEGKLNV][AFIKLNSV][AEGIKLPSV][AEGKLSV] (SEQ ID NO: 463). This degenerate motif can be used as a basis for selection of spacer sequences for optimizing processing. While preferred length of spacer sequences is about 3-4 amino acids, the invention encompasses both shorter and longer sequences. E.g. two epitopes would be joined without any spacer (using blank spacer) if they could be joined end-to-end according to the scoring function.

In a specific embodiment, polyepitope constructs of the invention further comprise N-terminally conjugated modified ubiquitin (e.g., ubiquitin with G76V substitution [UbV76]), which further enhances proteasomal processing of the epitopes contained in the construct and also enhances CTL-responses. UbV76 can be fused directly to the amino terminus of the polyepitope construct or Arg or Val residue can be inserted between UbV76 and polyepitope construct to stabilize the resulting chimeric constructs (Andersson H. A., Barry M. A., 2004, Mol Ther, 10(3):432-446).

In a specific embodiment, the polyepitope constructs of the invention further comprise one or more targeting signals which direct intracellular transport of the construct to the specific compartment of the cell. Non-limiting examples of useful targeting signals include, for example, (i) homologous or heterologous signal peptides targeting constructs to the secretory pathway via the endoplasmic reticulum (ER) and trans-Golgi network (e.g., the signal peptide of HER2 protein) and (ii) endosome-targeting signals (e.g., a portion or the whole sequence of the invariant chain associated with MHC class II molecules; C-terminal portion of the human LAMP-1 protein, the tyrosine-motif Y-X-X-hydrophobic amino acid, wherein X is any amino acid). A preferred targeting signal useful in the polyepitope constructs of the invention includes both C-terminal portion of LAMP-1 and the signal peptide of HER2 protein. This targeting signal is useful for upregulating MHC class II-dependent antigen presentation and CTL response (because the signal peptide of HER2 protein contains CTL epitopes). The targeting signals used in the constructs of the present invention can be optionally modified to introduce an amino acid substitution or spacer sequences at the junction(s) between the targeting signal and the adjacent segment(s) to promote cleavage of the targeting sequence(s) from the epitopes by, e.g., a signal peptidase. The targeting sequences useful in the polyepitope constructs of the invention can contain substitutions of any amino acid except those relevant for targeting.

In conjunction with the polyepitope polypeptide constructs of the invention, provided herein are nucleic acids encoding such polyepitope polypeptide constructs, vectors comprising such nucleic acids (e.g., plasmid, bacterial, and viral vectors), and host cells which comprise such nucleic acids or vectors (e.g., dendritic cells (DC), Langerhans cells, or other antigen presenting cells). When the polyepitope constructs of the invention are administered as nucleic acids and/or using various delivery vehicles (e.g. microparticles, virus-like particles, etc.), such nucleic acids and/or delivery vehicles can further enhance the antigen-specific immune responses (e.g., by promoting IL-12 and γ-interferon (γIFN) release from macrophages, NK cells, and T cells).

The present invention further provides pharmaceutical compositions comprising (i) the polyepitope polypeptide constructs of the invention or nucleic acids encoding such polyepitope polypeptide constructs or vectors comprising such nucleic acids and (ii) a pharmaceutically acceptable carrier or excipient. Such compositions can further comprise a delivery vehicle (such as, e.g., a microparticle).

The polypeptide and nucleic acid constructs and compositions of the invention can be administered via different routes. For example, they can be administered to mucosal tissue (e.g., vaginal, nasal, lower respiratory, or gastrointestinal tissue [e.g., rectal]). Alternatively, they can be administered systemically, for example, intravenously, intramuscularly, intradermally, orally, or subcutaneously.

### 1.1 Definitions

As used herein, the term “tumor antigen” refers to a protein which is expressed exclusively in tumor cells, or is highly upregulated in tumor cells as compared to non-tumor homologs of the tumor cells. Such tumor antigens frequently serve as markers for differentiating tumor cells from their normal counterparts.

The term “epitope” as used herein refers to a T-cell epitope, e.g. an oligopeptide able to bind to either MHC class I or class II molecules and to stimulate T-cell immune responses of appropriate T-lymphocytes. The terms “universal epitope” and “universal polyepitope construct” are used herein to refer to epitopes and polyepitope constructs which evoke cellular immune responses in the majority of immunized population (e.g., humans). The terms “allele-specific epitope” and “allele-specific polyepitope construct” refer to epitopes and polyepitope constructs which evoke cellular immune responses in immunized subjects (e.g., humans) having certain MHC haplotype(s) (e.g., certain HLA alleles).

As used herein, the term “polyepitope” or “polyepitope construct” refers to an immunogenic construct including two or more different epitopes. Such different epitopes may have completely unrelated or related sequences and may overlap in their sequences to some degree (e.g., share at least one amino acid residue or share up to all but one residue), or they may be non-overlapping. A given epitope within the polyepitope need not be of any specified length but is preferably between 8 and 12 amino acids in length for MHC class I-restricted epitopes and preferably between 8 and 25 amino acids in length for WIC class II-restricted epitopes. In the polyepitope constructs of the present invention, two or more adjacent epitopes can be joined end-to-end, with no spacer between them. Alternatively, any two adjacent epitopes can be linked by a spacer sequence, as defined below. The epitopes within the polyepitope constructs of the present invention can be arranged in any order (e.g., such order does not have to reflect the order of these epitopes within the protein they are derived from). The polyepitope constructs of the invention can contain any number of epitopes, but preferably contain at least 5 epitopes (in case of allele-specific constructs) or at least 20 epitopes (in case of universal constructs).

The term “polyCTL” refers to a polyepitope construct including either known or predicted epitopes for CD8+ T-lymphocytes.

The terms “polyThelper” or “polyTh” refer to a polyepitope construct including either known or predicted epitopes for CD4+ T-lymphocytes.

The term “junk epitope” refers to an epitope, not found in original antigen(s) of interest, generated due to artificial conjunction of chosen epitopes and/or spacer sequences within the polyepitope construct.

The term “targeting signal” refers to a sequence which directs intracellular transport of the polyepitope construct to a specific compartment of an antigen-presenting cell (APC).

The terms “spacer sequence”, “spacer” and “flanking sequence” are used interchangeably to refer to a single amino acid residue or a sequence of two or more amino acids inserted between two neighboring epitopes or an epitope and another sequence within a polyepitope construct which improve the immunogenicity of the polyepitope construct by providing efficient proteasome and/or immunoproteasome processing of the epitopes and enhancing their interaction with Transporters Associated with Antigen Processing (TAP).

The term “therapeutically effective” applied to dose or amount refers to that quantity of a polyepitope construct or pharmaceutical composition or vaccine that is sufficient to result in a desired activity upon administration to a mammal in need thereof. As used herein with respect to polyepitope construct-containing compositions or vaccines, the term “therapeutically effective amount/dose” is used interchangeably with the term “immunogenically effective amount/dose” and refers to the amount/dose of a polyepitope construct or pharmaceutical composition or vaccine that is sufficient to produce an effective immune response upon administration to a mammal. According to the present invention, a preferred immunogenically effective amount of the polyepitope construct is in the range of 1-950 μg per kg of the body weight.

The phrase “pharmaceutically acceptable”, as used in connection with compositions of the invention, refers to molecular entities and other ingredients of such compositions that are physiologically tolerable and do not typically produce unwanted reactions when administered to a human. Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans.

The term “carrier” applied to pharmaceutical or vaccine compositions of the invention refers to a diluent, excipient, or vehicle with which a compound (e.g., an antigen and/or an MHC molecule) is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution, saline solutions, and aqueous dextrose and glycerol solutions are preferably employed as carriers, particularly for injectable solutions. Suitable pharmaceutical carriers are described in “Remington's Pharmaceutical Sciences” by E. W. Martin, 18th Edition.

The term “about” or “approximately” usually means within 20%, more preferably within 10%, and most preferably kill within 5% of a given value or range. Alternatively, especially in biological systems (e.g., when measuring an immune responses, the term “about” means within about a log (i.e., an order of magnitude) preferably within a factor of two of a given value.

In accordance with the present invention, conventional molecular biology, microbiology, and recombinant DNA techniques may be employed within the skill of the art. Such techniques are well-known and are explained fully in the literature. See, e.g., Sambrook, Fritsch and Maniatis, Molecular Cloning: A Laboratory Manual, Second Edition (1989) Cold Spring Harbor Laboratory Press, Cold Spring Harbor, N.Y. (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization [B. D. Hames & S. Higgins eds. (1985)]; Transcription And Translation [B. D. Hames & S. J. Higgins, eds. (1984)]; Animal Cell Culture [R. I. Freshney, ed. (1986)]; Immobilized Cells And Enzymes [IRL Press, (1986)]; B. Perbal, A Practical Guide To Molecular Cloning (1984); F. M. Ausubel et al. (eds.), Current Protocols in Molecular Biology, John Wiley & Sons, Inc. (1994).

All other terms found here are used in their common meaning in the specified fields of interest; molecular biology, immunology, cytology, bioinformatics.

### 1.2 Antigens Used as a Source of Epitopes of the Invention

While the specific polyepitope constructs disclosed herein are based on HER2-specific epitopes and are useful for inducing immune response to HER2-expressing breast cancer cells, the same principals as described herein are applicable to all other disease-specific polyepitope constructs. The antigens useful as a source of epitopes in the polyepitope constructs of the present invention include without limitation various viral, bacterial, fungal, parasite-specific, and tumor-specific antigens. Non-limiting examples of viral antigens of the invention include antigens derived from influenza virus (e.g., surface glycoproteins hemagglutinin (HA) and neuraminidase (NA)); immunodeficiency virus (e.g., a human immunodeficiency virus antigens (HIV) such as gp120, gp160, p18 antigen Gag p17/p24, Tat, Pol, Nef, and Env); herpesvirus (e.g., a glycoprotein from herpes simplex virus (HSV), Marek's Disease Virus, cytomegalovirus (CMV), or Epstein-Barr virus); hepatitis virus (e.g., Hepatitis B surface antigen (HBsAg)); papilloma virus; roes associated virus (e.g., RAV-1 env); infectious bronchitis virus (e.g., matrix and/or preplomer); flavivirus (e.g., a Japanese encephalitis virus (JEV) antigen, a Yellow Fever antigen, or a Dengue virus antigen); Morbillivirus (e.g., a canine distemper virus antigen, a measles antigen, or rinderpest antigen such as HA or F), rabies (e.g., rabies glycoprotein G); parvovirus (e.g., a canine parvovirus antigen); poxvirus (e.g., an ectromelia antigen, a canary poxvirus antigen, or a fowl poxvirus antigen); chicken pox virus (varicella zoster antigen); infectious bursal disease virus (e.g., VP2, VP3, or VP4), Hantaan virus, and mumps virus. Non-limiting examples of bacterial antigens of the invention include lipopolysaccharides isolated from gram-negative bacterial cell walls and staphylococcus-specific, streptococcus-specific, pneumococcus-specific (e.g., PspA; sec PCT Publication No. WO 92/14488), Neisseria gonorrhea-specific, Borrelia-specific (e.g., OspA, OspB, OspC antigens of Borrelia associated with Lyme disease such as Borrelia burgdorferi, Borrelia afzeili, and Borrelia garinii [see, e.g., U.S. Pat. No. 5,523,089; PCT Publication Nos. WO 90/04411, WO 91/09870, WO 93/04175, WO 96/06165, WO93/08306; PCT/US92/08697; Bergstrom et al., Mol. Microbiol. 1999; 3: 479486; Johnson et al., Infect. and Immun. 1992; 60: 1845-1853; Johnson et al., Vaccine 1995; 13: 1086-1094; The Sixth International. Conference on Lyme Borreliosis: Progress on the Development of Lyme Disease Vaccine, Vaccine 1995; 13; 133-135]), and pseudomonas-specific proteins or peptides. Non-limiting example of malaria-specific antigen is malarial circumsporozoite (CS) protein. Non-limiting examples of fungal antigens include those isolated from candida (e.g., MP65 from Candida albicans), trichophyton, and ptyrosporum. Non-limiting examples of tumor-specific antigens include WT-1 antigen (in lymphoma and other solid tumors), ErbB receptors, Melan A [MART1], gp 100, tyrosinase, TRP-1/gp 75, and TRP-2 (in melanoma): MAGE-1 and MAGE-3 (in bladder, head and neck, and non-small cell carcinoma); HPV EG and E7 proteins (in cervical cancer); Mucin [MUC-1] (in breast, pancreas, colon, and prostate cancers); prostate-specific antigen [PSA] (in prostate cancer); carcinoembryonic antigen [CEA] (in colon, breast, and gastrointestinal cancers) and such shared tumor-specific antigens as MAGE-2, MAGE-4, MAGE-6, MAGE-10, MAGE-12, BAGE-1, CAGE-1,2,8, CAGE-3 TO 7, LADE-1, NY-ESO-1/LAGE-2, NA-88, GnTV, and TRP2-INT2. Non-limiting examples of autoimmune disease-specific antigens include GAD 65, 1A-2 and insulin B chain (for type 1-diabetes), and myelin basic protein and glatiramer acetate (GA) (for multiple sclerosis).

### 1.3 Algorithm of Epitope Selection

The epitopes useful in the polyepitope constructs of the present invention can be determined using computational methods.

Useful computational methods include, for example, the original TEpredict software (Antonets D. V., Maksyutov A. Z 2010, MolBiol 44(1):130-139; http://tepredict.sourceforge.net). Predictive models for TEpredict were built using partial least squares (PLS) regression on the basis of known peptide-HLA binding data, taken from IEDB (immune Epitope Database, http://www.epimmune.org). Models, included in TEpredict, use scales of physicochemical properties of aminoacids to parametrize peptides.

Predictive models useful for the present invention can be represented with the following general formula:

$${pIC}_{50} = {\sum }_{i = 1}^{9} {ω}_{i} {P}_{i} + const ,$$

where pIC₅₀ is the measure of MHC-peptide binding affinity, Pᵢ is a vector of properties, encoding amino acid a at position i in the peptide; ωᵢ is a vector with weights of these properties.

There are numerous other algorithms which can be used for defining T-cell epitopes useful in the polyepitope constructs of the present invention. One non-limiting example is artificial neural network-based methods developed by Lundegaard et al. (Lundegaard C. et al. 2008. NAR, 36:W509-512).

In one embodiment of the present invention, predictions of MHC class I-binding epitopes were made for 30 different HLA alleles (HLA-A*0101, A*0201, A*0202, A*0203, A*0206, A*0301, A*2301, A*2402, A*2403, A*2601, A*2902, A*3001, A*3002, A*3101, B*0702, B*0801, B*1501, B*1801, B*2705, B*3501, B*4001, B*4002, B*4402, B*4403, B*4501, B*5101, B*5301, B*5401, B*5701, B*5801). The predicted value of pIC50 greater then 6.8 was chosen to differentiate binders from non-binders.

Making prediction of peptide-TAP binding affinity before the prediction of MHC class I-binding epitopes was shown to lower the rate of false positive prediction results (Peters B et al., 2003, J. Immunol, 171:1741-1749), thus the prediction of peptide-to-TAP binding can be used for selection of potential T-cell epitopes in the methods of the present invention. TAP-binding prediction can be used as a filter to avoid selecting epitopes which inefficiently interact with TAP or as a ranking function to weight peptides according to their predicted TAP-binding affinity. Prediction of peptide-TAP binding can be done using algorithms implemented in TEpredict or using other relevant computational tools. E.g., in one of the specific embodiments of the present invention, from 1247 peptides of the HER2 protein 860 peptides were selected using TAP-binding affinity prediction. TAP binding prediction implemented in TEpredict is based on predictive model and algorithms developed by Peters et al. (J. Immunol, 2003, 171:1741-1749).

Prediction of proteasome and/or immunoproteasome, cleavage of protein antigen of interest can be applied to choose peptides possessing a cleavage site at their C-terminus (proteasome was shown to generate C-terminus of naturally occurring MHC I-binding epitopes). Prediction of proteasome and/or immunoproteasome processing can also be used either as a filter or as a ranking function. In one embodiment of the present invention, 338 peptides from HER2 protein were selected using a combination of proteasome and immunoproteasome filters. Algorithms for predicting proteasomal and/or immunoproteasomal processing of protein antigens which were implemented in TEpredict software were based on predictive models developed by Toes et al. (Toes R E et al., 2001, J. Exp. Med, 194:1-12). Determination of threshold levels for predicting proteasome processing is described in. e.g. Singh and Raghava (Singh H and Raghava G P, 2003, Bioinformatics, 19:1009-1014).

While such additional steps of selection can lead to false negative results, they can be advantageous in terms of immunodominance. E.g., peptides, selected using these filters and predicted to bind to TAP and to have proteasomal cleavage site on their C-terminus, are likely to be more efficiently released in vivo. Indeed, Peters et al. (J. Immunol, 2003, 171:1741-1749) and Doytchinova et al. (J. Immunol, 2004, 173:6813-6819) had shown that preselection of peptides predicted to efficiently bind to TAP lowered the number of false-positive results when predicting T-cell epitopes.

Specific non-limiting examples of predicted epitopes chosen for inclusion into polyepitope constructs of the present invention are provided in Examples, below.

In one embodiment of the present invention, promiscuous MEW class I- or class II-binders were selected using greedy algorithm. This algorithm allows to choose the minimal number of peptides to cover the diversity of selected MHC allotypes. The epitopes were selected with five-fold redundancy, i.e., at most five potential epitopes for every MHC allotype, used for predictions, were contained in the created set. This was thought to be important due to extremely high polymorphism of HLA genes. This algorithm was created to cover the majority of individuals in the populations of interest by the smallest number of peptides; to create a redundant set of promiscuous epitopes to construct a “universal” set of peptides able to evoke immune responses in the majority of humans, Non-limiting examples of selected MHC class I and class II-binders are provided in Examples, below.

In an alternative set of embodiments, HLA allele-specific polyepitope constructs were created for vaccination of individuals with specified HLA alleles. In one such embodiment, HLA allele-specific sets were created for 30 different HLA class I alleles. Two different sets were created for each allele using two different prediction algorithms. These sets are listed in Table 3, below.

### 1.4 Algorithms for Combining Epitopes into Polyepitope Constructs of the Invention

1.4.1 Methods for optimizing Epitope interaction with TAP

To make processing of epitopes within polyepitope constructs of the invention more efficient the present inventors have developed novel spacer (flanking) sequences aimed to optimize peptide binding to TAP. In the specific computational methods disclosed herein, TAP-binding affinity was predicted for every epitope within the polyepitope construct and spacer sequences were added only to peptides predicted to be inefficient TAP-binders.

In one specific embodiment of the present invention, an algorithm for choosing spacer sequences to optimize TAP binding is based on matrices and methods developed by Peters et al. (J. Immunol. 2003, 171:1741-1749) included in TEpredict. In this algorithm, affinity of peptide-TAP binding is calculated according the formula: N1+N2+N3+C, where N1 corresponds to contribution of the first N-terminal amino acid, N2—of the second amino acid from the N-terminus of the peptide, N3—of the third amino acid from the N-terminus of the peptide, and C is the contribution of the last (C-terminal) amino acid. In this algorithm, C-terminus needs to be unchanged (because it was shown that there are no active carboxypeptidases within endoplasmic reticulum (ER), and thus proteasomal processing is believed to provide C-terminus of the epitope while the N-terminus of the peptide could be trimmed by ERAPs (ER aminopeptidases)) and only N-terminal amino acids can be added to improve TAP binding. In one specific embodiment, ARY motif and its shorter derivatives were chosen as the N-terminal spacer sequence. First, Ala (A) residue was added to the epitope and if that peptide was predicted to be inefficient TAP hinder, Ala-Arg (AR) motif was added to the epitope. If that peptide was predicted to bind to TAP with low affinity then Ala-Arg-Tyr (ARY) motif was added to the epitope. For many of the epitopes used in the polyepitope constructs of the present invention, only a single Ala residue was needed for efficient interaction with TAP. In another embodiment, a degenerate motif for optimization of peptide binding to TAP was used, e.g. [ANRK][RQYM][YWFVI] (SEQ ID NO: 464).

1.4.2 Methods for Optimizing Proteasome and/or Immunoproteasome Processing of Epitopes

In the methods of the present invention, to optimize proteasome and/or immunoproteasome release of epitopes from the polyepitope constructs of the invention, spacer sequences need to be determined for every pair of epitopes. This can be done using, for example, the two different algorithms described below.

The first algorithm is based on the use of 6 amino acid—long consensus spacer sequence ADLVKV (SEQ ID NO: 2), which is optimal for both proteasome and immunoproteasome processing. For optimization of the release of C-termini of epitopes, ProPred1 matrices can be used (Toes R E et al., 2001, J. Exp, Med, 194:1-12; Singh H., Raghava G. P., 2003, Bioinformatics, 19(8):1009-14). For combination analysis and data presentation, directed graphs can be used, where peptides are nodes of the graph and edges connecting nodes A and B define the combinations, where the necessary cleavage site is present at the C-terminus of peptide A.

Other spacer sequences can be used with the same algorithm. For example, sequence ADLVAG (SEQ ID NO: 3) can be used to optimize proteasome processing, and sequence ADLAVK (SEQ ID NO: 4) can be used to optimize immunoproteasome processing. Degenerate variants of these spacer sequences can be also used, wherein any amino acid from the sequence can be replaced by any of the 20 naturally occurring amino acids. All amino acids within the spacer can be replaced simultaneously. Furthermore, the spacer can be shorter or longer than 6 amino acids in length. However, the spacer selection is not random, since the selection of spacer sequence for every pair of epitopes is made according to the scoring function. When a spacer sequence between epitopes A and B is predicted, the preference is given to amino acids providing the most efficient release of the C-terminus of epitope A. Determination can be performed using models incorporated within TEpredict or any other model for predicting proteasome and/or immunoproteasome processing.

This version of algorithm for constructing a polyepitope construct of the invention can be presented by the following sequence of steps:

1. addition of spacer sequences (for optimization of epitope interaction with TAP) for all chosen epitopes (if needed);

2. testing of spacer sequences from the group consisting of ‘ ’, ‘A’, ‘AD’, ‘ADL’, ‘ADLV’ (SEQ ID NO: 5), ‘ADLVK’ (SEQ ID NO: 6), ‘ADLVKV’ (SEQ ID NO: 2), until the resulting construct contains all requisite chosen epitopes or until all spacer sequences are tested.

If the resulting construct does not include all requisite chosen epitopes:

2.1. a graph is constructed;

2.2. if the graph contains adjacent vertices, choose the path with the maximal length;

2.3. exclude vertices corresponding to peptides included in the chosen path;

2.4. add to the selection peptide(s) corresponding to the chosen path;

2.5. see point 2.2;

2.6. if the graph does not contain adjacent vertices, create a new selection of peptides consisting of chosen paths and remaining nodes of the graph; go back to the new cycle (point 2).

3. as a result, a sequence of the polyepitope construct should be obtained; if the path was not chosen, which included all epitopes, repeat algorithm from point 2 at a lower stringency of proteasome/immunoproteasome filter.

The present invention also encompasses various modifications of the above algorithm. For example, an additional cycle can be included which uses different values of stringency of proteasome/immunoproteasome filter.

The second approach is based on the use of a degenerate optimal spacer sequence [APRS][DILT][AGL][AKV] (SEQ ID NO: 460) for optimizing proteasome and/or immunoproteasome processing. This sequence is used to create a selection of spacer sequences of 1-4 amino acids in length, which selection includes more than 150 different sequences. Other degenerate optimal spacer sequences can be also used. For example, [ARSPNK][DLITGV][LGAVEK][VKAFSI][ALKSEI][GVKLSE] (SEQ ID NO: 461) can be used as a basis for selection of spacer sequences for optimizing proteasome processing, and [AGNRKP][DIATVG][LGANVE][ASNVLK][VIKAGP][KAGVSE] (SRO ID NO: 462) can be used as a basis for selection of spacer sequences for optimizing immunoproteasome processing. While preferred length of spacer sequences is about 3-4 amino acids, the invention encompasses both shorter and longer sequences. Degenerate variants of the spacer sequences can be also used with amino acid changes in positions which do not affect proteasome and/or immunoproteasome processing.

When the above second approach is used for each combination of epitopes A and B, the selected spacer sequence is the sequence which allows for efficient proteasome cleavage at the C-terminus of epitope A, predicted at a given level of stringency of the proteasome filter. Briefly, the filter works as follows: for any overlapping nanomeric peptides extracted from the antigen sequence the probability of proteasonial cleavage site on its C-terminus is predicted; if predicted score is less than selected threshold value then the peptide, is excluded from further analysis. See also Toes R E et al., 2001, J. Exp. Med, 194:1-12; Singh H., Raghava G. P., 2003, Bioinformatics, 19(8):1009-14. For all selected variants, epitope prediction is conducted, and one prediction is chosen for each pair of peptides (using criteria described below). Then a polyepitope construct is assembled, wherein the first peptide is used as a function argument, or is selected automatically (as the best based on chosen criteria). If any given peptide is not included in the final polyepitope construct, the algorithm searches for peptides, which can be used for insertion of this omitted peptide. If no place for insertion is found, the omitted peptide is used as a starting peptide.

The following criteria can be used for choosing, the spacer sequence for peptides A and B: the number of junk epitopes predicted for a given spacer; the number of MHC allomorphs, which interact with these junk epitopes; the length of the spacer (normally, the shorter spacers are preferred), All variants of spacer sequences are arranged by predicted efficiency of the release of the C-terminus of peptide A. These criteria can be used as filters; they can be used together or separately, and in different sequence. Also, the stringency of prediction of potential T-cell epitopes and proteasome and/or immunoproteasome processing of peptide fragments can be varied.

The above criteria are used for selecting the first pair of peptides (if the first peptide was not previously defined) and for selecting each following peptide,

1.4.3 Methods for Minimizing the Number of “Junk” Epitopes

While literature describes induction of T-cell immune responses to all antigenic peptides which can be presented by allelic variants of MHC molecules of a given organism, the present inventors believe that it is important to minimize the number of “junk” epitopes which are formed at the junctions of epitopes within the polyepitope constructs of the present invention. Minimizing the number of junk epitopes is important, because such epitopes can gain immunologic advantage by being heterologous for a given organism, and T lymphocytes which can interact with them have not been subjected to negative selection. The second algorithm for constructing the polyepitope constructs of the present invention provided above was created in part for solving this problem. See also, Example 2.1.2, below.

### 1.5 The Algorithm for Selection and Joining of Th Epitopes

The above methods address selection and arrangement of CTL epitopes which are used for induction of CD8+ T-lymphocytes. Preferably, the polyepitope constructs of the present invention also contain Th epitopes which are used for induction of CD4+ T-lymphocutes.

Th epitopes can be predicted using, for example, TEpredict. Also, a universal immunogenic peptide PADRE (Pan DR T Helper Epitope) can be used, since it interacts with a large number of common HLA-DR allomorphs as well as murine I-A^{b}.

The following fragments containing Th epitopes for most ErbB2 MHC II allomorphs were chosen for predictions:

(SEQ ID NOS: 7, 8, 9, 10, 11 respectively)
AVVGILLVVVLGVVFGILIKRRQQKIR,
PICTIDVYMIMVKCWMIDSE,
AQMRILKETELRKVKVEGSGA,
IKWMALESILRRRFTHQSDV,
PICTIDVYMIMVKCWMIDS

When these fragments were chosen, 3-5 amino acids flanking the epitope were included as potentially important for interaction with certain T-cell receptors.

The peptides were joined by KK motifs which correspond to sites for cleavage by lysosomal catepsins B and L.

(SEQ ID NO: 12) [symbol] [symbol]
KKAVVGILLVVVLGVVFGILIKRRQQKIRKKPICT
IDVYMIMVKCWMIDSEKKAQMRILKETELRKVKVLGSGAKKIKWMALESI
LRRRFTHQSDVKKPICTIDVYMIMVKCWMIDS (PADRE is in
bold; spacer sequence are underlined)

### 1.6 Targeting Signals Useful in the Polyepitope Constructs of the Invention

Numerous studies have demonstrated that inclusion of N-terminal signal sequences of various proteins and C-terminal lysosomal sorting sequence from human LAMP-1 protein in immunogenic constructs results in high level of Th response as compared to constructs which do not contain such targeting signals (Bonini C. et al. Greenberg P. D. Jour Immunol, 2001, 166(8):5250-5257; Su Z. et al. 2002, 62(17):5041-5048; Bonehill A. et al. Jour Immunol, 2004, 172(11):6649-57; Fassnacht M. et al. Clinical Cancer Res, 2005, 11(15):5566-71). The use of N-terminal signal sequences ensures targeting to ER and secretory pathway, while the use of the C-terminal lysosomal sorting sequence from human LAMP-1 protein ensures targeting of the associated immunogen from the secretory pathway into lysosomes for degradation, where peptide fragments bind to MHC-II molecules leading to their presentation on the cell surface.

A preferred IN-terminal targeting signal used in the polyepitope constructs of the present invention is a slightly modified version of the HER2 signal peptide: MELAALCRWGLLLALLPPGAP (SEQ ID NO: 13) or the original HER2 signal peptide MELAALCRWGLLLALLPPGAAS (SEQ ID NO: 14).

Carboxy terminal sorting signal can be the last 11 amino acids of the LAN/IP-1 protein: RKRSHAGYQTI (SEQ ID NO: 15). A longer fragment of LAMP-1 can be also used as a sorting signal, e.g. the last 34 amino acids: IPIAVGGALAGLVLIVLIAYINGRKRSHAGYQTI (SEQ ID NO: 16)—transmembrane and cytoplasmic domains.

Two non-limiting examples of preferred polyepitope constructs of the present invention are as follows:
1. N-signal|PolyTh|PolyCTL|LAMP-1
2. N-signal|PolyCTL|PolyTh|LAMP-1

As specified above, combinations of all-CTL and all-Th constructs as well as intermixed arrangements of CTL and Th epitopes are also encompassed.

Another example of useful endosomal targeting signal is a portion (first 110 amino acids) or the whole sequence of the invariant chain (Ii) associated with MHC class II molecules. This signal enhances the efficiency of induction of CD4+ T-cell response. Also, Th epitopes may be associated with the immunoregulatory fragment of Ii, LRMKLPKPPKPVSQMR (SEQ ID NO: 17, Ii 77-92), or its shorter fragments such as, e.g., LRMKLPK (SEQ ID NO: 18) or LRMK (SEQ ID NO: 19).

N-terminally conjugated ubiquitin (e.g., ubiquitin with G76V substitution [UbV76]) can be used in the polyepitope constructs of the present invention to further enhance proteasomal processing of the epitopes contained in the constructs and also to enhance CTL (CD8+) responses. UbV76 can be conjugated directly to the amino terminus of the polyepitope construct or Val or Arg residue can be inserted between UbV76 and polyepitope construct to further stabilize the resulting chimeric constructs. See Example 2.4.5, below.

### 1.7 Production of the Polyepitope Constructs of the Invention

The polyepitope constructs of the present invention can be produced synthetically using various methods well known in the art (e.g., exclusive solid phase synthesis, automated solid phase synthesis, partial solid phase synthesis methods, fragment condensation, classical solution synthesis, etc.; see, e.g., Merrifield J. Am. Chem. Soc. 1963 85:2149 and Merrifield et al., 1982, Biochemistry, 21:502; Stewart, Solid Phase Peptide Syntheses, Freeman and Co.: San Francisco, 1969; 2002/2003 General Catalog from Novabiochem Corp, San Diego, USA; Goodman, Synthesis of Peptides and Peptidomimetics, Houben-Weyl, Stuttgart 2002) or can be expressed in a prokaryotic or eukaryotic host cell using various expression vectors encoding such constructs. Thus, provided herein are isolated polynucleotides that encode the polyepitope constructs of the present invention as well as recombinant vectors and host cells (both eukaryotic and prokaryotic) that have been genetically modified to express or overexpress the polyepitope constructs of the present invention. The host cells may be cultured or otherwise maintained under conditions permitting expression of the polyepitope polypeptide from the nucleic acid, e.g., the plasmid, encoding it.

The polyepitope constructs of the invention can be modified in various ways to improve their pharmacokinetic and other properties (e.g., to generate constructs with more favorable solubility, stability, and/or susceptibility to hydrolysis and/or proteolysis). Polyepitope constructs can be modified at the amino (N-) terminus, and/or carboxy (C-) terminus and/or by replacement of one or more of the naturally occurring genetically encoded amino acids with an unconventional amino acid, modification of the side chain of one or more amino acid residues, peptide phosphorylation, and the like.

Amino terminus modifications include methylation (e.g., —NHCH₃ or —N(CH₃)₂), acetylation (e.g., with acetic acid or a halogenated derivative thereof such as α-chloroacetic acid, α-bromoacetic acid, or α-iodoacetic acid), adding a benzyloxycarbonyl (Cbz) group, or blocking the amino terminus with any blocking group containing a carboxylate functionality defined by RCOO—or sulfonyl functionality defined by R—SO₂—, where R is selected from alkyl, aryl, heteroaryl, alkyl aryl, and the like, and similar groups. One can also incorporate a desamino acid at the N-terminus (so that there is no N-terminal amino group) to decrease susceptibility to proteases or to restrict the conformation of the peptide compound.

Carboxy terminus modifications include replacing the free acid with a carboxamide group or forming a cyclic lactam at the carboxy terminus to introduce structural constraints. One can also incorporate a desamino or descarboxy residue at the termini of the construct, so that there is no terminal amino or carboxyl group, to decrease susceptibility to proteases.

One can also replace any of the 20 naturally occurring amino acids. Common examples of conventional amino acid replacements include stereoisomers (e.g., D-amino acids) and unnatural amino acids such as, for example, L-ornithine, L-homocysteine, L-homoserine, L-citrulline, 3-sulfino-L-alanine, N-(L-arginino)succinate, 3,4-dihydroxy-L-phenylalanine, 3-iodo-L-tyrosine, 3,5-diiodo-L-tyrosine, triiodothyronine, L-thyroxine, L-selenocysteine, N-(L-arginino)taurine, 4-aminobutylate, (R,S)-3-amino-2-methylpropanoate, a,a-disubstituted amino acids, N-alkyl amino acids, lactic acid, β-alanine, 3-pyridylalanine, 4-hydroxyproline, O-phosphoserine, N-methylglycine, N-acetylserine, N-formylmethionine, 3-methylhistidine, 5-hydroxylysine, nor-leucine, and other similar amino acids and imino acids. A general method for site-specific incorporation of unnatural amino acids into proteins and peptides is described in Noren et al., Science, 244:182-188 (April 1989).

### 1.8 Pharmaceutical and Immunogenic Compositions and Methods for Delivery of the Polyepitope Constructs of the Invention

The polyepitope constructs of the invention can be administered directly, but are preferably administered as part of immunogenic compositions comprising pharmaceutically acceptable carrier(s) and/or excipient(s). In a specific embodiment, the polyepitope constructs of the invention are administered conjointly (together in one composition or separately in two different compositions, which can be administered simultaneously or sequentially to the same or different site) with an adjuvant. Any adjuvant known in the art can be used. Non-limiting examples of adjuvants useful in the immunogenic compositions of the present invention include oil-emulsion and emulsifier-based adjuvants such as complete Freund's adjuvant, incomplete Freund's adjuvant, AS03, MF59, or SAF; mineral gels such as aluminum hydroxide (alum), aluminum phosphate or calcium phosphate; microbially-derived adjuvants such as cholera toxin (CT), pertussis toxin, Escherichia coli heat-labile toxin (LT), mutant toxins (e.g., LTK63 or LTR72), Bacille Calmette-Guerin (BCG), Corynebacterium parvum, DNA CpG motifs, muramyl dipeptide, or monophosphoryl lipid A; particulate adjuvants such as immunostimulatory complexes (ISCOMs), liposomes, biodegradable microspheres, or saponins (e.g., QS-21); cytokines such as IFN-γ, IL-2, IL-12 or GM-CSF; synthetic adjuvants such as nonionic block copolymers, muramyl peptide analogues (e.g., N-acetyl-muramyl-L-threonyl-D-isoglutanine [thr-MDP], N-acetyl-nor-muramyl-L-alanyl-D-isoglutamine, N-acetylmuramyl-L-alanyl-D-isoglutaminyl-L-alanine-2-[1′-2′-dipalmitoyl-s-n-glycero-3-hydroxyphosphoryloxy]-ethylamine), polyphosphazenes, or synthetic polynucleotides, and surface active substances such as lysolecithin, pluronic polyols, polyanions, peptides, hydrocarbon emulsions, or keyhole limpet hemocyanins (KLH). Preferably, these adjuvants are pharmaceutically acceptable for use in humans.

The polyepitope constructs of the invention can be also administered in the form of nucleic acids encoding such polyepitope constructs (e.g., a plasmid, viral or any other appropriate vector). To achieve expression of the polyepitope construct in a target cell (e.g., dendritic cell (DC), Langerhans cell, or other antigen presenting cell (APC), or any other host cell), such vectors should contain one or more regulatory sequences which permit expression in such cells. Such regulatory sequence(s) can be operatively linked to the sequence encoding the polyepitope construct, such that they drive expression of the latter.

The polyepitope constructs of the invention or nucleic acids encoding them can be delivered in a microparticle that also includes a polymeric matrix or in a synthetic viral vector. Any suitable viral vector can be used (e.g., Adenovirus, Poxvirus, Lentivirus, etc.). See also http://www.ncbi.nlm.nih.gov/sites/entrez?db=pubmed&cmd=DetailsSearch&term=microparticle+polymeric+antigen.

When the polyepitope constructs of the invention are administered as nucleic acids and/or using various delivery vehicles (e.g., microparticles, virus-like particles), such nucleic acids and/or delivery vehicles can further enhance the antigen-specific immune responses (e.g., by promoting IL-12 and γ-interferon (IFN) release from macrophages, NK cells, and T cells).

The polyepitope constructs of the invention can be used to produce antigen presenting cells (APCs, e.g., dendritic cells (DC), Langerhans cells, or other type), capable to present desired epitopes to the lymphocytes. Desired APCs can be obtained using any method known in the art, e.g., in vitro by transfecting e.g. DCs (derived from e.g. monocytes of the patient) with polynucleotides (either DNA or mRNA), coding for the polyepitope, or by pulsing with corresponding polyepitope polypeptide, or by infecting with recombinant vector microorganism bearing corresponding gene coding for the polyepitope, or some other similar technique known in the art. Produced APCs can be used either as a therapeutic cellular vaccine, or to produce ex vivo autologous effector T-cells for using them as a therapeutic cellular vaccine.

The polypeptide and nucleic acid constructs and compositions of the invention can be administered via different routes. For example, they can be administered to mucosal tissue (e.g., vaginal, nasal, lower respiratory, or gastrointestinal tissue [e.g., rectal]). Alternatively, they can be administered systemically, for example, intravenously, intramuscularly, intradermally, orally, or subcutaneously.

### 1.9 Effective Dose and Safety Evaluations

According to the methods of the present invention, the pharmaceutical and immunogenic compositions described herein are administered to a patient at immunogenically effective doses, preferably, with minimal toxicity.

Following methodologies which are well-established in the art (see, e.g., Goldenthal et al., National Cooperative Vaccine Development Working Group. AIDS Res. Hum. Retroviruses 1993, 9:545-9), effective doses and toxicity of the compounds and compositions of the instant invention can be first determined in preclinical studies using small animal models (e.g., mice) in which these compounds and compositions can be reproducibly immunized by the same route proposed for the human clinical trials. Specifically, for any pharmaceutical composition or vaccine used in the methods of the invention, the therapeutically effective dose can be estimated initially from animal models to achieve a circulating plasma concentration range that includes the IC50 (i.e., the concentration of the test compound which achieves a half-maximal inhibition of symptoms). Dose-response curves derived from animal systems are then used to determine testing doses for the initial clinical studies in humans. In safety determinations for each composition, the dose and frequency of immunization should meet or exceed those anticipated for use in the clinical trial.

As disclosed herein, the dose of polyepitope constructs and other components in the compositions of the present invention is determined to ensure that the dose administered continuously or intermittently will not exceed a certain amount in consideration of the results in test animals and the individual conditions of a patient. A specific dose naturally varies depending on the dosage procedure, the conditions of a patient or a subject animal such as age, body weight, sex, sensitivity, feed, dosage period, drugs used in combination, seriousness of the disease. The appropriate dose and dosage times under certain conditions can be determined by the test based on the above-described indices and should be decided according to the judgment of the practitioner and each patients circumstances according to standard clinical techniques. In this connection, the preferred dose of a polyepitope construct is generally in the range of 1-950 μg per kg of the body weight depending on the mode of delivery and immunization.

Toxicity and therapeutic efficacy of polyepitope constructs in immunogenic compositions of the invention can be determined by standard pharmaceutical procedures in experimental animals, e.g., by determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compositions that exhibit large therapeutic indices are preferred. While therapeutics that exhibit toxic side effects can be used (e.g., when treating severe forms of cancer, life-threatening infections or autoimmune diseases), care should be taken to design a delivery system that targets such immunogenic compositions to the specific site in order to minimize potential damage to other tissues and organs and, thereby, reduce side effects. As disclosed herein, the polyepitope constructs of the invention are highly immunostimulating and possess low toxicity.

As specified above, the data obtained from the animal studies can be used in formulating a range of dosage for use in humans. The therapeutically effective dosage of polyepitope constructs of the present invention for use in humans lies preferably within a range of circulating concentrations that include the ED50 with little or no toxicity. The dosage can vary within this range depending upon the dosage form employed and the route of administration utilized. Ideally, a single dose should be used.

### 2 EXAMPLES

The present invention is further described by way of the following particular examples. However, the use of such examples is illustrative only and is not intended to limit the scope or meaning of this invention or of any exemplified term. Nor is the invention limited to any particular preferred embodiment(s) described herein. Indeed, many modifications and variations of the invention will be apparent to those skilled in the art upon reading this specification, and such “equivalents” can be made without departing from the invention in spirit or scope. The invention is therefore limited only by the terms of the appended claims, along with the full scope of equivalents to which the claims are entitled.

2.0 Sequence of Human Full-Length ErbB2 (HER2) Protein:

>gi|119533|sp|P04626.1|ERBB2_HUMAN RecName: Full = Receptor tyrosine-protein
kinase erbB-2; AltName: Full = p185erbB2; AltName: Full = C-erbB-2; AltName:
Full = NEU proto-oncogene; AltName: Full = Tyrosine kinase-type cell surface
receptor HER2; AltName: Full = MLN 19; AltName: CD_antigen = CD340; Flags: Precursor
(SEQ ID NO: 20)
MELAALCRWGLLLALLPPGAASTQVCTGTDMKLRLPASPETHLDMLRHLYQGCQVVQGNLELTYLPTNAS
LSFLQDIQEVQGYVLIAHNQVRQVPLQRLRIVRGTQLFEDNYALAVLDNGDPLNNTTPVTGASPGGLREL
QLRSLTEILKGGVLIQRNPQLCYQDTILWKDIFHKNNQLALTLIDTNRSRACHPCSPMCKGSRCWGESSE
DCQSLTRTVCAGGCARCKGPLPTDCCHEQCAAGCTGPKHSDCLACLHFNHSGICELHCPALVTYNTDTFE
SMPNPEGRYTFGASCVTACPYNYLSTDVGSCTLVCPLHNQEVTAEDGTQRCEKCSKPCARVCYGLGMEHL
REVRAVTSANIQEFAGCKKIFGSLAFLPESFDGDPASNTAPLQPEQLQVFETLEEITGYLYISAWPDSLP
DLSVFQNLQVIRGRILHNGAYSLTLQGLGISWLGLRSLRELGSGLALIHHNTHLCFVHTVPWDQLFRNPH
QALLHTANRPEDECVGEGLACHQLCARGHCWGPGPTQCVNCSQFLRGQECVEECRVLQGLPREYVNARHC
LPCHPECQPQNGSVTCFGPEADQCVACAHYKDPPFCVARCPSGVKPDLSYMPIWKFPDEEGACQPCPINC
THSCVDLDDKGCPAEQRASPLTSIISAVVGILLVVVLGVVFGILIKRRQQKIRKYTMRRLLQETELVEPL
TPSGAMPNQAQMRILKETELRKVKVLGSGAFGTVYKGIWIPDGENVKIPVAIKVLRENTSPKANKEILDE
AYVMAGVGSPYVSRLLGICLTSTVQLVTQLMPYGCLLDHVRENRGRLGSQDLLNWCMQIAKGMSYLEDVR
LVHRDLAARNVLVKSPNHVKITDFGLARLLDIDETEYHADGGKVPIKWMALESILRRRFTHQSDVWSYGV
TVWELMTFGAKPYDGIPAREIPDLLEKGERLPQPPICTIDVYMIMVKCWMIDSECRPRFRELVSEFSRMA
RDPQRFVVIQNEDLGPASPLDSTFYRSLLEDDDMGDLVDAEEYLVPQQGFFCPDPAPGAGGMVHHRHRSS
STRSGGGDLTLGLEPSEEEAPRSPLAPSEGAGSDVFDGDLGMGAAKGLQSLPTHDPSPLQRYSEDPTVPL
PSETDGYVAPLTCSPQPEYVNQPDVRPQPPSPREGPLPAARPAGATLERPKTLSPGKNGVVKDVFAFGGA
VENPEYLTPQGGAAPQPHPPPAFSPAFDNLYYWDQDPPERGAPPSTFKGTPTAENPEYLGLDVPV

The following Examples illustrate the invention without limiting its scope.

2.1 Universal CTL Epitopes

2.1.1 A List of Universal CTL Epitopes

(SEQ ID NOS: 21, 22, 23, 24, 25, 26, 27, 28, 29,
30, 31, 32, 33, 34, 35, 36, 37, 38, 39, 40, 41,
42, 43, 44, 45, 46, 47, 48, 49, 50, 51, 52, 53,
54, 55, 56, 57, 58, 59, 60, 61, 62, 63, 64, 65,
66, 67, 68, 69, 70, 71, 72, 73, 74, 75, 76, and
77, respectively)
CRWGLLLAL,
LAALCRWGL,
RELGSGLAL,
WGLLLALLP,
LVVVLGVVF,
KITDFGLAR,
QLFEDNYAL,
YISAWPDSL,
GDLTLGLEP,
DVWSYGVTV,
KIFGSLAFL,
FDGDLGMGA,
LVHRDLAAR,
MELAALCRW,
RASPLTSII,
RGAPPSTFK,
SIISAVVGI,
LHCPALVTY,
LRIVRGTQL,
VKVLGSGAF,
LQPEQLQVF,
VKIPVAIKV,
QLMPYGCLL,
QETELVEPL,
DIFHKNNQL,
ASCVTACPY,
TELVEPLTP,
PLQRLRIVR,
LQVIRGRIL,
DEAYVMAGV,
EECRVLQGL,
TVCAGGCAR,
YSEDPTVPL,
RWGLLLALL,
FEDNYALAV,
QEVQGYVLI,
LLALLPPGA,
GSGAFGTVY,
LGTSWLGLR,
ISAVVGILL,
MQIAKGMSY,
LSYMPIWKF,
GVVKDVFAF,
AIKVLRENT,
SWLGLRSLR,
ILLVVVLGV,
FGPEADQCV,
TLQGLGISW,
TDFGLARLL,
DSTFYRSLL,
IISAVVGIL,
TTPVTGASP,
GMEHLREVR,
ALCRWGLLL,
RIVRGTQLF,
GSCTLVCPL,
DGENVKIPV

2.1.2 A List of Peptide Fragments Containing Overlapping ErbB2 Epitopes

(SEQ ID NOS: 78, 56, 79, 72, 45, 52, 38, 46, 76,
73, 31, 41, 28, 49, 80, 51, 67 62, 81, 82, 83,
84, 50, 43, 61, 33, 85, 30. 70, 29, 32, 53, 63,
and 36, respectively)
MELAALCRWGLLLALLPPGA,
QEVQGYVLI,
PLQRLRIVRGTQLFEDNYALAV,
TTPVTGASP,
DIFHKNNQL,
TVCAGGCAR,
LHCPALVTY,
ASCVTACPY,
GSCTLVCPL,
GMEHLREVR,
KIFGSLAFL,
LQPEQLQVF,
YISAWPDSL,
LQVIRGRIL,
TLQGLGISWLGLRSLRELGSGLAL,
EECRVLQGL,
FGPEADQCV,
LSYMPIWKF,
RASPLTSIISAVVGILLVVVLGVVF,
QETELVEPLTP,
VKVLGSGAFGTVY,
DGENVKIPVAIKVLRENT,
DEAYVMAGV,
QLMPYGCLL,
MQIAKGMSY,
LVHRDLAAR,
KITDFGLARLL,
DVWSYGVTV,
DSTFYRSLL,
GDLTLGLEP,
FDGDLGMGA,
YSEDPTVPL,
GVVKDVFAF,
RGAPPSTFK

2.1.3 Several Versions of Universal PolyCTL Constructs

Constructs with fixed optimal spacer sequence:

For 33 peptides selected with 3-fold excess (overall sequence length 297 aa):

2.1.3.1 polyCTL Construct with Spacer Sequences which Optimize TAP Interaction and Proteasome Processing:

(SEQ ID NO: 86)
CRWGLLLALLVVVLGVVFSIISAVVGIRELGSGLALMELAALCRWADLAR
DEAYVMAGVADLVEECRVLQGLADYSEDPTVPLAVKIPVAIKVAQLFEDN
YALADVWSYGVTVAWGLLLALLPATVCAGGCARADIFHKNNQLADASCVT
ACPYADLLHCPALVTYATELVEPLTPADLKITDFGLARARGAPPSTFKAD
LYISAWPDSLAQETELVEPLALQVIRGRILALAALCRWGLADLQLMPYGC
LLADKIFGSLAFLARGDLTLGLEPAVKVLGSGAFADLVHRDLAARADLQP
EQLQVFADAFDGDLGMGAAPLQRLRIVRADLRIVRGTQLARASPLTSII

(overall length is 349 aa; spacers constitute 17.5% of the sequence)

2.1.3.2. polyCTL Construct with Spacer Sequences which Optimize TAP Interaction and Immunoproteasome Processing:

(SEQ ID NO: 87)
QETELVEPLASCVTACPYADLVKVCRWGLLLALSIISAVVGIAARDEAYV
MAGVADLVKLHCPALVTYARASPLTSIIADLVEECRVLQGLAFDGDLGMG
AARGAPPSTFKADLKIFGSLAFLMELAALCRWADLVQLMPYGCLLAQLFE
DNYALKITDFGLARADYISAWPDSLTVCAGGCARADLWGLLLALLPADLV
HRDLAARADLYSEDPTVPLRELGSGLALARGDLTLGLEPAVKVLGSGAFA
DLQPEQLQVFADLDVWSYGVTVADLRIVRGTQLAPLQRLRIVRADLAALC
RWGLAVKIPVAIKVADLQVIRGRILALVVVLGVVFADIFHKNNQLATELV
EPLTP

(overall length is 355 aa; spacers constitute 19.5% of the length)

2.1.3.3. polyCTL Construct with Spacer Sequences which Optimize TAP Interaction and Proteasome and Immunoproteasome Processing:

(SEQ ID NO: 88)
CRWGLLLALASCVTACPYADLYISAWPDSLAVKIPVAIKVAQLFEDNYAL
ADVWSYGVTVAWGLLLALLPADIFHKNNQLATELVEPLTPADLLHCPALV
TYAPLQRLRIVRADLQLMPYGCLLADKIFGSLAFLMELAALCRWADLVHR
DLAARADLQPEQLQVFADAFDGDLGMGAALQVIRGRILAVKVLGSGAFAD
LRIVRGTQLARGAPPSTFKADLQETELVEPLRELGSGLALLVVVLGVVFS
IISAVVGIARGDLTLGLEPADKITDFGLARALAALCRWGLADYSEDPTVP
LTVCAGGCARARASPLTSIIADLVEECRVLQGLAARDEAYVMAGV

(overall length is 345 aa; spacers constitute 16.1% of the length)

For 57 peptides selected with 5-fold excess:

2.1.3.4. polyCTL Construct with Spacer Sequences which Optimize TAP Interaction and Proteasome Processing:

(SEQ ID NO: 89)
CRWGLLLALAFGPEADQCVADLQLMPYGCLLADYSEDPTVPLAVKIPVAI
KVAQLFEDNYALADVWSYGVTVAWGLLLALLPATVCAGGCARAISAVVGI
LLATLQGLGISWADSWLGLRSLRADLVKRWGLLLALLLLALLPPGARELG
SGLALLVVVLGVVFSIISAVVGIILLVVVLGVAIISAVVGILAIKVLREN
TADLVQETELVEPLALQVIRGRILAGVVKDVFAFADLARDEAYVMAGVAD
LPLQRLRIVRADLKITDFGLARALGISWLGLRADLQEVQGYVLIADLHCP
ALVTYAVKVLGSGAFADGMEHLREVRADTTPVTGASPADASCVTACPYAD
LYISAWPDSLARGDLTLGLEPADRGAPPSTFKADLRIVRGTQLATELVEP
LTPADAFDGDLGMGAALAALCRWGLADLQPEQLQVFADAFEDNYALAVAM
QIAKGMSYATDFGLARLLMELAALCRWADLVHRDLAARADGSGAFGTVYA
RDGENVKIPVADLVDSTFYRSLLADLVEECRVLQGLADKIFGSLAFLALC
RWGLLLADIFHKNNQLADLSYMPIWKFADLVGSCTLVCPLARASPLTSII
ADLRIVRGTQLF

(overall length is 612 aa; spacers constitute 19.3% of the length)

2.1.3.5. polyCTL Construct with Spacer Sequences which Optimize TAP Interaction and Immunoproteasome Processing:

(SEQ ID NO: 90)
TTPVTGASPADLSWLGLRSLRADLVGSCTLVCPLAIKVLRENTADYSEDP
TVPLMELAALCRWADLRWGLLLALLILLVVVLGVADLWGLLLALLPADLV
HRDLAARADLDVWSYGVTVADLGISWLGLRADLVKVQETELVEPLTDFGL
ARLLRELGSGLALAIISAVVGILAFGPEADQCVADLVKVCRWGLLLALIS
AVVGILLGSGAFGTVYADLSYMPIWKFADLVEECRVLQGLGVVKDVFAFA
DLAFEDNYALAVADLKIFGSLAFLASCVTACPYADLVKVQLMPYGCLLAA
RDEAYVMAGVADLVKLHCPALVTYAVKVLGSGAFADLQPEQLQVFADLRI
VRGTQLFADLVDSTFYRSLLADGMEHLREVRADLRIVRGTQLATVCAGGC
ARADLAALCRWGLAPLQRLRIVRADLQVIRGRILALVVVLGVVFADIFHK
NNQLATLQGLGISWAQLFEDNYALARGDLTLGLEPAARDGENVKIPVADL
VALCRWGLLLALLALLPPGAARGAPPSTFKADLKITDFGLARADMQIAKG
MSYADAFDGDLGMGAAVKIPVAIKVARASPLTSIIADLQEVQGYVLIADY
ISAWPDSLSIISAWGIATELVEPLTP

(overall length is 627 aa; spacers constitute 22.2% of the length)

2.1.3.6. polyCTL Construct with Spacer Sequences which Optimize TAP Interaction and Proteasome and Immunoproteasome Processing:

(SEQ ID NO: 91)
CRWGLLLALISAVVGILLAFGPEADQCVADLQETELVEPLTDFGLARLLR
ELGSGLALLVVVLGVVFSIISAVVGIILLVVVLGVAIISAVVGILGSGAF
GTVYAIKVLRENTADLRIVRGTQLFADLVKLHCPALVTYAVKVLGSGAFA
DGMEHLREVRADYISAWPDSLALCRWGLLLAVKIPVAIKVALAALCRWGL
ADTTPVTGASPADRGAPPSTFKADLYSEDPTVPLAFDGDLGMGALLALLP
PGAARDGENVKIPVADLVDSTFYRSLLADGSCTLVCPLMELAALCRWADS
WLGLRSLRADLVPLQRLRIVRADLKITDFGLARALGISWLGLRADLQEVQ
GYVLIADKIFGSLAFLASCVTACPYADLRASPLTSIIADLVEECRVLQGL
AARDEAYVMAGVADLRWGLLLALLGVVKDVFAFADLQLMPYGCLLADLQP
EQLQVFADLRIVRGTQLAMQIAKGMSYADVWSYGVTVAWGLLLALLPATV
CAGGCARAQLFEDNYALARGDLTLGLEPADIFHKNNQLATELVEPLTPAD
LVHRDLAARADAFEDNYALAVALQVIRGRILATLQGLGISWADLSYMPIW
KF

(overall length is 602 aa; spacers constitute 17.3% of the length)

### Using Degenerate Spacer Sequence:

2.1.3.7. Before selection of spacer sequences for optimal proteasome processing of 57 selected epitopes, selection of spacers optimal for TAP interaction was conducted. Selection was optimized to minimize the number of junk epitopes and to maximize the number of interacting MHC I alleles, keeping the spacer sequences of the minimal size as preferred.

(SEQ ID NO: 92)
TVCAGGCARADGMEHLREVRADGKEECRVLQGLADGRELGSGLALPQLFE
DNYALSDGQETELVEPLPLVVVLGVVFARDGENVKIPVALLALLPPGAAQ
EVQGYVLIPDLARGDLTLGLEPAIKVLRENTADAFDGDLGMGAPDAKARD
EAYVMAGVADIFHKNNQLAVKVLGSGAFATLQGLGISWAIAFGPEADQCV
PDLKLSYMPIWKFADLKPLQRLRIVRAIISAVVGILMELAALCRWATGVV
KDVFAFADLVKIPVAIKVSIISAVVGIPISAVVGILLPILQPEQLQVFAD
GKYSEDPTVPLADMQIAKGMSYARGAPPSTFKADLQVIRGRILPDGRASP
LTSIIADLVHRDLAARADSWLGLRSLRADGKLGISWLGLRADGVKITDFG
LARATDFGLARLLPDGDSTFYRSLLAILLVVVLGVADTTPVTGASPRDLR
IVRGTQLATELVEPLTPPDLKASCVTACPYPILAALCRWGLADAFEDNYA
LAVAIDVWSYGVTVAWGLLLALLPRDAKQLMPYGCLLAIKIFGSLAFLAL
CRWGLLLRDGRIVRGTQLFADLVGSGAFGTVYADGGSCTLVCPLPDGYIS
AWPDSLRDLHCPALVTYALLVCRWGLLLALRWGLLLALL

(the overall length is 639 aa with spacer sequences constituting 22% of the overall length; in this construct, with chosen stringency of proteasome filter, 29 junk epitopes were predicted keeping all predicted epitopes; spacer sequences are underlined)

2.1.3.8. Before selection of spacer sequences for optimal proteasome processing of 34 selected overlapping epitopes, selection of spacers optimal for TAP interaction was conducted. Selection was optimized to minimize the number of junk epitopes and to maximize the number of interacting MHC I alleles:

(SEQ ID NO: 93)
MELAALCRWGLLLALLPPGAPDGENVKIPVAIKVLRENTADGKEECRVLQ
GLPDGKYSEDPTVPLPDDEAYVMAGVADLKQETELVEPLTPPDGRASPLT
SIISAVVGILLVVVLGVVFPDAGMEHLREVRADGKDIFHKNNQLPDLQPE
QLQVFRDAQEVQGYVLIPDLAFDGDLGMGAPDLQVIRGRILPDVKVLGSG
AFGTVYPIGDLTLGLEPPDLKASCVTACPYATLQGLGISWLGLRSLRELG
SGLALPMQIAKGMSYALFGPEADQCVPDLKLSYMPIWKFADLKPLQRLRI
VRGTQLFEDNYALAVARGAPPSTFKAGVVKDVFAFRDLVKITDFGLARLL
PLVHRDLAARADVWSYGVTVRDTTPVTGASPRDLYISAWPDSLRTVCAGG
CARSDKIFGSLAFLPDLHCPALVTYADDSTFYRSLLADGKQLMPYGCLLA
DGGSCTLVCPL

(the overall length is 461 aa with spacer sequences constituting 22% of the overall length; in this construct, with chosen stringency of proteasome filter, 18 initially chosen epitopes are not predicted, but there are only 9 junk epitopes not present in ErbB2; with minimal stringency of proteasome filter, only 7 initially chosen epitopes are not predicted, but the number of junk epitopes increases to 106; spacer sequences are underlined)

2.2 Allele-Specific CTL Epitopes

2.2.1 Table of Chosen Allele-Specific Epitopes and Polyepitope Constructs

| HLA allele | Peptides | Example of poly CTL construct(s) |
|---|---|---|
| A*0101 | LTCSPQPEY, GSGAFGTVY, | WGLLLALLP-RDA-YSEDPTVPL-ADIDETEYHA-PDLK- |
| | EGAGSDVFD, YKDPPFCVA, | AREEGAGSDVFD-AYGVTVWELM-ALGK-ARDDDDMGDLVD- |
| | TIDVYMIMV, YGVTVWELM, | PLGK-AEITGYLYIS-ADGK-HLDMLRHLY-ADLK- |
| | DGENVKIPV, LLDIDETEY, | AHSDCLACLH-AD-LTCSPQPEY-ADLK-QSDVWSYGV-AD- |
| | QSDVWSYGV, HLDMLRHLY, | AYKDPPFCVA-PDL-ARDGDLGMGAA-PIAK-LLDIDETEY- |
| | DGDPASNTA, NASLSFLQD, | AD-ARDGDPASNTA-AI-ARDGENVKIPV-ALL- |
| | DGDLGMGAA, FSPAFDNLY, | GSGAFGTVY-PD-NASLSFLQD-PLLK-LHCPALVTY-AD- |
| | DSTFYRSLL, WGLLLALLP, | DSTFYRSLL-ADL-FSPAFDNLY-AILK-TIDVYMIMV |
| | YSEDPTVPL, LHCPALVTY, | (SEQ ID NO: 110) |
| | EITGYLYIS, DDDMGDLVD, | |
| | HSDCLACLH, DIDETEYHA | |
| | (SEQ ID NOS: . . . 94, 58, | |
| | 95, 96, 97, 98, 77, | |
| | 99, 100, 101, 102, | |
| | 103, 104, 105, 70, 24, | |
| | 53, 38, 106, 107, 108, | |
| | and 109, respectively) | |
| A*0201 | LLLALLPPG, ILDEAYVMA, | Var1: TIDVYMIMV-PDLK-CRWGLLLAL-A- |
| | ILHNGAYSL, RLLQETELV, | LLALLPPGA-ADG-AILDEAYVMA-ALIHHNTHL-PDL- |
| | CRWGLLLAL, TIDVYMIMV, | RLVHRDLAA-LLLALLPPG-ADGK-QLFEDNYAL-P- |
| | MIMVKCWMI, LVDAEEYLV, | ILHNGAYSL-P-SLTLQGLGI-R-LVDAEEYLV-R- |
| | RLVHRDLAA, ALCRWGLLL, | ILLVVVLGV-ADA-SIISAVVGI-A-RLLQETELV-AD- |
| | LLNWCMQIA, ALIHHNTHL, | AFEDNYALAV-AVVGILLVV-A-VVLGVVFGI-AD- |
| | LLALLPPGA, QLFEDNYAL, | ALLNWCMQIA-ADLV-ALCRWGLLL-AD-YISAWPDSL-RD- |
| | AVVGILLVV, KIFGSLAFL, | KIFGSLAFL-RDL-QLMPYGCLL-ADG-MIMVKCWMI |
| | QLMPYGCLL, FEDNYALAV, | (SEQ ID NO: 123) |
| | VVLGVVFGI, ILLVVVLGV, | Var2: |
| | SIISAVVGI, SLTLQGLGI, | MELAALCRWGLLLALLPPGAPPDLLALLPPGAPDATLEEITG |
| | YISAWPDSL | YLAILDEAYVMAPILHNGAYSLPQLFEDNYALSIISAVVGIA |
| | (SEQ ID NOS: 111,112, | QLMPYGCLLRLLVVVLGVVRDLQLRSLTEIAILLVVVLGVPD |
| | 113, 114, 21, 97, 115, | AWGILLVVADALCRWGLLLADYISAWPDSLRDKIFGSLAFL |
| | 116, 117, 74, 118, | (SEQ ID NO: 124) |
| | 119, 57, 27, 120, 31, | |
| | 43, 55, 121, 66, 37, | |
| | 122, and 28 . . . , | |
| | respectively) | |
| A*0202 | CLTSTVQLV, ILDEAYVMA, | LVPQQGFFC-ADLV-PCARVCYGL-PDLK-KHSDCLACL-- |
| | ILHNGAYSL, QIAKGMSYL, | ATLEEITGYL-A-TLSPGKNGV-PDL-DLVDAEEYL-P- |
| | PCARVCYGL, RLLQETELV, | ILHNGAYSL-A-SLPDLSVFQ-RD-QIAKGMSYL- |
| | KHSDCLACL, MIMVKCWMI, | AILDEAYVMA-ALIHHNTHL-AI-AFGPEADQCV-RDLK- |
| | RWGLLLALL, TYLPTNASL, | LVDAEEYLV-A-QLFEDNYAL-SIISAVVGI-ADG- |
| | LVDAEEYLV, SLPDLSVFQ, | THLDMLRHL-ACLTSTVQLV-ADG-FRNPHQALL-ADG- |
| | FRNPHQALL, TLEEITGYL, | RLLQETELV-ADL-KIFGSLAFL-A-YISAWPDSL-RD- |
| | DLVDAEEYL, ALIHHNTHL, | AYSLTLQGL-RDL-TYLPTNASL-SDA-RWGLLLALL-A- |
| | QLFEDNYAL, AYSLTLQGL, | QLMPYGCLL-ADG-MIMVKCWMI |
| | KIFGSLAFL, QLMPYGCLL, | (SEQ ID NO: 138) |
| | YISAWPDSL, FGPEADQCV, | |
| | LVPQQGFFC, SIISAVVGI, | |
| | THLDMLRHL, TLSPGKNGV | |
| | (SEQ ID NOS: 125, 112, | |
| | 113, 126, 127, 114, | |
| | 128, 115, 54, 129, | |
| | 116, 130, 131, 132, | |
| | 133, 119, 27, 134, 31, | |
| | 43, 28, 67, 135, 37, | |
| | 136, and 137, | |
| | respectively) | |
| A*0203 | HYKDPPFCV, CLTSTVQLV, | HYKDPPFCV-AIGK-AIQNEDLGPA-RDL-QIAKGMSYL-A- |
| | YLTPQGGAA, QIAKGMSYL, | TLSPGKNGV-SD-LLALLPPGA-ADG-PYVSRLLGI- |
| | SLRELGSGL, HLYQGCQVV, | AYLSTDVGSC-AD-ILLVVVLGV-ADA-SIISAVVGI-AD- |
| | MIMVKCWMI, PLTSIISAV, | SLRELGSGL-PTG-RASPLTSII-A-LLVVVLGVV-RDL- |
| | PYVSRLLGI, FRNPHQALL, | AYLTPQGGAA-ALIHHNTHL-AD-ARPLTSIISAV-ADL- |
| | RASPLTSII, ILLVVVLGV, | FRNPHQALL-ADGK-KIFGSLAFL-ALLNWCMQIA-ADLK- |
| | LLNWCMQIA, ALIHHNTHL, | ACLTSTVQLV-ADG-YISAWPDSL-A-HLYQGCQVV-ADL- |
| | LLALLPPGA, IQNEDLGPA, | SLTLQGLGI-AD-QLMPYGCLL-ADG-MIMVKCWMI |
| | KIFGSLAFL, YLSTDVGSC, | (SEQ ID NO: 148) |
| | QLMPYGCLL, LLVVVLGVV, | |
| | TLSPGKNGV, SIISAVVGI, | |
| | SLTLQGLGI, YISAWPDSL | |
| | (SEQ ID NOS: 139, 125, | |
| | 140, 126, 141, 142, | |
| | 115, 143, 144, 131, | |
| | 35, 66, 118, 119, 57, | |
| | 145, 31, 146, 43, 147, | |
| | 137, 37, 122, and 28 . . . , | |
| | respectively) | |
| A*0206 | QVFETLEEI, LQLRSLTEI, | CRWGLLLAL-PD-AIQNEDLGPA-AVLDNGDPL- |
| | YVLIAHNQV, QIAKGMSYL, | RLLQETELV-ADG-FRNPHQALL-PDLK-QVFETLEEI-PD- |
| | LLVVVLGVV, RLLQETELV, | QIAKGMSYL-PD-VVLGVVFGI-ADA-TQLFEDNYA-AD- |
| | CRWGLLLAL, TIDVYMIMV, | AVVGILLVV-AD-RASPLTSII-A-LLVVVLGVV-RD- |
| | MIMVKCWMI, LAALCRWGL, | LQLRSLTEI-A-ILLVVVLGV-ADA-SIISAVVGI-PD- |
| | AVLDNGDPL, FRNPHQALL, | YVLIAHNQV-AD-VKIPVAIKV--ALIHHNTHL-A- |
| | RASPLTSII, ALIHHNTHL, | LAALCRWGL-A-SAVVGILLV-ADGK-KIFGSLAFL-A- |
| | IWIPDGENV, TQLFEDNYA, | IWIPDGENV-AD-TIDVYMIMV-QLMPYGCLL-ADG- |
| | SAVVGILLV, IQNEDLGPA, | MIMVKCWMI |
| | AVVGILLVV, KIFGSLAFL, | (SEQ ID NO: 156) |
| | QLMPYGCLL, VKIPVAIKV, | |
| | VVLGVVFGI, ILLVVVLGV, | |
| | SIISAVVGI | |
| | (SEQ ID NOS: 149, 150, | |
| | 151, 126, 147, 114, | |
| | 21, 97, 115, 22, 152, | |
| | 131, 35, 119, 153, | |
| | 154, 155, 145, 120, | |
| | 31, 43, 42, 121, 66, | |
| | and 37, respectively) | |
| A*0301 | LAARNVLVK, VVFGILIKR, | CVNCSQFLR-AD-LVKSPNHVK-A-ILKETELRK-RDLK- |
| | VMAGVGSPY, RILHNGAYS, | ARILHNGAYS-AD-GVVFGILIK-ADG-AELMTFGAKP- |
| | LLLALLPPG, TFYRSLLED, | PDGK-LELTYLPTN-ALGK-KIRKYTMRR-ADLV- |
| | VVVLGVVFG, QLVTQLMPY, | LERPKTLSP-A-VLRENTSPK-A-LLLALLPPG-ADGK- |
| | GILLVVVLG, LELTYLPTN, | RSLTEILKG-ALLHTANRP-A-ILIKRRQQK-ADGK- |
| | LVKSPNHVK, ELMTFGAKP, | AGILLVVVLG-PDGK-TVWELMTFG-A-ILWKDIFHK- |
| | TVWELMTFG, YLYISAWPD, | ADGK-RGAPPSTFK-ADL-QLVTQLMPY-A-VVVLGVVFG- |
| | ILKETELRK, YTMRRLLQE, | PD-VMAGVGSPY-AILK-LAARNVLVK-ADL-YTMRRLLQE- |
| | RSLTEILKG, GVVFGILIK, | ADGK-TFYRSLLED-RD-VVFGILIKR-A-LAFLPESFD-A- |
| | VLRENTSPK, CVNCSQFLR, | YLYISAWPD-AD-MTFGAKPYD |
| | ILIKRRQQK, LERPKTLSP, | (SEQ ID NO: 183) |
| | MTFGAKPYD, ALLHTANRP, | |
| | KIRKYTMRR, RGAPPSTFK, | |
| | ILWKDIFHK, LAFLPESFD | |
| | (SEQ ID NOS: . . . 157, | |
| | 158, 159, 160, 111, | |
| | 161, 162, 163, 164, | |
| | 165, 166, 167, 168, | |
| | 169, 170, 171, 172, | |
| | 173, 174, 175, 176, | |
| | 177, 178, 179, 180, | |
| | 36, 181, and 182, | |
| | respectively) | |
| A*2301 | VYMIMVKCW, DVWSYGVTV, | RWGLLLALL-A-EYVNARHCL-R-DLLEKGERL- |
| | RWGLLLALL, DIFHKNNQL, | AEYHADGGKV-S-DIFHKNNQL-A-QLFEDNYAL-P- |
| | SYGVTVWEL, MIMVKCWMI, | LAALCRWGL-AI-AYGVTVWELM-AI-LRIVRGTQL- |
| | EYLVPQQGF, TYLPTNASL, | ILLVVVLGV-ADA-TYLPTNASL-A-IWIPDGENV-RLL- |
| | EYHADGGKV, IWIPDGENV, | VWSYGVTVW-AL-EYLVPQQGF-ADLK-DVWSYGVTV- |
| | YGVTVWELM, QLFEDNYAL, | PDLK-RFRELVSEF-PDLK-LSYMPIWKF-ADL- |
| | RFRELVSEF, EYVNARHCL, | SYGVTVWEL-ADA-QCVNCSQFL-ADAK-VYMIMVKCW- |
| | KWMALESIL, DLLEKGERL, | AILK-KWMALESIL-AI-MIMVKCWMI |
| | LSYMPIWKF, LRIVRGTQL, | (SEQ ID NO: 194) |
| | VWSYGVTVW, ILLVVVLGV, | |
| | QCVNCSQFL, LAALCRWGL | |
| | (SEQ ID NOS: . . . 184, 30, | |
| | 54, 45, 185, 115, 186, | |
| | 129, 187, 153, 98, 27, | |
| | 188, 189, 190, 191, | |
| | 62, 39, 192, 66, 193, | |
| | and 22, respectively) | |
| A*2402 | VYMIMVKCW, LVVVLGVVF, | AWPDSLPDL-DLLEKGERL-RDG-PYVSRLLGI-PDL- |
| | TLQGLGISW, SYGVTVWEL, | TLQGLGISW-A-SLAFLPESF-PDGK-AVVGILLVV-RT- |
| | EYLVPQQGF, SLAFLPESF, | LVVVLGVVF-A-IWIPDGENV-RLL-VWSYGVTVW-AL- |
| | TYLPTNASL, PYVSRLLGI, | EYLVPQQGF-ADLK-QLMPYGCLL-AD-SYGVTVWEL-ADL- |
| | RIVRGTQLF, IWIPDGENV, | TYLPTNASL-A-RIVRGTQLF-RWGLLLALL-A- |
| | RWGLLLALL, KWMALESIL, | KWMALESIL-AIGV-VYMIMVKCW |
| | DLLEKGERL, QLMPYGCLL, | (SEQ ID NO: 197) |
| | VWSYGVTVW, AVVGILLVV, | |
| | AWPDSLPDL | |
| | (SEQ ID NOS: 184, 25, | |
| | 68, 185, 186, 195, | |
| | 129, 144, 75, 153, 54, | |
| | 190, 191, 43, 192, | |
| | 120, and 196 . . . , | |
| | respectively) | |
| A*2403 | VYMIMVKCW, FYRSLLEDD, | RMARDPQRF-AD-AVRGTQLFED-RD-LQPEQLQVF-ADG- |
| | CYGLGMEHL, LQGLGISWL, | EYVNARHCL-ADA-RWGLLLALL-ASEGAGSDVF- |
| | SEGAGSDVF, SYGVTVWEL, | AGEGLACHQL-PDLK-LQGLGISWL-AI-SYGVTVWEL-AD- |
| | EYLVPQQGF, LQPEQLQVF, | AWPDSLPDL-PL-EYLVPQQGF-ADGK-HNGAYSLTL- |
| | TYLPTNASL, RMARDPQRF, | AFNHSGICEL-A-YLVPQQGFF-ADGV-AYSLTLQGL- |
| | VTVWELMTF, YLVPQQGFF, | PDLK-RFRELVSEF-ADGK-ACYGLGMEHL-AL- |
| | RWGLLLALL, HNGAYSLTL, | VWSYGVTVW-AI-AFQNLQVIRG-ADG-VTVWELMTF- |
| | GEGLACHQL, RFRELVSEF, | ADGK-AFYRSLLEDD-RDL-TYLPTNASL-AI- |
| | EYVNARHCL, KWMALESIL, | VYMIMVKCW-AILK-KWMALESIL-AD-RFTHQSDVW |
| | FQNLQVIRG, AYSLTLQGL, | (SEQ ID NO: 211) |
| | VWSYGVTVW, RFTHQSDVW, | |
| | FNHSGICEL, VRGTQLFED, | |
| | AWPDSLPDL | |
| | (SEQ ID NOS: 184, 198, | |
| | 199, 200, 201, 185, | |
| | 186, 41, 129, 202, | |
| | 203, 204, 54, 205, | |
| | 206, 188, 189, 190, | |
| | 207, 134, 192, 208, | |
| | 209, 210, and 196 . . . , | |
| | respectively) | |
| A*2601 | SLRELGSGL, ISWLGLRSL, | CTIDVYMIM-PI-ICELHCPAL-A-QLVTQLMPY-ADG- |
| | CRWGLLLAL, ETLEEITGY, | VSRLLGICL-ALCRWGLLL-PDLK-ARDEAYVMAGV-AD- |
| | LQPEQLQVF, VTVWELMTF, | ETLEEITGY-A-TEILKGGVL-P-QLFEDNYAL-PD- |
| | DTILWKDIF, HTVPWDQLF, | LQPEQLQVF-AD-KVPIKWMAL-SIISAVVGI-RD- |
| | ICELHCPAL, VSRLLGICL, | DTILWKDIF-ALGV-AETHLDMLRH-A-DVFDGDLGM- |
| | ALCRWGLLL, QLFEDNYAL, | PDLK-SLRELGSGL-STVQLVTQL-PLGK-ISWLGLRSL-- |
| | DVFDGDLGM, ETHLDMLRH, | AFDGDLGMGA-AD-CRWGLLLAL-PD-VTVWELMTF-ADGK- |
| | FDGDLGMGA, KVPIKWMAL, | AFEDNYALAV-RDLK-HTVPWDQLF |
| | DEAYVMAGV, CTIDVYMIM, | (SEQ ID NO: 224) |
| | TEILKGGVL, QLVTQLMPY, | |
| | STVQLVTQL, SIISAVVGI, | |
| | FEDNYALAV | |
| | (SEQ ID NOS: 141, 212, | |
| | 21, 213, 41, 203, 214, | |
| | 215, 216, 217, 74, 27, | |
| | 218, 219, 32, 220, 50, | |
| | 221, 222, 163, 223, | |
| | 37, and 55 . . . , | |
| | respectively) | |
| A*2902 | DVWSYGVTV, LTCSPQPEY, | LHCPALVTY-SD-LTCSPQPEY-ADL-RLVHRDLAA-ALG- |
| | GSGAFGTVY, ICLTSTVQL, | HLDMLRHLY-AD-LVVVLGVVF-PDGK-DIFHKNNQL-AD- |
| | VMAGVGSPY, DIFHKNNQL, | LEEITGYLY-AD-GVVKDVFAF-AD-ARPGGLRELQL-AD- |
| | YLEDVRLVH, THQSDVWSY, | ETLEEITGY-ALL-THQSDVWSY-AD-AYLEDVRLVH- |
| | GTVYKGIWI, ETLEEITGY, | PDLK-QVVQGNLEL-AI-GSGAFGTVY-RL-VMAGVGSPY- |
| | GVVKDVFAF, SMPNPEGRY, | AILK-LMTFGAKPY-AD-GTQLFEDNY-ADGK- |
| | GTQLFEDNY, VCTGTDMKL, | CVTACPYNY-ADG-GTVYKGIWI-ADL-SMPNPEGRY- |
| | MQIAKGMSY, HTVPWDQLF, | ADLK-HTVPWDQLF-ADLK-SLTLQGLGI-AD- |
| | LEEITGYLY, RLVHRDLAA, | MQIAKGMSY-A-ICLTSTVQL-SD-DVWSYGVTV-PDLK- |
| | LVVVLGVVF, LMTFGAKPY, | MSYLEDVRL-RD-VCTGTDMKL-AD-FSPAFDNLY-AIL- |
| | HLDMLRHLY, QVVQGNLEL, | SPAFDNLYY |
| | SLTLQGLGI, SPAFDNLYY, | (SEQ ID NO: 239) |
| | FSPAFDNLY, PGGLRELQL, | |
| | LHCPALVTY, CVTACPYNY, | |
| | MSYLEDVRL | |
| | (SEQ ID NOS: 30, 94, | |
| | 58, 225, 159, 45, 226, | |
| | 227, 228, 213, 63, | |
| | 229, 230, 231, 61, | |
| | 215, 232, 117, 25, | |
| | 233, 101, 234, 122, | |
| | 235, 105, 236, 38, | |
| | 237, and 238 . . . , | |
| | respectively) | |
| A*3001 | HYKDPPFCV, GGKVPIKWM, | KIRKYTMRR-A-YLYISAWPD--LVKSPNHVK-PLLK- |
| | RSRACHPCS, HVRENRGRL, | KVKVLGSGA-PDG-KETELRKVK-PD-AIKVLRENT-AD- |
| | MARDPQRFV, AARNVLVKS, | GGKVPIKWM-ADG-NVKIPVAIK-AD-ARGGCLLDHVRE- |
| | LVKSPNHVK, TQRCEKCSK, | AGLRSLRELG-ADG-RPKTLSPGK-AI-LQRLRIVRG- |
| | LQRLRIVRG, STFKGTPTA, | PDGV-KLRLPASPE-A-WGLLLALLP-AD-RSRACHPCS- |
| | KLRLPASPE, YLYISAWPD, | AILK-KRRQQKIRK-ADLK-HVRENRGRL-AD- |
| | KRRQQKIRK, KETELRKVK, | ARPGKNGVVKD-A-PLQRLRIVR-RDAK-AARNVLVKS-AD- |
| | NVKIPVAIK, RPKTLSPGK, | MARDPQRFV-A-VLRENTSPK-ADL-VARCPSGVK-ADL- |
| | KIFGSLAFL, VARCPSGVK, | HYKDPPFCV-AD-KIFGSLAFL-A-STFKGTPTA-ADL- |
| | AIKVLRENT, GCLLDHVRE, | TQRCEKCSK |
| | WGLLLALLP, KIRKYTMRR, | (SEQ ID NO: 258) |
| | PLQRLRIVR, PGKNGVVKD, | |
| | VLRENTSPK, GLRSLRELG, | |
| | KVKVLGSGA | |
| | (SEQ ID NOS: 139, 240, | |
| | 241, 242, 243, 244, | |
| | 166, 245, 246, 247, | |
| | 248, 169, 249, 250, | |
| | 251, 252, 31, 253, 64, | |
| | 254, 24, 180, 48, 255, | |
| | 174, 256, and 257 . . . , | |
| | respectively) | |
| A*3002 | ESFDGDPAS, KGMSYLEDV, | SMPNPEGRY-ADL-KHSDCLACL--ADMGDLVDAE-RDGK- |
| | LTCSPQPEY, VMAGVGSPY, | CVTACPYNY-AL-GGAVENPEY-AL-AVVKDVFAFG-PLAK- |
| | VVKDVFAFG, DMGDLVDAE, | AEIPDLLEKG-PDGK-HLDMLRHLY-ADLK-TVWELMTFG- |
| | THQSDVWSY, GGAVENPEY, | AD-LTCSPQPEY-ADL-RSSSTRSGG-ADGK-ETLEEITGY- |
| | SLTEILKGG, ETLEEITGY, | AD-VLQGLPREY-AD-ARPLTSIISAV-AL-ASCVTACPY- |
| | SMPNPEGRY, GTQLFEDNY, | PLL-SAVVGILLV-ADLV-AESFDGDPAS-R-DVFDGDLGM- |
| | SLPDLSVFQ, RSSSTRSGG, | PIL-AAPRSPLAPS-AI-GTQLFEDNY-AIG- |
| | HLDMLRHLY, LMTFGAKPY, | ASLTEILKGG-AD-KGMSYLEDV-AD-VMAGVGSPY-ATLK- |
| | VLQGLPREY, PLTSIISAV, | SLPDLSVFQ-RDLK-THQSDVWSY-ADA-SPAFDNLYY- |
| | ASCVTACPY, DVFDGDLGM, | ADL-FSPAFDNLY-ADLK-YYWDQDPPE-ADLV- |
| | SAVVGILLV, TVWELMTFG, | LMTFGAKPY |
| | SPAFDNLYY, FSPAFDNLY, | (SEQ ID NO: 270) |
| | YYWDQDPPE, EIPDLLEKG, | |
| | APRSPLAPS, KHSDCLACL, | |
| | CVTACPYNY | |
| | (SEQ ID NOS: 259, 260, | |
| | 94, 159, 261, 262, | |
| | 227, 263, 264, 213, | |
| | 229, 230, 130, 265, | |
| | 101, 233, 266, 143, | |
| | 46, 218, 155, 168, | |
| | 235, 105, 267, 268, | |
| | 269, 128, and 237 . . . , | |
| | respectively) | |
| A*3101 | VVFGILIKR, KVPIKWMAL, | QALLHTANR-AIG-RQVPLQRLR-ADGK-QKIRKYTMR- |
| | GMEHLREVR, QKIRKYTMR, | ADGK-GVGSPYVSR-RILKETELR-ADL-LEDVRLVHR- |
| | TVCAGGCAR, MALESILRR, | ADG-TLIDTNRSR-ADL-GMEHLREVR-ADGK- |
| | SPLDSTFYR, GVGSPYVSR, | REGPLPAAR-RIG-MALESILRR-PDGK-LGISWLGLR- |
| | KITDFGLAR, RILKETELR, | ADGV-KITDFGLAR-A-PLQRLRIVR-ADG-VVFGILIKR- |
| | LVHRDLAAR, LACHQLCAR, | RDGK-LVHRDLAAR-A-TVCAGGCAR-RDG-KIRKYTMRR- |
| | PLQRLRIVR, VSEFSRMAR, | ADG-AALCRWGLL-ADGK-KIFGSLAFL-PDG- |
| | LEDVRLVHR, VFQNLQVIR, | KVPIKWMAL-SD-ASPLDSTFYR-ADL-VSEFSRMAR- |
| | LGISWLGLR, AALCRWGLL, | ADLV-CVNCSQFLR-ADLK-LACHQLCAR-AD- |
| | QALLHTANR, KIFGSLAFL, | VFQNLQVIR-AIL-SWLGLRSLR |
| | CVNCSQFLR, REGPLPAAR, | (SEQ ID NO: 285) |
| | KIRKYTMRR, TLIDTNRSR, | |
| | RQVPLQRLR, SWLGLRSLR | |
| | (SEQ ID NOS: . . . 158, | |
| | 220, 73, 271 52, 272, | |
| | 273, 274, 26, 275, 33, | |
| | 276, 48, 277, 278, | |
| | 279, 59, 280, 281, 31, | |
| | 175, 282, 180, 283, | |
| | 284, and 65, | |
| | respectively) | |
| B*0702 | RCEKCSKPC, SPKANKEIL, | Var1: AAPRSPLAPS-ALPAARPAGA-PDG- |
| | SPETHLDML, PPSPREGPL, | ALPTHDPSPL-A-ALPASPETHL-SD-ASPETHLDML- |
| | GAVENPEYL, GVVKDVFAF, | AVLDNGDPL--ASPKANKEIL-P-GAVENPEYL-- |
| | SPGKNGVVK, AVLDNGDPL, | ASPGKNGVVK-AD-LPTNASLSF-ADPASNTAPL-- |
| | HVRENRGRL, AARPAGATL, | AARPAGATL-AAPQPHPPPA-ADGV-LQVIRGRIL-PDG- |
| | MPNQAQMRI, LPTHDPSPL, | RASPLTSII-ADL-APPSPREGPL-RDLK-HVRENRGRL- |
| | RASPLTSII, RKYTMRRLL, | SDL-AHPPPAFSPA-PDLK-AMPNQAQMRI-ADLV- |
| | SPREGPLPA, GSCTLVCPL, | RKYTMRRLL-A-GVVKDVFAF-AD-AVPLQRLRIV-ADGK- |
| | DPASNTAPL, LPAARPAGA, | GSCTLVCPL-AI-ASPREGPLPA-ADL-RCEKCSKPC |
| | APQPHPPPA, HPPPAFSPA, | (SEQ ID NO: 304) |
| | LPTNASLSF, VPLQRLRIV, | Var2: |
| | LPASPETHL, APRSPLAPS, | MELAALCRWGLLLALLPPGAPASPKANKEILAARPAGATLAL |
| | LQVIRGRIL | PTHDPSPLAALPASPETHLSDASPETHLDMLADAPPSPREGP |
| | (SEQ ID NOS: 286, 287, | LRDLKHVRENRGRLADLACPSGVKPDLADGSTRSGGGDLPIA |
| | 288, 289, 290, 63, | SPLTSIISA |
| | 291, 152, 242, 292, | (SEQ ID NO: 305) |
| | 293, 294, 35, 295, | |
| | 296, 76, 297, 298, | |
| | 299, 300, 301, 302, | |
| | 303, 269, and 49, | |
| | respectively) | |
| B*0801 | LVVVLGVVF, VVGILLVVV, | Var1: YISAWPDSL-PDL-ECRPRFREL-AD- |
| | ILRRRFTHQ, VLIQRNPQL, | VGILLVVVL-PD-QQKIRKYTM-AD-LFRNPHQAL-AL- |
| | QQKIRKYTM, ISAVVGILL, | LIKRRQQKI-ADLK-AYGVTVWELM-PDLK-LGMEHLREV- |
| | SPKANKEIL, DIFHKNNQL, | ASPKANKEIL-ALIHHNTHL-A-DIFHKNNQL-AD- |
| | FGLARLLDI, MIMVKCWMI, | MVHHRHRSS-AD-AVPLQRLRIV-A-ILLVVVLGV-AD- |
| | YGVTVWELM, SLAFLPESF, | VSRLLGICL-AFGLARLLDI-AI-LQRLRIVRG-AD- |
| | LAALCRWGL, LQRLRIVRG, | VVGILLVVV-PDG-KVPIKWMAL-SLAFLPESF-AI- |
| | LGMEHLREV, MVHHRHRSS, | LQVIRGRIL-LVVVLGVVF-A-MRILKETEL-RTG- |
| | LDSTFYRSL, ALIHHNTHL, | VLIQRNPQL-PDLK-ILRRRFTHQ-AD-LAALCRWGL-AD- |
| | MRILKETEL, VSRLLGICL, | LDSTFYRSL-RD-LRIVRGTQL-PIAK-ISAVVGILL-AI- |
| | ECRPRFREL, LFRNPHQAL, | MIMVKCWMI |
| | KVPIKWMAL, LRIVRGTQL, | (SEQ ID NO: 319) |
| | VPLQRLRIV, YISAWPDSL, | Var2: |
| | LIKRRQQKI, LQVIRGRIL, | MELAALCRWGLLLALLPPGAPAIGFHKNNQLALASPKANKEI |
| | VGILLVVVL, ILLVVVLGV | LRDGKDIFHKNNQLPDGKLGMEHLREVADLFRNPHQALALLG |
| | (SEQ ID NOS: . . . 25, 306, | CKKIFGSLPDLRIVRGTQLADGVMRILKETELSDGQLRSLTE |
| | 307, 308, 309, 60, | ILADGKECRPRFRELADGQLMPYGCLLPDLK |
| | 287, 45, 310, 115, 98, | (SEQ ID NO: 320) |
| | 195, 22, 246, 311, | |
| | 312, 313, 119, 314, | |
| | 217, 315, 316, 220, | |
| | 39, 302, 28, 317, 49, | |
| | 318, and 66, | |
| | respectively) | |
| B*1501 | WCMQIAKGM, LTCSPQPEY, | LVVVLGVVF-A-IQRNPQLCY-AILV-TQCVNCSQF-ADG- |
| | GSGAFGTVY, VMAGVGSPY, | TLIDTNRSR-ASEGAGSDVF-ALIHHNTHL-AI- |
| | IQRNPQLCY, ISWLGLRSL, | AYGVTVWELM-AIGK-ISWLGLRSL-S-VKVLGSGAF-A- |
| | SEGAGSDVF, YGVTVWELM, | QLFEDNYAL-PLG-RELGSGLAL-ASCVTACPY-AIL- |
| | TQCVNCSQF, SLAFLPESF, | VTSANIQEF-AIG-VQGNLELTY-AD-LTCSPQPEY-ADLK- |
| | LQVIRGRIL, VTSANIQEF, | QVVQGNLEL-AI-GSGAFGTVY-RL-VMAGVGSPY-ADGV- |
| | VQGNLELTY, RIVRGTQLF, | LQVIRGRIL-SLAFLPESF-ADG-VWSYGVTVW-ADA- |
| | MQIAKGMSY, ALIHHNTHL, | RIVRGTQLF-WCMQIAKGM-AD-MQIAKGMSY-A- |
| | LVVVLGVVF, LMTFGAKPY, | LMTFGAKPY-RDL-RACHPCSPM |
| | RELGSGLAL, ASCVTACPY, | (SEQ ID NO: 327) |
| | VKVLGSGAF, RACHPCSPM, | |
| | QVVQGNLEL, VWSYGVTVW, | |
| | QLFEDNYAL, TLIDTNRSR | |
| | (SEQ ID NOS: . . . 321, 94, | |
| | 58, 159, 322, 212, | |
| | 201, 98, 323, 195, 49, | |
| | 324, 325, 75, 61, 119, | |
| | 25, 233, 23, 46, 40, | |
| | 326, 234, 192, 27, and | |
| | 283, respectively) | |
| B*1801 | DVWSYGVTV, SEGAGSDVF, | LRIVRGTQL-ASEGAGSDVF-ALDIDETEYH-ADLK- |
| | LELTYLPTN, SAWPDSLPD, | QETELVEPL-AD-ARPEYLTPQGG-ADGV-EEITGYLYI- |
| | TELVEPLTP, QETELVEPL, | PDGK-EECRVLQGL-ADG-RELGSGLAL-AEDLGPASPL-A- |
| | EECRVLQGL, MQIAKGMSY, | TEILKGGVL-P-LEEITGYLY-PLGK-AGDLGMGAAK-AD- |
| | LEEITGYLY, LDIDETEYH, | LELTYLPTN-RDG-VKVLGSGAF-AD-TELVEPLTP-RDLK- |
| | PEYLTPQGG, QRFVVIQNE, | SAWPDSLPD-AD-DVWSYGVTV-AD-MQIAKGMSY-AD- |
| | GDLGMGAAK, RELGSGLAL, | QRFVVIQNE |
| | LRIVRGTQL, VKVLGSGAF, | (SEQ ID NO: 335) |
| | EEITGYLYI, TEILKGGVL, | |
| | EDLGPASPL | |
| | (SEQ ID NOS: 30, 201, | |
| | 165, 328, 47, 44, 51, | |
| | 61, 232, 329, 330, | |
| | 331, 332, 23, 39, 40, | |
| | 333, 222, and 334, | |
| | respectively) | |
| B*2705 | GRILHNGAY, RRLLQETEL, | GRILHNGAY-ADG-CRWGLLLAL-LQPEQLQVF- |
| | ILDEAYVMA, ARPAGATLE, | AILDEAYVMA-RD-AKGLQSLPT-AD-GRLGSQDLL-ADG- |
| | GRLGSQDLL, YLEDVRLVH, | RELGSGLAL-AYLEDVRLVH-RD-AFAGCKKIFG-ADG- |
| | CRWGLLLAL, RRFTHQSDV, | FRNPHQALL-PIGK-AGEGLACHQL-AD-ARPAGATLE-SL- |
| | HRDLAARNV, FAGCKKIFG, | RRLLQETEL-AAGCTGPKH-AD-AVRGTQLFED-RDLV- |
| | RRQQKIRKY, AAGCTGPKH, | RKYTMRRLL-RD-LRIVRGTQL-PDLK-RNPQLCYQD- |
| | FRNPHQALL, ARVCYGLGM, | ADLK-RQVPLQRLR-ADAK-ARVCYGLGM-ADGV- |
| | RKYTMRRLL, QRFVVIQNE, | HRDLAARNV-PD-QRASPLTSI-PLLK-HRHRSSSTR- |
| | QRASPLTSI, YTMRRLLQE, | ADLV-YLYISAWPD-ADAK-QRFVVIQNE-ADLV- |
| | CRVLQGLPR, AKGLQSLPT, | RRQQKIRKY-ADLK-CRVLQGLPR-ADL-YTMRRLLQE- |
| | LQPEQLQVF, VRGTQLFED, | ADLK-RRFTHQSDV |
| | RNPQLCYQD, YLYISAWPD, | (SEQ ID NO: 351) |
| | LRIVRGTQL, GEGLACHQL, | |
| | HRHRSSSTR, RELGSGLAL, | |
| | RQVPLQRLR | |
| | (SEQ ID NOS: 336, 337, | |
| | 112, 338, 339, 226, | |
| | 21, 340, 341, 342, | |
| | 343, 344, 131, 345, | |
| | 295, 331, 346, 171, | |
| | 347, 348, 41, 210, | |
| | 349, 169, 39, 206, | |
| | 350, 23, and 284, | |
| | respectively) | |
| B*3501 | LTCSPQPEY, LALLPPGAA, | Var1: |
| | EPLTPSGAM, DPASNTAPL, | HTVPWDQLF-ADLV-CRWGLLLAL-RI- |
| | CRWGLLLAL, MPYGCLLDH, | ALDIDETEYH-ADL-ARDGDLGMGAA-RD-LPTNASLSF- |
| | DGDLGMGAA, GVVKDVFAF, | ADPASNTAPL-ALPTHDPSPL-AD-NKEILDEAY-- |
| | MSYLEDVRL, EILDEAYVM, | ADPAPGAGGM-AI-AEPLTPSGAM-A-GVVKDVFAF-AD- |
| | LVTYNTDTF, MPNQAQMRI, | LTCSPQPEY-ADLK-LVTYNTDTF-AD-LALLPPGAA-PD- |
| | LPTHDPSPL, TPTAENPEY, | EILDEAYVM-P-LVVVLGVVF-AECVGEGLAC-A- |
| | ICELHCPAL, DPAPGAGGM, | TPTAENPEY-AD-RSLLEDDDM-ALLV-FVVIQNEDL-AL- |
| | LVVVLGVVF, FSPAFDNLY, | AMPNQAQMRI-ADLV-MSYLEDVRL-AI-LMTFGAKPY-AD- |
| | LMTFGAKPY, HTVPWDQLF, | ICELHCPAL-ALGK-YYWDQDPPE-ADL-SPAFDNLYY- |
| | FVVIQNEDL, RSLLEDDDM, | ADL-FSPAFDNLY-AILK-AMPYGCLLDH |
| | SPAFDNLYY, NKEILDEAY, | (SEQ ID NO: 363) |
| | LPTNASLSF, ECVGEGLAC, | Var2: |
| | YYWDQDPPE, LDIDETEYH | MELAALCRWGLLLALLPPGAPADGKTPTAENPEYAALPASPE |
| | (SEQ ID NOS: 94, 352, | THLPILKYSEDPTVPLPDGALPTHDPSPLADNKEILDEAYAD |
| | 353, 297, 21, 354, | EILDEAYVMPLVVVLGVVFADMQIAKGMSYALMTFGAKPYPL |
| | 104, 63, 238, 355, | GKAPPPAFSPAFADLHCPALVTY |
| | 356, 293, 294, 357, | (SEQ ID NO: 364) |
| | 216, 358, 25, 105, | |
| | 233, 215, 359, 360, | |
| | 235, 361, 301, 362, | |
| | 267, and 329, | |
| | respectively) | |
| B*4001 | REVRAVTSA, SETDGYVAP, | MELAALCRW-RDLAARNVL-PDA-QETELVEPL- |
| | SEGAGSDVF, QVVQGNLEL, | AEEEAPRSPL-PDGK-EECRVLQGL-ADA-GERLPQPPI- |
| | AENPEYLGL, RDLAARNVL, | ADG-SETDGYVAP-PDA-AGEGLACHQL-ADG- |
| | LEDDDMGDL, QETELVEPL, | RELGSGLAL-P-QLFEDNYAL-PD-ALEDDDMGDL-PDLK- |
| | EEEAPRSPL, RELGSGLAL, | REVRAVTSA-ASEGAGSDVF-A-TEILKGGVL-PL- |
| | ALCRWGLLL, MELAALCRW, | EEITGYLYI-PDGK-AENPEYLGL-PDLK-QEVQGYVLI- |
| | EECRVLQGL, QLFEDNYAL, | AD-EQLQVFETL-A-QVVQGNLEL-A-QEFAGCKKI-- |
| | QEVQGYVLI, QEFAGCKKI, | ALCRWGLLL-RD-AFEDNYALAV |
| | EQLQVFETL, EEITGYLYI, | (SEQ ID NO: 374) |
| | GEGLACHQL, TEILKGGVL, | |
| | GERLPQPPI, FEDNYALAV | |
| | (SEQ ID NOS: 365, 366, | |
| | 201, 234, 367, 368, | |
| | 369, 44, 370, 23, 74, | |
| | 34, 51, 27, 56, 371, | |
| | 372, 333, 206, 222, | |
| | 373, and 55, | |
| | respectively) | |
| B*4002 | HYKDPPFCV, CQSLTRTVC, | ISWLGLRSL-AEEEAPRSPL-RDLAARNVL-RLG- |
| | RELQLRSLT, REVRAVTSA, | GENVKIPVA-RLG-KHSDCLACL-AIG-GERLPQPPI-ADL- |
| | TFYRSLLED, ISWLGLRSL, | TGTDMKLRL-PDGK-AENPEYLGL-ADG-RELGSGLAL-- |
| | TLQGLGISW, KHSDCLACL, | REVRAVTSA-ADG-REYVNARHC-A-QEFAGCKKI-A- |
| | AENPEYLGL, CRWGLLLAL, | QETELVEPL-A-TELRKVKVL-TDMKLRLPA-ADLK- |
| | TRTVCAGGC, TDMKLRLPA, | QEVQGYVLI-PDL-ARGGSRCWGESS-ALGV-KITDFGLAR- |
| | RDLAARNVL, QETELVEPL, | A-TDFGLARLL-PDA-RKYTMRRLL-ADG-RELQLRSLT- |
| | EEEAPRSPL, RKYTMRRLL, | ADLK-LDSTFYRSL-MELAALCRW-A-TLQGLGISW-ADL- |
| | MELAALCRW, RELGSGLAL, | CQSLTRTVC-ALL-HYKDPPFCV-AIG-YISAWPDSL-AD- |
| | QEFAGCKKI, TDFGLARLL, | CRWGLLLAL-RDL-TRTVCAGGC-ADLK-TFYRSLLED |
| | GENVKIPVA, LDSTFYRSL, | (SEQ ID NO: 384) |
| | QEVQGYVLI, KITDFGLAR, | |
| | TELRKVKVL, GSRCWGESS, | |
| | TGTDMKLRL, REYVNARHC, | |
| | GERLPQPPI, YISAWPDSL | |
| | (SEQ ID NOS: 139, 375, | |
| | 376, 365, 161, 212, | |
| | 68, 128, 367, 21, 377, | |
| | 378, 368, 44, 370, | |
| | 295, 34, 23, 371, 69, | |
| | 379, 313, 56, 26, 380, | |
| | 381, 382, 383, 373, | |
| | and 28, respectively) | |
| B*4402 | TRTVCAGGC, TLQGLGISW, | TRTVCAGGC-ADG-GGGDLTLGL-ARPEADQCVAC-A- |
| | VKVLGSGAF, QETELVEPL, | TLQGLGISW-AI-AFDGDLGMGA-PDAK-ARGDLTLGLEP- |
| | ERGAPPSTF, IDSECRPRF, | PDGK-IDSECRPRF-ADG-VKVLGSGAF-ADG- |
| | RELGSGLAL, MELAALCRW, | QETELVEPL-ADG-RELGSGLAL-A-QEVQGYVLI-ALG- |
| | FDGDLGMGA, GGGDLTLGL, | ERGAPPSTF-A-QEFAGCKKI-MELAALCRW-ALG- |
| | QEFAGCKKI, QEVQGYVLI, | VKIPVAIKV-AL-LHCPALVTY |
| | LHCPALVTY, PEADQCVAC, | (SEQ ID NO: 389) |
| | VKIPVAIKV, GDLTLGLEP | |
| | (SEQ ID NOS: . . . 377, | |
| | 68, 40, 44, 385, 386, | |
| | 23, 34, 32, 387, 371, | |
| | 56, 38, 388, 42, and | |
| | 29, respectively) | |
| B*4403 | FDGDLGMGA, TRTVCAGGC, | LRIVRGTQL-PIAA-GGGDLTLGL-ARPEADQCVAC-AI- |
| | SEGAGSDVF, VKVLGSGAF, | AFDGDLGMGA-PDAK-ARGDLTLGLEP-PDLK- |
| | PDLSVFQNL, QETELVEPL, | QETELVEPL-PI-VKVLGSGAF-ASEGAGSDVF-PDG- |
| | EECRVLQGL, LEEITGYLY, | RELGSGLAL-A-QEVQGYVLI-ADGK-EECRVLQGL-PDLK- |
| | LRIVRGTQL, RELGSGLAL, | LEEITGYLY-A-TEILKGGVL-PL-EEITGYLYI-AD- |
| | MELAALCRW, GDLTLGLEP, | MELAALCRW-AD-ARPDLSVFQNL-ADL-TDFGLARLL-PD- |
| | GGGDLTLGL, TDFGLARLL, | TRTVCAGGC |
| | EEITGYLYI, QEVQGYVLI, | (SEQ ID NO: 391) |
| | TEILKGGVL, PEADQCVAC | |
| | (SEQ ID NOS: 32, 377, | |
| | 201, 40, 390, 44, 51, | |
| | 232, 39, 23, 34, 29, | |
| | 387, 69, 333, 56, 222, | |
| | and 388, respectively) | |
| B*4501 | PEGRYTFGA, RELQLRSLT, | CELHCPALV-ADG-GENVKIPVA-ALPASPETHL-RD- |
| | MEHLREVRA, FDGDLGMGA, | ARPEGRYTFGA-ADGK-IDSECRPRF-ADLK-GERLPQPPI- |
| | REVRAVTSA, VSRLLGICL, | AIL-AEEAPRSPLA-ADGA-EEITGYLYI-ALPAARPAGA- |
| | GERLPQPPI, LGMGAAKGL, | PDGK-MEHLREVRA-PDG-RELQLRSLT-ADLK- |
| | LPAARPAGA, TSANIQEFA, | KEILDEAYV-AT-AFDGDLGMGA-PDLK-REVRAVTSA-- |
| | LDSTFYRSL, IDSECRPRF, | ALPSETDGYV-ADG-AEQRASPLT-ADG-AGEGLACHQL- |
| | LPSETDGYV, RELGSGLAL, | ADG-RELGSGLAL-AD-CEKCSKPCA-ADGV-QEVQGYVLI- |
| | ASCVTACPY, QEVQGYVLI, | ADL-TSANIQEFA-AD-LDSTFYRSL-MELAALCRW-ATGK- |
| | AEQRASPLT, MELAALCRW, | AINCTHSCVD-RD-AFEDNYALAV-RD-LGMGAAKGL-- |
| | GENVKIPVA, INCTHSCVD, | VSRLLGICL-PD-VKIPVAIKV-AI-ASCVTACPY |
| | EEITGYLYI, GEGLACHQL, | (SEQ ID NO: 403) |
| | LPASPETHL, FEDNYALAV, | |
| | EEAPRSPLA, CELHCPALV, | |
| | KEILDEAYV, VKIPVAIKV, | |
| | CEKCSKPCA | |
| | (SEQ ID NOS: 392, 376, | |
| | 393, 32, 365, 217, | |
| | 373, 394, 298, 395, | |
| | 313, 386, 396, 23, 46, | |
| | 56, 397, 34, 379, 398, | |
| | 333, 206, 303, 55, | |
| | 399, 400, 401, 42, and | |
| | 402, respectively) | |
| B*5101 | LQLRSLTEI, LPQPPICTI, | CRWGLLLAL-PD-ENVKIPVAI-AYGVTVWELM-A- |
| | KGMSYLEDV, CRWGLLLAL, | ALPASPETHL-ARPDLSVFQNL-PD-LPTNASLSF-ADG- |
| | PDLSVFQNL, YGVTVWELM, | ALPTHDPSPL-PDL-ALPSETDGYV-PDLK-LGMEHLREV- |
| | LGMEHLREV, LGMGAAKGL, | AD-LPQPPICTI-ADGV-QEVQGYVLI-AD-EQLQVFETL- |
| | MPNQAQMRI, LPTHDPSPL, | A-LGMGAAKGL-PD-KGMSYLEDV-A-QEFAGCKKI-S- |
| | ENVKIPVAI, QEFAGCKKI, | VGILLVVVL--AMPNQAQMRI-ADLK-LQLRSLTEI-AD- |
| | TDFGLARLL, EQLQVFETL, | VKIPVAIKV-A-TDFGLARLL |
| | LPTNASLSF, QEVQGYVLI, | (SEQ ID NO: 406) |
| | LPASPETHL, LPSETDGYV, | |
| | VKIPVAIKV, VGILLVVVL | |
| | (SEQ ID NOS: . . . 150, | |
| | 404, 260, 21, 390, 98, | |
| | 311, 394, 293, 294, | |
| | 405, 371, 69, 372, | |
| | 301, 56, 303, 396, 42, | |
| | and 318, respectively) | |
| B*5301 | DDMGDLVDA, LPQPPICTI, | ASPLDSTFYR-ADG-VENPEYLTP-A-ALPASPETHL- |
| | CRWGLLLAL, SPLDSTFYR, | ARAGVGSPYVS-RD-LPTNASLSF-ADG-ALPTHDPSPL- |
| | RPEDECVGE, MPNQAQMRI, | ADL-LERPKTLSP-AL-AFDGDLGMGA-PDAK- |
| | LPTHDPSPL, TPTAENPEY, | ARGDLTLGLEP-PDL-ARDDMGDLVDA-PDL- |
| | SPQPEYVNQ, VENPEYLTP, | ARPEDECVGE-A-TPTAENPEY-AL-AMPNQAQMRI-ADLK- |
| | AGVGSPYVS, SPLTSIISA, | LPQPPICTI-AD-ASPLTSIISA-AD-CRWGLLLAL- |
| | FDGDLGMGA, SPAFDNLYY, | AGPLPAARPA-PD-AAPRSPLAPS-ALA-ASPQPEYVNQ- |
| | LERPKTLSP, LPTNASLSF, | ALG-VKIPVAIKV-AD-ACPSGVKPDL-AD-LHCPALVTY- |
| | GDLTLGLEP, LPASPETHL, | SDA-SPAFDNLYY |
| | LHCPALVTY, APRSPLAPS, | (SEQ ID NO: 415) |
| | GPLPAARPA, VKIPVAIKV, | |
| | CPSGVKPDL | |
| | (SEQ ID NOS: 407, 404, | |
| | 21, 273, 408, 293, | |
| | 294, 357, 409, 410, | |
| | 411, 412, 32, 235, | |
| | 177, 301, 29, 303, 38, | |
| | 269, 413, 42, and 414, | |
| | respectively) | |
| B*5401 | LVEPLTPSG, IWIPDGENV, | AWKDIFHKNN-AD-AFDGDLGMGA-PDLK-REVRAVTSA- |
| | RELQLRSLT, LPQPPICTI, | ALL-AEEAPRSPLA-ADG-ARDGDPASNTA-ALPAARPAGA- |
| | REVRAVTSA, LTSIISAVV, | A-IWIPDGENV-SD-LRENTSPKA-RD-LVEPLTPSG-ADG- |
| | RKVKVLGSG, YKDPPFCVA, | LTSIISAVV-A-RKVKVLGSG-ADGV-RELQLRSLT-ADLK- |
| | SPLAPSEGA, LQRLRIVRG, | LPQPPICTI-AD-LQRLRIVRG-PDLK-RGRILHNGA-AD- |
| | MPYGCLLDH, RGRILHNGA, | ASPLTSIISA-ASPLAPSEGA-ACPALVTYNT-AD- |
| | MPNQAQMRI, CPALVTYNT, | AVPLQRLRIV-ADAA-AMPNQAQMRI-ADLK- |
| | MPIWKFPDE, WKDIFHKNN, | AYKDPPFCVA-RDL-AMPIWKFPDE-ADG-AMPYGCLLDH- |
| | LPAARPAGA, SPLTSIISA, | ADGK-WGLLLALLP |
| | FDGDLGMGA, DGDPASNTA, | (SEQ ID NO: 425) |
| | VPLQRLRIV, WGLLLALLP, | |
| | EEAPRSPLA, LRENTSPKA | |
| | (SEQ ID NOS: 416, 153, | |
| | 376, 404, 365, 417, | |
| | 418, 96, 419, 246, | |
| | 354, 420, 293, 421, | |
| | 422, 423, 298, 412, | |
| | 32, 102, 302, 24, 399, | |
| | and 424, respectively) | |
| B*5701 | DVWSYGVTV, ATLERPKTL, | MELAALCRW-A-VTSANIQEF-ALGK-ENVKIPVAI-ADGK- |
| | GSGAFGTVY, ISWLGLRSL, | DIFHKNNQL-RD-ATLERPKTL-LVVVLGVVF-P- |
| | PAFDNLYYW, DIFHKNNQL, | TLQGLGISW-A-DVFDGDLGM-RDLV-ALCRWGLLL-PDGK- |
| | MSYLEDVRL, VTSANIQEF, | ISWLGLRSL-RSLLEDDDM-ADG-GSGAFGTVY-ADA- |
| | HTVPWDQLF, LVVVLGVVF, | GTQLFEDNY-RDLK-LSYMPIWKF-ADLK-PAFDNLYYW- |
| | TLQGLGISW, ALCRWGLLL, | ADL-QLMPYGCLL-PDLK-MSYLEDVRL-R-DVWSYGVTV- |
| | DVFDGDLGM, MELAALCRW, | PDLK-RFTHQSDVW-ADLV-HTVPWDQLF |
| | RSLLEDDDM, LSYMPIWKF, | (SEQ ID NO: 428) |
| | QLMPYGCLL, ENVKIPVAI, | |
| | RFTHQSDVW, GTQLFEDNY | |
| | (SEQ ID NOS: 30, 426, | |
| | 58, 212, 427, 45, 238, | |
| | 324, 215, 25, 68, 74, | |
| | 218, 34, 360, 62, 43, | |
| | 405, 208, and 230, | |
| | respectively) | |
| B*5801 | RSGGGDLTL, RCEKCSKPC, | PAFDNLYYW-AIL-CTIDVYMIM-ADLV-RMARDPQRF-AD- |
| | VTSANIQEF, TRTVCAGGC, | KGCPAEQRA-PDLK-LGSQDLLNW-AIISAVVGIL-AL- |
| | PAFDNLYYW, ISAVVGILL, | RCEKCSKPC-AIL-VTSANIQEF-ADL-GAMPNQAQM-AD- |
| | RMARDPQRF, VCTGTDMKL, | AVTGASPGGL-P-ISAVVGILL-PD-RSGGGDLTL-- |
| | VTVWELMTF, RIVRGTQLF, | AYLSTDVGSC-A-LAALCRWGL-AL-ASCVTACPY-ADL- |
| | RASPLTSII, HTVPWDQLF, | HTVPWDQLF-ADLK-LSYMPIWKF-ADG-RASPLTSII- |
| | KGCPAEQRA, IISAVVGIL, | ADG-VTVWELMTF-ADGV-ARGQECVEEC-ADL- |
| | LCYQDTILW, ASCVTACPY, | RIVRGTQLF-TRTVCAGGC-AD-KIFGSLAFL-PD- |
| | GAMPNQAQM, CTIDVYMIM, | VCTGTDMKL-AD-LCYQDTILW |
| | VTGASPGGL, YLSTDVGSC, | (SEQ ID NO: 436) |
| | LSYMPIWKF, LGSQDLLNW, | |
| | LAALCRWGL, RGQECVEEC, | |
| | KIFGSLAFL | |
| | (SEQ ID NOS: 429, 286, | |
| | 324, 377, 427, 60, | |
| | 202, 231, 203, 75, 35, | |
| | 215, 430, 71, 431, 46, | |
| | 432, 221, 433, 146, | |
| | 62, 434, 22, 435, and | |
| | 31, respectively) | |

2.3 Th Epitopes

2.3.1 List of Th Epitopes:

2.3.1.1:

(SEQ ID NOS: 437, 39, 438, 439, 440, 441, 442,
443, 444, 445, 446, 447, 448, 449, 450, 451,
452, and 359, respectively)
LRHLYQGCQ,
LRIVRGTQL,
CLHFNHSGICELHCPALV,
LQVFETLEE,
LRSLRELGS,
LCFVHTVPWDQ,
LRGQECVEE,
CPINCTHSC,
IRKYTMRRL,
MRILKETELRKVKVLGS,
VKIPVAIKVLRENTSPK,
YVMAGVGSPYVSRLLGICLTSTVQLV,
VRLVHRDLA,
FGLARLLDIDETEYH,
WMALESILRRRFTHQS,
CTIDVYMIMVKCWMI,
CRPRFRELVSEFS,
FVVIQNEDL

2.3.1.2:

(SEQ ID NOS: _7, 8,9, 10, and 11, respectively)
AVVGILLVVVLGVVFGILIKRRQQKIR,
PICTIDVYMIMVKCWMIDSE,
AQMRILKETELRKVKVLGSGA,
IKWMALESILRRRFTHQSDV,
PICTIDVYMIMVKCWMIDS

2.3.1.3:

(SEQ ID NO: 1)
AKFVAAWTLKAAA

2.3.2 Polyepitope Th Constructs.

(SEQ ID NO: 453) [symbol] [symbol]
KKAVVGILLVVVLGVVFGILIKRRQQKIRKKPICT
IDVYMIMVKCWMIDSEKKAQMRILKETELRKVKVLGSGAKKIKWMALE
SILRRRFTHQSDVKKPICTIDVYMIMVKCWMIDSRKRSHAGYQTI
(PADRE sequence is in bold and Italics; C-terminal
fragment of LAMP-1 is in bold)

2.4 Targeting Sequences

2.4.1 Leader Peptide of Human ErbB2 Protein

(SEQ ID NO: 14)
MELAALCRWGLLLALLPPGAAS

2.4.2 Fragment of Leader Peptide of Human ErbB2 Protein Used in Targeted Polyepitope Constructs

(SEQ ID NO: 13)
MELAALCRWGLLLALLPPGAP

2.4.3 C-Terminal Fragment of Human LAMP-1 Protein (11 Last aa) Used in Targeted Polyepitope Constructs

### RKRSHAGYQTI (SEQ ID NO: 15)

2.4.4 Complete sequences of HLA-DR invariant chain (γ-chain, li)

(SEQ ID NO: 454)
MHRRRSRSCREDQKPVMDDQRDLISNNEQLPMLGRRPGAPESKCSRGALY
TGFSILVTLLLAGQATTAYFLYQQQGRLDKLTVTSQNLQLENLRMKLPKP
PKPVSKMRMATPLLMQALPMGALPQGPMQNATKYGNMTEDHVMHLLQNAD
PLKVYPPLKGSFPENLRHLKNTMETIDWKVFESWMHHWLLFEMSRHSLEQ
KPTDAPPKVLTKCQEEVSHIPAVHPGSFRPKCDENGNYLPLQCYGSIGYC
WCVFPNGTEVPNTRSRGHHNCSESLELEDPSSGLGVTKQDLGPVPM
(immunoregulatory fragment Ii-key is shown in
bold)

2.4.5 Ubiquitin V76

(SEQ ID NO: 455)
MQIFVKTLTGKTITLEVEPSDTIENVKAKIQDKEGIPPDQQRLIFAGKQL
EDGRTLSDYNIQKESTLHLVLRLRGV

2.5 Complete Constructs

2.5.1 Universal Polyepitope Construct:

(SEQ ID NO: 456)
MELAALCRWGLLLALLPPGAPDGENVKIPVAIKVLRENTADGKEECRVLQGLPDGKYSEDPTVPLPDDEAYVMA
GVADLKQETELVEPLTPPDGRASPLTSIISAVVGILLVVVLGVVFPDAGMEHLREVRADGKDIFHKNNQLPDLQ
PEQLQVFRDAQEVQGYVLIPDLAFDGDLGMGAPDLQVIRGRILPDVKVLGSGAFGTVYPIGDLTLGLEPPDLKA
SCVTACPYATLQGLGISWLGLRSLRELGSGLALPMQIAKGMSYALFGPEADQCVPDLKLSYMPIWKFADLKPLQ
RLRIVRGTQLFEDNYALAVARGAPPSTFKAGVVKDVFAFRDLVKITDFGLARLLPLVHRDLAARADVWSYGVTV
RDTTPVTGASPRDLYISAWPDSLRTVCAGGCARSDKIFGSLAFLPDLHCPALVTYADDSTFYRSLLADGKQLMP
YGCLLADGGSCTLVCPL [symbol] [symbol] KKAVVGILLVVVLGVVFGILIKRRQQKIRKKPICTIDVYMIMVK
CWMIDSEKKAQMRILKETELRKVKVLGSGAKKIKWMALESILRRRFTHQSDVKKPICTIDVYMIMVKCWMIDSR
KRSHAGYQTI

2.5.2 HLA-A*0201-Specific Polyepitope Construct:

(SEQ ID NO: 457)
MELAALCRWGLLLALLPPGAPPDLLALLPPGAPDATLEEITGYLAILDEAYVMAPILHNGAYSLPQLFEDNYAL
SIISAVVGIAQLMPYGCLLRLLVVVLGVVRDLQLRSLTEIAILLVVVLGVPDAVVGILLVVADALCRWGLLLAD
YISAWPDSLRDKTFGSLAFL [symbol] [symbol] KKAVVGILLVVVLGVVFGILIKRRQQKIRKKPICTIDVYMI
MVKCWMIDSEKKAQMRILKETELRKVKVLGSGAKKIKWMALESILRRRFTHQSDVKKPICTIDVYMIMVKCWMI
DSRKRSHAGYQTI

2.5.3 HLA-B*3501-Specific Polyepitope Construct:

(SEQ ID NO: 458)
MELAALCRWGLLLALLPPGAPADGKTPTAENPEYAALPASPETHLPILKYSEDPTVPLPDGALPTHDPSPLADNK
EILDEAYADEILDEAYVMPLVVVLGVVFADMQIAKGMSYALMTFGAKPYPLGKAPPPAFSPAFADLHCPALVTY [symbol] [symbol]
KKAVVGILLVVVLGVVFGILIKRRQQKIRKKPICTIDVYMIMVKCWMIDSEKKAQMRILKETELRKVKVLGSG
AKKIKWMALESILRRRFTHQSDVKKPICTIDVYMIMVKCWMIDSRKRSHAGYQTI

2.5.4 Unrelated Protein rHA5 (Corresponding to a Portion (aa 17-346) of Influenza A Virus H5N1 Hemagglutinin (HA) GenBank Accession no. ABL31766)

(SEQ ID NO: 459)
DQICIGYHANNSTEQVDTIMEKNVTVTHAQDILEKTHNGKLCDLDGVKPL
ILRDCSVAGWLLGNPMCDEFINVPEWSYIVEKANPTNDLCYPGSFNDYEE
LKHLLSRINHFEKIQIIPKSSWSDHEASSGVSSACPYLGSPSFFRNVVWL
IKKNSTYPTIKKSYNNTNQEDLLVLWGIHHPNDAAEQTRLYQNPTTYISI
GTSTLNQRLVPKIATRSKVNGQSGRMEFFWAILKPNDAINFESNGNFIAP
EYAYKIVKKGDSAIMKSELEYGNCNTKCQTPMGAINSSMPFHNIHPLTIG
ECPKYVKSNRLVLATGLRNSPQRESRRKKR

### Results and Discussion

To obtain designed polyepitope constructs provided in Examples 2.5.1, 2.5.2 and 2.5.3, corresponding nucleic acids encoding such constructs were produced. The nucleic acid sequences were optimized for expression in human cells by exclusion of rare codons and by minimizing mRNA secondary structure.

The encoding nucleic acids were inserted into pDNA VACC-Ultra plasmid (pDNAVACC5, NBC, USA, http://www.natx.com/). Also, two control plasmids were produced: pHER2-pDNAVACC encoding the full-length HER2 protein (GenBank Accession No. P04626) (positive control) and pDNAVACC-rHA5 encoding an unrelated protein, rHA5, corresponding to a portion (aa 17-346) of hemagglutinin (BA) of Influenza A virus of H5N1 subtype (GenBank Accession No. ABL31766) (negative control). Another negative control was empty plasmid pDNAVACC5.

Four constructs were created and tested:

1. pBCU—pDNAVACC containing the sequence encoding universal polyepitope construct of Example 2.1.3.8;

2. pBCA0201—pDNAVACC containing the sequence encoding polyepitope construct for HLA-A*0201 (3.2-A*0201-Var2);

3. pHER2—pDNAVACC containing the sequence encoding HER2 protein (3.2-B*3501-Var2);

4. prHA5—pDNAVACC containing the sequence encoding a portion of influenza virus H5N1 hemagglutinin (see Example 2.5.4) that is unrelated to HER 2.

A recombinant pQE30 plasmid (Qiagen, Germany) was also created for expression of the common C-terminal fragment of polyepitope constructs (polyECt). This C-terminal fragment was expressed in E. coli cells, purified and used for immunizing animals (BALB/c mice) to generate polyclonal antibodies recognizing polyepitope antigens of the invention. The efficiency of antibody binding was confirmed using ELISA. These antibodies were used to monitor the efficiency of transfection of dendritic cells (DCs) and the efficiency of polyepitope antigen expression after transfection. For detection of HER2 and unrelated protein (rHA5) expression, corresponding polyclonal murine antibodies were used Antibodies were generated by immunizing BALB/c mice i.p. with 20 μg of corresponding antigen (either rHA5 or polyECt) in complete Freund's adjuvant (Sigma, USA) and boosted twice with the same amount of the antigen in incomplete Freund's adjuvant (Sigma, USA) at 14 days integral. Blood was collected 10 days after the last immunization and antiserum was prepared. Each group consisted of six animals, the serum was pooled. Both antigens used for immunization were produced in prokaryotic expression system (E. coli) and purified by affinity chromatography using Ni-NTI agarose (Qiagen, Germany). rHA5 was expressed also using pQE30 expression vector.

The efficiency of induction of T cell response by each of the constructs was determined using the following in vitro assay.

28 healthy donors expressing HLA-A*0201 were selected using PCR assay ALLSET™ GOLD HLA A LOW RES SSP (Invitrogen, USA). This MHC I allomorph is one of the most frequently found in human population. Mononuclear cells (MCs) were fractionated from the peripheral blood of HLA-A2+ normal donors by centrifugation in the ficoll-urografin (Sigma-Aldrich, USA; Schering, Germany) gradient density. Obtained MCs were plated on plastic culture dishes (Nuns, Denmark), and monocyte-enriched adherent cells were observed after a 1-h incubation at 37° C. The nonadherent cells were removed and cryopreserved, and the adherent cells were cultured in the presence of 50 ng/ml rhGM-CSF (BioVision, USA) and 200 ng/ml rhIL-4 (BioVision, USA) in AIM-V medium (Invitrogen, USA) (Obermaier B, et. al, Biol Proud Online, 2003, (5):197-203). After 24 hours LPS (E. coli 055:B5, Sigma, USA) was added (5 μg/ml) to stimulate maturation of DCs. After 24-hour incubation the LPS-treated cells were harvested and used as mature DCs. DCs were labeled using FITC- or PE-conjugated mAb specific to CD3, CD11c, CD14, CD83, CD86, and HLA-DR (all from BD Biosciences, USA). The fluorescence intensity was measured with a FACSCalibur (BD Biosciences, USA). The phagocytosing ability of DCs was assessed using FITC-labeled dextran (Sigma, USA) (Della Bella S. et. al, J. Leukocyte Biol., 2004, 75(11:106-16: Kato M. et. al. Int. Immunol., 2000, 11:1511-1519).

The resulting mature DCs were transfected with the constructs using MATra (Magnet assisted transfection, Promokine, Germany) following producer recommendations (http://www.promokine.info/fileadmin/PDFs/Cell_Transfection/MATra_handbook_PromoKine.pdf). Transfection efficiency was determined using dot-blot analysis (using polyclonal antibodies specific to the common C-terminal portion of polyepitopes of the invention, see above) or using fluorescent microscopy. Fluorescent plasmids were prepared with nick-translation labeling kit (PromoKine, Germany). DCs, transfected with labeled plasmids, were analyzed using fluorescent microscopy. Based on these determinations, efficient transfection and antigen expression was achieved.

The generated mature DCs were co-cultured for 48 hours with previously obtained fractions of autologous non-adherent mononuclear cells (MCs) (in 1:10 ratio) in the presence of recombinant human 40 ng/ml IL-18 and 10 ng/ml IL-12 (BioVision, USA) to stimulate cellular immune response in vitro. Five groups were created:

1. DC:prHA5+non-adherent MCs

2. DC:pHER2+non-adherent MCs

3. DC:pBCU+non-adherent MCs

4. DC:pBCA0201+non-adherent MCs

5. unstimulated non-adherent MCs

To study the T cell response, MCF-7 breast cancer cells (Russian Cell Culture Collection; Institute of Cytology of the Russian Academy of Sciences; Ref. Nos. ECACC 86012803; ICLC HTL95021) were used as target cells (as well as autologous DCs transiently transfected with pHER2). MCF-7 cells express both ErbB2 and HLA-A*0201 (i.e., are HLA-A*0201⁺/ErbB2⁺). This is important, because T-lymphocytes of the majority of selected donors express the same HLA-A allele.

Levels of antigen-specific γIFN and IL-4 production were assayed using intracellular cytokine staining followed by flow cytometry. PBMCs were harvested and resuspended at 2×10⁶ cells/ml in RPMI 1640 and 10% HS. The cultures were restimulated with either MCF-7 cancer cells or autologous DCs, transfected with pHER2 at 2×10⁶ cells/ml. After 2 hours of incubation GolgiPlug™ Protein Transport Inhibitor (containing brefeldin A) solution (BD Bioscienses, USA) was added, and the incubation period was extended to 12 hours at 37° C., 5% CO₂. For intracellular labeling, cells were fixed and permeabilized for 30 min at room temperature using BD FACS Permeabilizing Solution (BD Biosciences, USA) followed by washing. Cells were then labeled with PE- or FITC-conjugated monoclonal antibodies specific to γIFN or IL-4 and CD4 or CD8 (all from BD Biosciences, USA) for 30 min at room temperature in the dark. After washing, stained cells were analyzed by flow cytometry (FACSCalibur, BD Biosciences, USA). (Description of protocol could be found at http://www.bdbiosciences.com/support/resources/protocols/cytokines_fca.jsp).

Induced ex vivo cytotoxic responses were tested by measuring activity of lactate dehydrogenase (LDH) released from lysed target cells (either MCF-7 breast cancer cells or autologous APCs, transfected with pHER2) in different experimental and control groups. The CytoTox 96® Non-Radioactive Cytotoxicity Assay is a colorimetric alternative to radioactive cytotoxicity assays. The CytoTox 96® Assay quantitatively measures lactate dehydrogenase (LDH), a stable cytosolic enzyme that is released upon cell lysis, in much the same way as [⁵¹Cr] is released in radioactive assays. Released LDH in culture supernatants was measured with a 30-minute coupled enzymatic assay that results in the conversion of a tetrazolium salt into a red formazan product. (Description of the protocol could be found in http://www.promega.com/tbs/tb163/tb163.pdf) The amount of color formed is proportional to the number of lysed cells. Visible wavelength absorbance data were collected using multimode microplate reader LB 941 TriStar (Berthold Technologies, Germany). Statistical significance of observed differences between the groups was assessed using Wilcoxon rank-sum test. P<0.05 was considered to be significant.

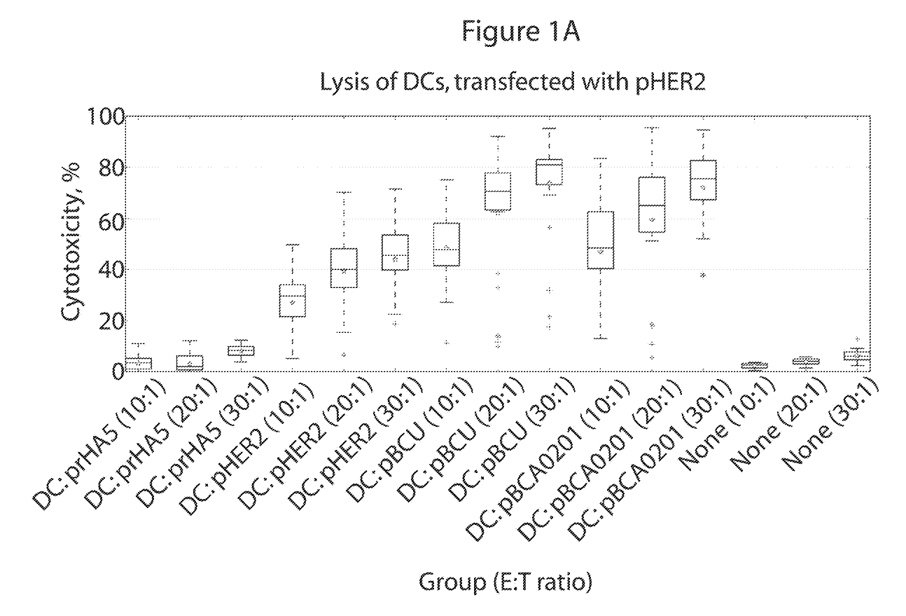

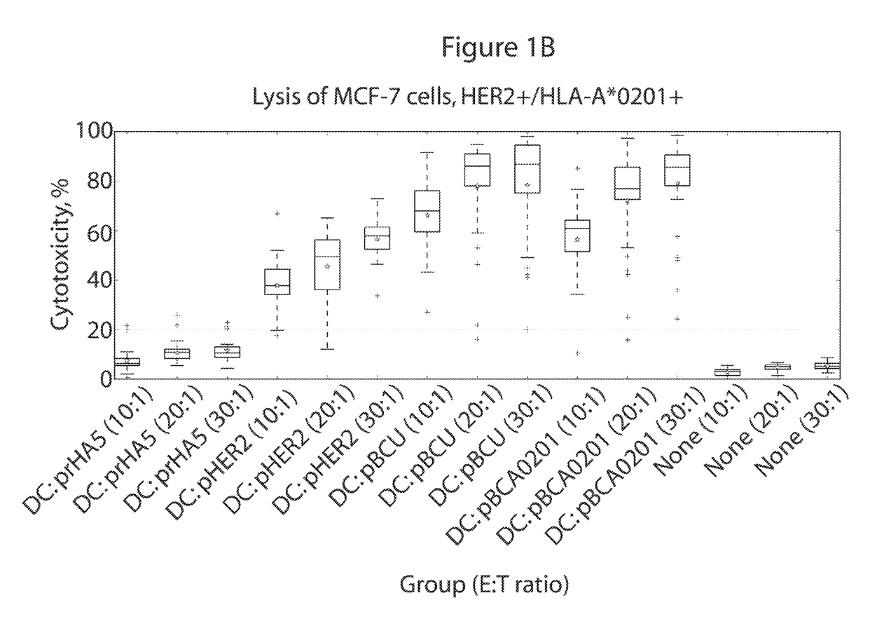

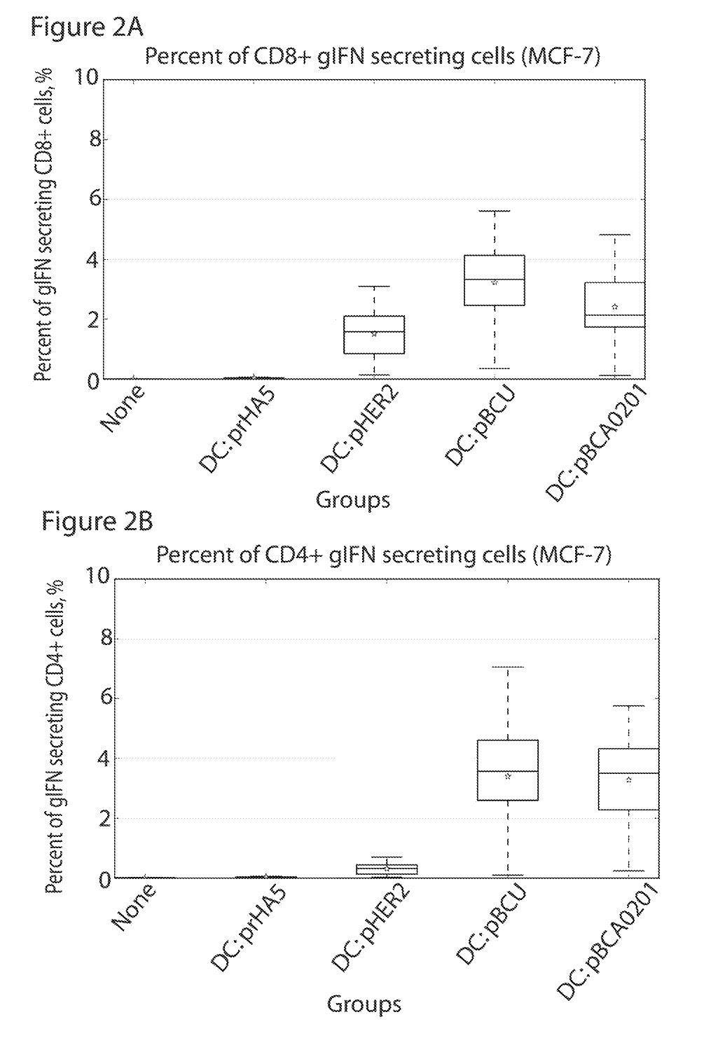

The polyepitope constructs demonstrated higher efficiency of induction of T cell immune responses as compared to the pHER2 construct and the negative control constructs; with the universal construct pBCU demonstrating slightly higher efficiency than the allele-specific construct pBCA0201. Specifically, in the cytotoxicity assays, all experimental groups showed significantly (p<0.001) higher cytotoxicity as compared to both negative controls. In experiments using autologous DCs as target cells (FIG. 1A), there were no statistically significant differences between each of pBCU and pBCA0201 while in both experimental groups cytotocic activity was found to be greater than in corresponding groups of pHER2 (p<0.001); furthermore, when the ratio of effector-to-target cells was ≧20:1 both experimental groups demonstrated superior results as compared to pHER2(30:1) (p<0.01). Using MCF-7 cells as targets (FIG. 1B) revealed that pBCU construct induced slightly higher cytotoxicity than pBCA0201 (with 10:1 effector-to-target ratio the p value was <0.013 and with 20:1-p 0.042; at 30:1 effector-to-target ratio the difference between these two experimental groups was fund to be insignificant). When the ratio of effector-to-target cells was ≧20:1 both experimental groups demonstrated superior results as compared to pHER2(30:1)<0.01). Numbers of γIFN producing CD8+ T-cells, stimulated by the presence of MCF-7 cancer cells, differed significantly between groups stimulated by DCs transfected with pHER2, pBCU and pBCA0201 (p<0.01) (FIG. 2A). Antigen-specific production of γIFN (stimulated by the presence of MCF-7 cells) by CD4+ T-lymphocytes in groups stimulated with DCs transfected with either pBCU or pBCA0201 was found to differ insignificantly, while both these groups demonstrated significantly greater numbers of γIFN-secreting CD4+ T cells than it was found in CD4+ T-cell stimulated with DCs transfected with pHER2 (p<0.001) (FIG. 2B). Thus, the determined in vitro efficiency of the tested constructs for T cell response induction was as follows: pBCU≧pBCA0201>>pHER2.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

It is further to be understood that all values are approximate, and are provided for description.

Patents, patent applications, publications, product descriptions, and protocols are cited throughout this application, the disclosures of which are incorporated herein by reference in their entireties for all purposes.

1-10. (canceled)

11. An isolated polyepitope construct comprising the sequence selected from the group consisting of:
(SEQ ID NO: 86)
CRWGLLLALLVVVLGVVFSIISAVVGIRELGSGLALMELAALCRWADLARDEAYVMAG
VADLVEECRVLQGLADYSEDPTVPLAVKIPVAIKVAQLFEDNYALADVWSYGVTVAW
GLLLALLPATVCAGGCARADIFHKNNQLADASCVTACPYADLLHCPALVTYATELVEPL
TPADLKITDFGLARARGAPPSTFKADLYISAWPDSLAQETELVEPLALQVIRGRILALAAL
CRWGLADLQLMPYGCLLADKIFGSLAFLARGDLTLGLEPAVKVLGSGAFADLVHRDLA
ARADLQPEQLQVFADAFDGDLGMGAAPLQRLRIVRADLRIVRGTQLARASPLTSII;
(SEQ ID NO: 87)
QETELVEPLASCVTACPYADLVKVCRWGLLLALSIISAVVGIAARDEAYVMAGVADLV
KLHCPALVTYARASPLTSIIADLVEECRVLQGLAFDGDLGMGAARGAPPSTFKADLKIFG
SLAFLMELAALCRWADLVQLMPYGCLLAQLFEDNYALKITDFGLARADYISAWPDSLT
VCAGGCARADLWGLLLALLPADLVHRDLAARADLYSEDPTVPLRELGSGLALARGDLT
LGLEPAVKVLGSGAFADLQPEQLQVFADLDVWSYGVTVADLRIVRGTQLAPLQRLRIV
RADLAALCRWGLAVKIPVAIKVADLQVIRGRILALVVVLGVVFADIFHKNNQLATELVE
PLTP;
(SEQ ID NO: 88)
CRWGLLLALASCVTACPYADLYISAWPDSLAVKIPVAIKVAQLFEDNYALADVWSYGV
TVAWGLLLALLPADIFHKNNQLATELVEPLTPADLLHCPALVTYAPLQRLRIVRADLQL
MPYGCLLADKIFGSLAFLMELAALCRWADLVHRDLAARADLQPEQLQVFADAFDGDL
GMGAALQVIRGRILAVKVLGSGAFADLRIVRGTQLARGAPPSTFKADLQETELVEPLRE
LGSGLALLVVVLGVVFSIISAVVGIARGDLTLGLEPADKITDFGLARALAALCRWGLAD
YSEDPTVPLTVCAGGCARARASPLTSIIADLVEECRVLQGLAARDEAYVMAGV;
(SEQ ID NO: 89)
CRWGLLLALAFGPEADQCVADLQLMPYGCLLADYSEDPTVPLAVKIPVAIKVAQLFED
NYALADVWSYGVTVAWGLLLALLPATVCAGGCARAISAVVGILLATLQGLGISWADS
WLGLRSLRADLVKRWGLLLALLLLALLPPGARELGSGLALLVVVLGVVFSIISAVVGIIL
LVVVLGVAIISAVVGILAIKVLRENTADLVQETELVEPLALQVIRGRILAGVVKDVFAFA
DLARDEAYVMAGVADLPLQRLRIVRADLKITDFGLARALGISWLGLRADLQEVQGYVL
IADLHCPALVTYAVKVLGSGAFADGMEHLREVRADTTPVTGASPADASCVTACPYADL
YISAWPDSLARGDLTLGLEPADRGAPPSTFKADLRIVRGTQLATELVEPLTPADAFDGDL
GMGAALAALCRWGLADLQPEQLQVFADAFEDNYALAVAMQIAKGMSYATDFGLARL
LMELAALCRWADLVHRDLAARADGSGAFGTVYARDGENVKIPVADLVDSTFYRSLLA
DLVEECRVLQGLADKIFGSLAFLALCRWGLLLADIFHKNNQLADLSYMPIWKFADLVGS
CTLVCPLARASPLTSIIADLRIVRGTQLF;
(SEQ ID NO: 90)
TTPVTGASPADLSWLGLRSLRADLVGSCTLVCPLAIKVLRENTADYSEDPTVPLMELAA
LCRWADLRWGLLLALLILLVVVLGVADLWGLLLALLPADLVHRDLAARADLDVWSYG
VTVADLGISWLGLRADLVKVQETELVEPLTDFGLARLLRELGSGLALAIISAVVGILAFG
PEADQCVADLVKVCRWGLLLALISAVVGILLGSGAFGTVYADLSYMPIWKFADLVEEC
RVLQGLGVVKDVFAFADLAFEDNYALAVADLKIFGSLAFLASCVTACPYADLVKVQLM
PYGCLLAARDEAYVMAGVADLVKLHCPALVTYAVKVLGSGAFADLQPEQLQVFADLR
IVRGTQLFADLVDSTFYRSLLADGMEHLREVRADLRIVRGTQLATVCAGGCARADLAA
LCRWGLAPLQRLRIVRADLQVIRGRILALVVVLGVVFADIFHKNNQLATLQGLGISWAQ
LFEDNYALARGDLTLGLEPAARDGENVKIPVADLVALCRWGLLLALLALLPPGAARGA
PPSTFKADLKITDFGLARADMQIAKGMSYADAFDGDLGMGAAVKIPVAIKVARASPLTS
IIADLQEVQGYVLIADYISAWPDSLSIISAVVGIATELVEPLTP;
(SEQ ID NO: 91)
CRWGLLLALISAVVGILLAFGPEADQCVADLQETELVEPLTDFGLARLLRELGSGLALLV
VVLGVVFSIISAVVGIILLVVVLGVAIISAVVGILGSGAFGTVYAIKVLRENTADLRIVRGT
QLFADLVKLHCPALVTYAVKVLGSGAFADGMEHLREVRADYISAWPDSLALCRWGLL
LAVKIPVAIKVALAALCRWGLADTTPVTGASPADRGAPPSTFKADLYSEDPTVPLAFDG
DLGMGALLALLPPGAARDGENVKIPVADLVDSTFYRSLLADGSCTLVCPLMELAALCR
WADSWLGLRSLRADLVPLQRLRIVRADLKITDFGLARALGISWLGLRADLQEVQGYVLI
ADKIFGSLAFLASCVTACPYADLRASPLTSIIADLVEECRVLQGLAARDEAYVMAGVAD
LRWGLLLALLGVVKDVFAFADLQLMPYGCLLADLQPEQLQVFADLRIVRGTQLAMQIA
KGMSYADVWSYGVTVAWGLLLALLPATVCAGGCARAQLFEDNYALARGDLTLGLEP
ADIFHKNNQLATELVEPLTPADLVHRDLAARADAFEDNYALAVALQVIRGRILATLQGL
GISWADLSYMPIWKF;
(SEQ ID NO: 92)
TVCAGGCARADGMEHLREVRADGKEECRVLQGLADGRELGSGLALPQLFEDNYALSD
GQETELVEPLPLVVVLGVVFARDGENVKIPVALLALLPPGAAQEVQGYVLIPDLARGDL
TLGLEPAIKVLRENTADAFDGDLGMGAPDAKARDEAYVMAGVADIFHKNNQLAVKVL
GSGAFATLQGLGISWAIAFGPEADQCVPDLKLSYMPIWKFADLKPLQRLRIVRAIISAVV
GILMELAALCRWATGVVKDVFAFADLVKIPVAIKVSIISAVVGIPISAVVGILLPILQPEQL
QVFADGKYSEDPTVPLADMQIAKGMSYARGAPPSTFKADLQVIRGRILPDGRASPLTSII
ADLVHRDLAARADSWLGLRSLRADGKLGISWLGLRADGVKITDFGLARATDFGLARLL
PDGDSTFYRSLLAILLVVVLGVADTTPVTGASPRDLRIVRGTQLATELVEPLTPPDLKAS
CVTACPYPILAALCRWGLADAFEDNYALAVAIDVWSYGVTVAWGLLLALLPRDAKQL
MPYGCLLAIKIFGSLAFLALCRWGLLLRDGRIVRGTQLFADLVGSGAFGTVYADGGSCT
LVCPLPDGYISAWPDSLRDLHCPALVTYALLVCRWGLLLALRWGLLLALL;
(SEQ ID NO: 93)
MELAALCRWGLLLALLPPGAPDGENVKIPVAIKVLRENTADGKEECRVLQGLPDGKYS
EDPTVPLPDDEAYVMAGVADLKQETELVEPLTPPDGRASPLTSIISAVVGILLVVVLGVV
FPDAGMEHLREVRADGKDIFHKNNQLPDLQPEQLQVFRDAQEVQGYVLIPDLAFDGDL
GMGAPDLQVIRGRILPDVKVLGSGAFGTVYPIGDLTLGLEPPDLKASCVTACPYATLQG
LGISWLGLRSLRELGSGLALPMQIAKGMSYALFGPEADQCVPDLKLSYMPIWKFADLKP
LQRLRIVRGTQLFEDNYALAVARGAPPSTFKAGVVKDVFAFRDLVKITDFGLARLLPLV
HRDLAARADVWSYGVTVRDTTPVTGASPRDLYISAWPDSLRTVCAGGCARSDKIFGSL
AFLPDLHCPALVTYADDSTFYRSLLADGKQLMPYGCLLADGGSCTLVCPL;
(SEQ ID NO: 110)
WGLLLALLP-RDA-YSEDPTVPL--ADIDETEYHA-PDLK-AREEGAGSDVFD--
AYGVTVWELM-ALGK-ARDDDDMGDLVD-PLGK-AEITGYLYIS-ADGK-HLDMLRHLY-
ADLK-AHSDCLACLH-AD-LTCSPQPEY-ADLK-QSDVWSYGV-AD-AYKDPPFCVA-PDL-
ARDGDLGMGAA-PIAK-LLDIDETEY-AD-ARDGDPASNTA-AI-ARDGENVKIPV-ALL-
GSGAFGTVY-PD-NASLSFLQD-PLLK-LHCPALVTY-AD-DSTFYRSLL-ADL-
FSPAFDNLY-AILK-TIDVYMIMV;
(SEQ ID NO: 123)
TIDVYMIMV-PDLK-CRWGLLLAL-A-LLALLPPGA-ADG-AILDEAYVMA--ALIHHNTHL-
PDL-RLVHRDLAA--LLLALLPPG-ADGK-QLFEDNYAL-P-ILHNGAYSL-P-SLTLQGLGI-
R-LVDAEEYLV-R-ILLVVVLGV-ADA-SIISAVVGI-A-RLLQETELV-AD-AFEDNYALAV--
AVVGILLVV-A-VVLGVVFGI-AD-ALLNWCMQIA-ADLV-ALCRWGLLL-AD-
YISAWPDSL-RD-KIFGSLAFL-RDL-QLMPYGCLL-ADG-MIMVKCWMI;
(SEQ ID NO: 124)
MELAALCRWGLLLALLPPGAPPDLLALLPPGAPDATLEEITGYLAILDEAYVMAPILHNG
AYSLPQLFEDNYALSIISAVVGIAQLMPYGCLLRLLVVVLGVVRDLQLRSLTEIAILLVVV
LGVPDAVVGILLVVADALCRWGLLLADYISAWPDSLRDKIFGSLAFL;
(SEQ ID NO: 138)
LVPQQGFFC-ADLV-PCARVCYGL-PDLK-KHSDCLACL--ATLEEITGYL-A-
TLSPGKNGV-PDL-DLVDAEEYL-P-ILHNGAYSL-A-SLPDLSVFQ-RD-QIAKGMSYL--
AILDEAYVMA--ALIHHNTHL-AI-AFGPEADQCV-RDLK-LVDAEEYLV-A-QLFEDNYAL--
SIISAVVGI-ADG-THLDMLRHL--ACLTSTVQLV-ADG-FRNPHQALL-ADG-
RLLQETELV-ADL-KIFGSLAFL-A-YISAWPDSL-RD-AYSLTLQGL-RDL-TYLPTNASL-
SDA-RWGLLLALL-A-QLMPYGCLL-ADG-MIMVKCWMI;
(SEQ ID NO: 148)
HYKDPPFCV-AIGK-AIQNEDLGPA-RDL-QIAKGMSYL-A-TLSPGKNGV-SD-
LLALLPPGA-ADG-PYVSRLLGI--AYLSTDVGSC-AD-ILLVVVLGV-ADA-SIISAVVGI-
AD-SLRELGSGL-PTG-RASPLTSII-A-LLVVVLGVV-RDL-AYLTPQGGAA--ALIHHNTHL-
AD-ARPLTSIISAV-ADL-FRNPHQALL-ADGK-KIFGSLAFL--ALLNWCMQIA-ADLK-
ACLTSTVQLV-ADG-YISAWPDSL-A-HLYQGCQVV-ADL-SLTLQGLGI-AD-
QLMPYGCLL-ADG-MIMVKCWMI;
(SEQ ID NO: 156)
CRWGLLLAL-PD-AIQNEDLGPA--AVLDNGDPL-RLLQETELV-ADG-FRNPHQALL-
PDLK-QVFETLEEI-PD-QIAKGMSYL-PD-VVLGVVFGI-ADA-TQLFEDNYA-AD-
AVVGILLVV-AD-RASPLTSII-A-LLVVVLGVV-RD-LQLRSLTEI-A-ILLVVVLGV-ADA-
SIISAVVGI-PD-YVLIAHNQV-AD-VKIPVAIKV--ALIHHNTHL-A-LAALCRWGL-A-
SAVVGILLV-ADGK-KIFGSLAFL-A-IWIPDGENV-AD-TIDVYMIMV-QLMPYGCLL-
ADG-MIMVKCWMI;
(SEQ ID NO: 183)
CVNCSQFLR-AD-LVKSPNHVK-A-ILKETELRK-RDLK-ARILHNGAYS-AD-GVVFGILIK-
ADG-AELMTFGAKP-PDGK-LELTYLPTN-ALGK-KIRKYTMRR-ADLV-LERPKTLSP-A-
VLRENTSPK-A-LLLALLPPG-ADGK-RSLTEILKG--ALLHTANRP-A-ILIKRRQQK-ADGK-
AGILLVVVLG-PDGK-TVWELMTFG-A-ILWKDIFHK-ADGK-RGAPPSTFK-ADL-
QLVTQLMPY-A-VVVLGVVFG-PD-VMAGVGSPY-AILK-LAARNVLVK-ADL-
YTMRRLLQE-ADGK-TFYRSLLED-RD-VVFGILIKR-A-LAFLPESFD-A-YLYISAWPD-
AD-MTFGAKPYD;
(SEQ ID NO: 194)
RWGLLLALL-A-EYVNARHCL-R-DLLEKGERL--AEYHADGGKV-S-DIFHKNNQL-A-
QLFEDNYAL-P-LAALCRWGL-AI-AYGVTVWELM-AI-LRIVRGTQL--ILLVVVLGV-
ADA-TYLPTNASL-A-IWIPDGENV-RLL-VWSYGVTVW-AL-EYLVPQQGF-ADLK-
DVWSYGVTV-PDLK-RFRELVSEF-PDLK-LSYMPIWKF-ADL-SYGVTVWEL-ADA-
QCVNCSQFL-ADAK-VYMIMVKCW-AILK-KWMALESIL-AI-MIMVKCWMI;
(SEQ ID NO: 197)
AWPDSLPDL--DLLEKGERL-RDG-PYVSRLLGI-PDL-TLQGLGISW-A-SLAFLPESF-
PDGK-AVVGILLVV-RT-LVVVLGVVF-A-IWIPDGENV-RLL-VWSYGVTVW-AL-
EYLVPQQGF-ADLK-QLMPYGCLL-AD-SYGVTVWEL-ADL-TYLPTNASL-A-
RIVRGTQLF-RWGLLLALL-A-KWMALESIL-AIGV-VYMIMVKCW;
(SEQ ID NO: 211)
RMARDPQRF-AD-AVRGTQLFED-RD-LQPEQLQVF-ADG-EYVNARHCL-ADA-
RWGLLLALL--ASEGAGSDVF-AGEGLACHQL-PDLK-LQGLGISWL-AI-SYGVTVWEL-
AD-AWPDSLPDL-PL-EYLVPQQGF-ADGK-HNGAYSLTL--AFNHSGICEL-A-
YLVPQQGFF-ADGV-AYSLTLQGL-PDLK-RFRELVSEF-ADGK-ACYGLGMEHL-AL-
VWSYGVTVW-AI-AFQNLQVIRG-ADG-VTVWELMTF-ADGK-AFYRSLLEDD-RDL-
TYLPTNASL-AI-VYMIMVKCW-AILK-KWMALESIL-AD-RFTHQSDVW;
(SEQ ID NO: 224)
CTIDVYMIM-PI-ICELHCPAL-A-QLVTQLMPY-ADG-VSRLLGICL--ALCRWGLLL-PDLK-
ARDEAYVMAGV-AD-ETLEEITGY-A-TEILKGGVL-P-QLFEDNYAL-PD-LQPEQLQVF-
AD-KVPIKWMAL--SIISAVVGI-RD-DTILWKDIF-ALGV-AETHLDMLRH-A-
DVFDGDLGM-PDLK-SLRELGSGL--STVQLVTQL-PLGK-ISWLGLRSL--AFDGDLGMGA-
AD-CRWGLLLAL-PD-VTVWELMTF-ADGK-AFEDNYALAV-RDLK-HTVPWDQLF;
(SEQ ID NO: 239)
LHCPALVTY-SD-LTCSPQPEY-ADL-RLVHRDLAA-ALG-HLDMLRHLY-AD-
LVVVLGVVF-PDGK-DIFHKNNQL-AD-LEEITGYLY-AD-GVVKDVFAF-AD-
ARPGGLRELQL-AD-ETLEEITGY-ALL-THQSDVWSY-AD-AYLEDVRLVH-PDLK-
QVVQGNLEL-AI-GSGAFGTVY-RL-VMAGVGSPY-AILK-LMTFGAKPY-AD-
GTQLFEDNY-ADGK-CVTACPYNY-ADG-GTVYKGIWI-ADL-SMPNPEGRY-ADLK-
HTVPWDQLF-ADLK-SLTLQGLGI-AD-MQIAKGMSY-A-ICLTSTVQL-SD-
DVWSYGVTV-PDLK-MSYLEDVRL-RD-VCTGTDMKL-AD-FSPAFDNLY-AIL-
SPAFDNLYY;
(SEQ ID NO: 258)
KIRKYTMRR-A-YLYISAWPD--LVKSPNHVK-PLLK-KVKVLGSGA-PDG-KETELRKVK-
PD-AIKVLRENT-AD-GGKVPIKWM-ADG-NVKIPVAIK-AD-ARGGCLLDHVRE--
AGLRSLRELG-ADG-RPKTLSPGK-AI-LQRLRIVRG-PDGV-KLRLPASPE-A-
WGLLLALLP-AD-RSRACHPCS-AILK-KRRQQKIRK-ADLK-HVRENRGRL-AD-
ARPGKNGVVKD-A-PLQRLRIVR-RDAK-AARNVLVKS-AD-MARDPQRFV-A-
VLRENTSPK-ADL-VARCPSGVK-ADL-HYKDPPFCV-AD-KIFGSLAFL-A-STFKGTPTA-
ADL-TQRCEKCSK;
(SEQ ID NO: 270)
SMPNPEGRY-ADL-KHSDCLACL--ADMGDLVDAE-RDGK-CVTACPYNY-AL-
GGAVENPEY-AL-AVVKDVFAFG-PLAK-AEIPDLLEKG-PDGK-HLDMLRHLY-ADLK-
TVWELMTFG-AD-LTCSPQPEY-ADL-RSSSTRSGG-ADGK-ETLEEITGY-AD-
VLQGLPREY-AD-ARPLTSIISAV-AL-ASCVTACPY-PLL-SAVVGILLV-ADLV-
AESFDGDPAS-R-DVFDGDLGM-PIL-AAPRSPLAPS-AI-GTQLFEDNY-AIG-
ASLTEILKGG-AD-KGMSYLEDV-AD-VMAGVGSPY-ATLK-SLPDLSVFQ-RDLK-
THQSDVWSY-ADA-SPAFDNLYY-ADL-FSPAFDNLY-ADLK-YYWDQDPPE-ADLV-
LMTFGAKPY;
(SEQ ID NO: 285)
QALLHTANR-AIG-RQVPLQRLR-ADGK-QKIRKYTMR-ADGK-GVGSPYVSR--
RILKETELR-ADL-LEDVRLVHR-ADG-TLIDTNRSR-ADL-GMEHLREVR-ADGK-
REGPLPAAR-RIG-MALESILRR-PDGK-LGISWLGLR-ADGV-KITDFGLAR-A-
PLQRLRIVR-ADG-VVFGILIKR-RDGK-LVHRDLAAR-A-TVCAGGCAR-RDG-
KIRKYTMRR-ADG-AALCRWGLL-ADGK-KIFGSLAFL-PDG-KVPIKWMAL-SD-
ASPLDSTFYR-ADL-VSEFSRMAR-ADLV-CVNCSQFLR-ADLK-LACHQLCAR-AD-
VFQNLQVIR-AIL-SWLGLRSLR;
(SEQ ID NO: 304)
AAPRSPLAPS--ALPAARPAGA-PDG-ALPTHDPSPL-A-ALPASPETHL-SD-
ASPETHLDML--AVLDNGDPL--ASPKANKEIL-P-GAVENPEYL--ASPGKNGVVK-AD-
LPTNASLSF--ADPASNTAPL--AARPAGATL--AAPQPHPPPA-ADGV-LQVIRGRIL-PDG-
RASPLTSII-ADL-APPSPREGPL-RDLK-HVRENRGRL-SDL-AHPPPAFSPA-PDLK-
AMPNQAQMRI-ADLV-RKYTMRRLL-A-GVVKDVFAF-AD-AVPLQRLRIV-ADGK-
GSCTLVCPL-AI-ASPREGPLPA-ADL-RCEKCSKPC;
(SEQ ID NO: 305)
MELAALCRWGLLLALLPPGAPASPKANKEILAARPAGATLALPTHDPSPLAALPASPET
HLSDASPETHLDMLADAPPSPREGPLRDLKHVRENRGRLADLACPSGVKPDLADGSTRS
GGGDLPIASPLTSIISA;
(SEQ ID NO: 319)
YISAWPDSL-PDL-ECRPRFREL-AD-VGILLVVVL-PD-QQKIRKYTM-AD-LFRNPHQAL-
AL-LIKRRQQKI-ADLK-AYGVTVWELM-PDLK-LGMEHLREV--ASPKANKEIL--
ALIHHNTHL-A-DIFHKNNQL-AD-MVHHRHRSS-AD-AVPLQRLRIV-A-ILLVVVLGV-
AD-VSRLLGICL--AFGLARLLDI-AI-LQRLRIVRG-AD-VVGILLVVV-PDG-KVPIKWMAL--
SLAFLPESF-AI-LQVIRGRIL--LVVVLGVVF-A-MRILKETEL-RTG-VLIQRNPQL-PDLK-
ILRRRFTHQ-AD-LAALCRWGL-AD-LDSTFYRSL-RD-LRIVRGTQL-PIAK-ISAVVGILL-
AI-MIMVKCWMI;
(SEQ ID NO: 320)
MELAALCRWGLLLALLPPGAPAIGFHKNNQLALASPKANKEILRDGKDIFHKNNQLPDG
KLGMEHLREVADLFRNPHQALALLGCKKIFGSLPDLRIVRGTQLADGVMRILKETELSD
GQLRSLTEILADGKECRPRFRELADGQLMPYGCLLPDLK;
(SEQ ID NO: 327)
LVVVLGVVF-A-IQRNPQLCY-AILV-TQCVNCSQF-ADG-TLIDTNRSR--ASEGAGSDVF--
ALIHHNTHL-AI-AYGVTVWELM-AIGK-ISWLGLRSL-S-VKVLGSGAF-A-QLFEDNYAL-
PLG-RELGSGLAL--ASCVTACPY-AIL-VTSANIQEF-AIG-VQGNLELTY-AD-
LTCSPQPEY-ADLK-QVVQGNLEL-AI-GSGAFGTVY-RL-VMAGVGSPY-ADGV-
LQVIRGRIL--SLAFLPESF-ADG-VWSYGVTVW-ADA-RIVRGTQLF-WCMQIAKGM-AD-
MQIAKGMSY-A-LMTFGAKPY-RDL-RACHPCSPM;
(SEQ ID NO: 335)
LRIVRGTQL--ASEGAGSDVF--ALDIDETEYH-ADLK-QETELVEPL-AD-ARPEYLTPQGG-
ADGV-EEITGYLYI-PDGK-EECRVLQGL-ADG-RELGSGLAL--AEDLGPASPL-A-
TEILKGGVL-P-LEEITGYLY-PLGK-AGDLGMGAAK-AD-LELTYLPTN-RDG-
VKVLGSGAF-AD-TELVEPLTP-RDLK-SAWPDSLPD-AD-DVWSYGVTV-AD-
MQIAKGMSY-AD-QRFVVIQNE;
(SEQ ID NO: 351)
GRILHNGAY-ADG-CRWGLLLAL--LQPEQLQVF--AILDEAYVMA-RD-AKGLQSLPT-AD-
GRLGSQDLL-ADG-RELGSGLAL--AYLEDVRLVH-RD-AFAGCKKIFG-ADG-
FRNPHQALL-PIGK-AGEGLACHQL-AD-ARPAGATLE-SL-RRLLQETEL--AAGCTGPKH-
AD-AVRGTQLFED-RDLV-RKYTMRRLL-RD-LRIVRGTQL-PDLK-RNPQLCYQD-ADLK-
RQVPLQRLR-ADAK-ARVCYGLGM-ADGV-HRDLAARNV-PD-QRASPLTSI-PLLK-
HRHRSSSTR-ADLV-YLYISAWPD-ADAK-QRFVVIQNE-ADLV-RRQQKIRKY-ADLK-
CRVLQGLPR-ADL-YTMRRLLQE-ADLK-RRFTHQSDV;
(SEQ ID NO: 363)
HTVPWDQLF-ADLV-CRWGLLLAL-RI-ALDIDETEYH-ADL-ARDGDLGMGAA-RD-
LPTNASLSF--ADPASNTAPL--ALPTHDPSPL-AD-NKEILDEAY--ADPAPGAGGM-AI-
AEPLTPSGAM-A-GVVKDVFAF-AD-LTCSPQPEY-ADLK-LVTYNTDTF-AD-
LALLPPGAA-PD-EILDEAYVM-P-LVVVLGVVF--AECVGEGLAC-A-TPTAENPEY-AD-
RSLLEDDDM-ALLV-FVVIQNEDL-AL-AMPNQAQMRI-ADLV-MSYLEDVRL-AI-
LMTFGAKPY-AD-ICELHCPAL-ALGK-YYWDQDPPE-ADL-SPAFDNLYY-ADL-
FSPAFDNLY-AILK-AMPYGCLLDH;
(SEQ ID NO: 364)
MELAALCRWGLLLALLPPGAPADGKTPTAENPEYAALPASPETHLPILKYSEDPTVPLPD
GALPTHDPSPLADNKEILDEAYADEILDEAYVMPLVVVLGVVFADMQIAKGMSYALMT
FGAKPYPLGKAPPPAFSPAFADLHCPALVTY;
(SEQ ID NO: 374)
MELAALCRW--RDLAARNVL-PDA-QETELVEPL--AEEEAPRSPL-PDGK-EECRVLQGL-
ADA-GERLPQPPI-ADG-SETDGYVAP-PDA-AGEGLACHQL-ADG-RELGSGLAL-P-
QLFEDNYAL-PD-ALEDDDMGDL-PDLK-REVRAVTSA--ASEGAGSDVF-A-TEILKGGVL-
PL-EEITGYLYI-PDGK-AENPEYLGL-PDLK-QEVQGYVLI-AD-EQLQVFETL-A-
QVVQGNLEL-A-QEFAGCKKI--ALCRWGLLL-RD-AFEDNYALAV;
(SEQ ID NO: 384)
ISWLGLRSL--AEEEAPRSPL--RDLAARNVL-RLG-GENVKIPVA-RLG-KHSDCLACL-AIG-
GERLPQPPI-ADL-TGTDMKLRL-PDGK-AENPEYLGL-ADG-RELGSGLAL--
REVRAVTSA-ADG-REYVNARHC-A-QEFAGCKKI-A-QETELVEPL-A-TELRKVKVL--
TDMKLRLPA-ADLK-QEVQGYVLI-PDL-ARGGSRCWGESS-ALGV-KITDFGLAR-A-
TDFGLARLL-PDA-RKYTMRRLL-ADG-RELQLRSLT-ADLK-LDSTFYRSL--
MELAALCRW-A-TLQGLGISW-ADL-CQSLTRTVC-ALL-HYKDPPFCV-AIG-
YISAWPDSL-AD-CRWGLLLAL-RDL-TRTVCAGGC-ADLK-TFYRSLLED;
(SEQ ID NO: 389)
TRTVCAGGC-ADG-GGGDLTLGL--ARPEADQCVAC-A-TLQGLGISW-AI-
AFDGDLGMGA-PDAK-ARGDLTLGLEP-PDGK-IDSECRPRF-ADG-VKVLGSGAF-ADG-
QETELVEPL-ADG-RELGSGLAL-A-QEVQGYVLI-ALG-ERGAPPSTF-A-QEFAGCKKI--
MELAALCRW-ALG-VKIPVAIKV-AL-LHCPALVTY;
(SEQ ID NO: 391)
LRIVRGTQL-PIAA-GGGDLTLGL--ARPEADQCVAC-AI-AFDGDLGMGA-PDAK-
ARGDLTLGLEP-PDLK-QETELVEPL-PI-VKVLGSGAF--ASEGAGSDVF-PDG-
RELGSGLAL-A-QEVQGYVLI-ADGK-EECRVLQGL-PDLK-LEEITGYLY-A-TEILKGGVL-
PL-EEITGYLYI-AD-MELAALCRW-AD-ARPDLSVFQNL-ADL-TDFGLARLL-PD-
TRTVCAGGC;
(SEQ ID NO: 403)
CELHCPALV-ADG-GENVKIPVA--ALPASPETHL-RD-ARPEGRYTFGA-ADGK-
IDSECRPRF-ADLK-GERLPQPPI-AIL-AEEAPRSPLA-ADGA-EEITGYLYI--
ALPAARPAGA-PDGK-MEHLREVRA-PDG-RELQLRSLT-ADLK-KEILDEAYV-AT-
AFDGDLGMGA-PDLK-REVRAVTSA--ALPSETDGYV-ADG-AEQRASPLT-ADG-
AGEGLACHQL-ADG-RELGSGLAL-AD-CEKCSKPCA-ADGV-QEVQGYVLI-ADL-
TSANIQEFA-AD-LDSTFYRSL--MELAALCRW-ATGK-AINCTHSCVD-RD-
AFEDNYALAV-RD-LGMGAAKGL--VSRLLGICL-PD-VKIPVAIKV-AI-ASCVTACPY;
(SEQ ID NO: 406)
CRWGLLLAL-PD-ENVKIPVAI--AYGVTVWELM-A-ALPASPETHL--ARPDLSVFQNL-PD-
LPTNASLSF-ADG-ALPTHDPSPL-PDL-ALPSETDGYV-PDLK-LGMEHLREV-AD-
LPQPPICTI-ADGV-QEVQGYVLI-AD-EQLQVFETL-A-LGMGAAKGL-PD-KGMSYLEDV-
A-QEFAGCKKI-S-VGILLVVVL--AMPNQAQMRI-ADLK-LQLRSLTEI-AD-VKIPVAIKV-
A-TDFGLARLL;
(SEQ ID NO: 415)
ASPLDSTFYR-ADG-VENPEYLTP-A-ALPASPETHL--ARAGVGSPYVS-RD-LPTNASLSF-
ADG-ALPTHDPSPL-ADL-LERPKTLSP-AL-AFDGDLGMGA-PDAK-ARGDLTLGLEP-
PDL-ARDDMGDLVDA-PDL-ARPEDECVGE-A-TPTAENPEY-AL-AMPNQAQMRI-ADLK-
LPQPPICTI-AD-ASPLTSIISA-AD-CRWGLLLAL--AGPLPAARPA-PD-AAPRSPLAPS-
ALA-ASPQPEYVNQ-ALG-VKIPVAIKV-AD-ACPSGVKPDL-AD-LHCPALVTY-SDA-
SPAFDNLYY;
(SEQ ID NO: 425)
AWKDIFHKNN-AD-AFDGDLGMGA-PDLK-REVRAVTSA-ALL-AEEAPRSPLA-ADG-
ARDGDPASNTA--ALPAARPAGA-A-IWIPDGENV-SD-LRENTSPKA-RD-LVEPLTPSG-
ADG-LTSIISAVV-A-RKVKVLGSG-ADGV-RELQLRSLT-ADLK-LPQPPICTI-AD-
LQRLRIVRG-PDLK-RGRILHNGA-AD-ASPLTSIISA--ASPLAPSEGA--ACPALVTYNT-AD-
AVPLQRLRIV-ADAA-AMPNQAQMRI-ADLK-AYKDPPFCVA-RDL-AMPIWKFPDE-
ADG-AMPYGCLLDH-ADGK-WGLLLALLP;
(SEQ ID NO: 428)
MELAALCRW-A-VTSANIQEF-ALGK-ENVKIPVAI-ADGK-DIFHKNNQL-RD-
ATLERPKTL--LVVVLGVVF-P-TLQGLGISW-A-DVFDGDLGM-RDLV-ALCRWGLLL-
PDGK-ISWLGLRSL--RSLLEDDDM-ADG-GSGAFGTVY-ADA-GTQLFEDNY-RDLK-
LSYMPIWKF-ADLK-PAFDNLYYW-ADL-QLMPYGCLL-PDLK-MSYLEDVRL-R-
DVWSYGVTV-PDLK-RFTHQSDVW-ADLV-HTVPWDQLF;
(SEQ ID NO: 436)
PAFDNLYYW-AIL-CTIDVYMIM-ADLV-RMARDPQRF-AD-KGCPAEQRA-PDLK-
LGSQDLLNW--AIISAVVGIL-AL-RCEKCSKPC-AIL-VTSANIQEF-ADL-GAMPNQAQM-
AD-AVTGASPGGL-P-ISAVVGILL-PD-RSGGGDLTL--AYLSTDVGSC-A-LAALCRWGL-
AL-ASCVTACPY-ADL-HTVPWDQLF-ADLK-LSYMPIWKF-ADG-RASPLTSII-ADG-
VTVWELMTF-ADGV-ARGQECVEEC-ADL-RIVRGTQLF-TRTVCAGGC-AD-
KIFGSLAFL-PD-VCTGTDMKL-AD-LCYQDTILW,
and
(SEQ ID NO: 453)
AKFVAAWTLKAAAKKAVVGILLVVVLGVVFGILIKRRQQKIRKKPICTIDVYMIMVKC
WMIDSEKKAQMRILKETELRKVKVLGSGAKKIKWMALESILRRRFTHQSDVKKPICTID
VYMIMVKCWMIDSRKRSHAGYQTI.

12. An isolated polyepitope construct consisting of the sequence selected from the group consisting of:
(SEQ ID NO: 456-universal)
MELAALCRWGLLLALLPPGAPDGENVKIPVAIKVLRENTADGKEECRVLQ
GLPDGKYSEDPTVPLPDDEAYVMAGVADLKQETELVEPLTPPDGRASPLT
SIISAVVGILLVVVLGVVFPDAGMEHLREVRADGKDIFHKNNQLPDLQPE
QLQVFRDAQEVQGYVLIPDLAFDGDLGMGAPDLQVIRGRILPDVKVLGSG
AFGTVYPIGDLTLGLEPPDLKASCVTACPYATLQGLGISWLGLRSLRELG
SGLALPMQIAKGMSYALFGPEADQCVPDLKLSYMPIWKFADLKPLQRLRI
VRGTQLFEDNYALAVARGAPPSTFKAGVVKDVFAFRDLVKITDFGLARLL
PLVHRDLAARADVWSYGVTVRDTTPVTGASPRDLYISAWPDSLRTVCAGG
CARSDKIFGSLAFLPDLHCPALVTYADDSTFYRSLLADGKQLMPYGCLLA
DGGSCTLVCPLAKFVAAWTLKAAAKKAVVGILLVVVLGVVFGILIKRRQQ
KIRKKPICTIDVYMIMVKCWMIDSEKKAQMRILKETELRKVKVLGSGAKK
IKWMALESILRRRFTHQSDVKKPICTIDVYMIMVKCWMIDSRKRSHAGYQ
TI,
(SEQ ID NO: 457-HLA-A*0201-specific)
MELAALCRWGLLLALLPPGAPPDLLALLPPGAPDATLEEITGYLAILDEA
YVMAPILHNGAYSLPQLFEDNYALSIISAVVGIAQLMPYGCLLRLLVVVL
GVVRDLQLRSLTEIAILLVVVLGVPDAVVGILLVVADALCRWGLLLADYI
SAWPDSLRDKIFGSLAFLAKFVAAWTLKAAAKKAVVGILLVVVLGVVFGI
LIKRRQQKIRKKPICTIDVYMIMVKCWMIDSEKKAQMRILKETELRKVKV
LGSGAKKIKWMALESILRRRFTHQSDVKKPICTIDVYMIMVKCWMIDSRK
RSHAGYQTI,
and
(SEQ ID NO: 458-HLA-B*3501-specific)
MELAALCRWGLLLALLPPGAPADGKTPTAENPEYAALPASPETHLPILKY
SEDPTVPLPDGALPTHDPSPLADNKEILDEAYADEILDEAYVMPLVVVLG
VVFADMQIAKGMSYALMTFGAKPYPLGKAPPPAFSPAFADLHCPALVTYA
KFVAAWTLKAAAKKAVVGILLVVVLGVVFGILIKRRQQKIRKKPICTIDV
YMIMVKCWMIDSEKKAQMRILKETELRKVKVLGSGAKKIKWMALESILRR
RFTHQSDVKKPICTIDVYMIMVKCWMIDSRKRSHAGYQTI.

13-14. (canceled)

15. A pharmaceutical composition comprising one or more polyepitope constructs of claim 11 and a pharmaceutically acceptable carrier or excipient.

16. An isolated nucleic acid encoding one or more polyepitope constructs of claim 11.

17. A pharmaceutical composition comprising the nucleic acid of claim 16 and a pharmaceutically acceptable carrier or excipient.

18. (canceled)

19. A method for inducing a T cell response in a mammal comprising administering to said mammal the pharmaceutical composition of claim 15.

20. A method for inducing a T cell response in a mammal comprising administering to said mammal the pharmaceutical composition of claim 17.

21. A method for treating a breast cancer in a mammal comprising administering to said mammal the pharmaceutical composition of claim 15.

22. A method for treating a breast cancer in a mammal comprising administering to said mammal the pharmaceutical composition of claim 17.

23. A method for inducing a T cell response in a mammal comprising administering to said mammal one or more polyepitope constructs of claim 11.

24. A method for inducing a T cell response in a mammal comprising administering to said mammal the nucleic acid of claim 16.

25. A method for treating a breast cancer in a mammal comprising administering to said mammal one or more polyepitope constructs of claim 11.

26. A method for treating a breast cancer in a mammal comprising administering to said mammal the nucleic acid of claim 16.

27. A pharmaceutical composition comprising one or more polyepitope constructs of claim 12 and a pharmaceutically acceptable carrier or excipient.

28. An isolated nucleic acid encoding one or more polyepitope constructs of claim 12.

29. A pharmaceutical composition comprising the nucleic acid of claim 28 and a pharmaceutically acceptable carrier or excipient.

30. A method for inducing a T cell response in a mammal comprising administering to said mammal the pharmaceutical composition of claim 27.

31. A method for inducing a T cell response in a mammal comprising administering to said mammal the pharmaceutical composition of claim 29.

32. A method for treating a breast cancer in a mammal comprising administering to said mammal the pharmaceutical composition of claim 27.

33. A method for treating a breast cancer in a mammal comprising administering to said mammal the pharmaceutical composition of claim 29.

34. A method for inducing a T cell response in a mammal comprising administering to said mammal one or more polyepitope constructs of claim 12.

35. A method for inducing a T cell response in a mammal comprising administering to said mammal the nucleic acid of claim 28.

36. A method for treating a breast cancer in a mammal comprising administering to said mammal one or more polyepitope constructs of claim 12.

37. A method for treating a breast cancer in a mammal comprising administering to said mammal the nucleic acid of claim 28.

38. The method of claim 32, wherein the breast cancer is a HER2-positive breast cancer.

39. The method of claim 33, wherein the breast cancer is a HER2-positive breast cancer.

40. The method of claim 36, wherein the breast cancer is a HER2-positive breast cancer.

41. The method of claim 37, wherein the breast cancer is a HER2-positive breast cancer.


SEQUENCE LISTING (464 sequences)

SEQ ID NO: 1 - protein, 13 aa, Artificial Sequence
  source: /note=“Description of Artificial Sequence Synthetic peptide”
AKFVAAWTLKAAA

SEQ ID NO: 2 - protein, 6 aa, Artificial Sequence
  source: /note=“Description of Artificial Sequence Synthetic peptide”
ADLVKV

SEQ ID NO: 3 - protein, 6 aa, Artificial Sequence
  source: /note=“Description of Artificial Sequence Synthetic peptide”
ADLVAG

SEQ ID NO: 4 - protein, 6 aa, Artificial Sequence
  source: /note=“Description of Artificial Sequence Synthetic peptide”
ADLAVK

SEQ ID NO: 5 - protein, 4 aa, Artificial Sequence
  source: /note=“Description of Artificial Sequence Synthetic peptide”
ADLV

SEQ ID NO: 6 - protein, 5 aa, Artificial Sequence
  source: /note=“Description of Artificial Sequence Synthetic peptide”
ADLVK

SEQ ID NO: 7 - protein, 27 aa, Artificial Sequence
  source: /note=“Description of Artificial Sequence Synthetic peptide”
AVVGILLVVVLGVVFGILIKRRQQKIR

SEQ ID NO: 8 - protein, 20 aa, Artificial Sequence
  source: /note=“Description of Artificial Sequence Synthetic peptide”
PICTIDVYMIMVKCWMIDSE

SEQ ID NO: 9 - protein, 21 aa, Artificial Sequence
  source: /note=“Description of Artificial Sequence Synthetic peptide”
AQMRILKETELRKVKVLGSGA

SEQ ID NO: 10 - protein, 20 aa, Artificial Sequence
  source: /note=“Description of Artificial Sequence Synthetic peptide”
IKWMALESILRRRFTHQSDV

SEQ ID NO: 11 - protein, 19 aa, Artificial Sequence
  source: /note=“Description of Artificial Sequence Synthetic peptide”
PICTIDVYMIMVKCWMIDS

SEQ ID NO: 12 - protein, 130 aa, Artificial Sequence
  source: /note=“Description of Artificial Sequence Synthetic polypeptide”
AKFVAAWTLKAAAKKAVVGILLVVVLGVVFGILIKRRQQKIRKKPICTIDVYMIMVKCWMIDSEKKAQMRILKETELRKVKVLGSGAKKIKWMALESILRRRFTHQSDVKKPICTIDVYMIMVKCWMIDS

SEQ ID NO: 13 - protein, 21 aa, Artificial Sequence
  source: /note=“Description of Artificial Sequence Synthetic peptide”
MELAALCRWGLLLALLPPGAP

SEQ ID NO: 14 - protein, 22 aa, Artificial Sequence
  source: /note=“Description of Artificial Sequence Synthetic peptide”
MELAALCRWGLLLALLPPGAAS

SEQ ID NO: 15 - protein, 11 aa, Artificial Sequence
  source: /note=“Description of Artificial Sequence Synthetic peptide”
RKRSHAGYQTI

SEQ ID NO: 16 - protein, 34 aa, Artificial Sequence
  source: /note=“Description of Artificial Sequence Synthetic polypeptide”
IPIAVGGALAGLVLIVLIAYLVGRKRSHAGYQTI

SEQ ID NO: 17 - protein, 16 aa, Artificial Sequence
  source: /note=“Description of Artificial Sequence Synthetic peptide”
LRMKLPKPPKPVSQMR

SEQ ID NO: 18 - protein, 7 aa, Artificial Sequence
  source: /note=“Description of Artificial Sequence Synthetic peptide”
LRMKLPK

SEQ ID NO: 19 - protein, 4 aa, Artificial Sequence
  source: /note=“Description of Artificial Sequence Synthetic peptide”
LRMK

SEQ ID NO: 20 - protein, 1255 aa, Artificial Sequence [sequence omitted]

SEQ ID NO: 21 - protein, 9 aa, Artificial Sequence
  source: /note=“Description of Artificial Sequence Synthetic peptide”
CRWGLLLAL

SEQ ID NO: 22 - protein, 9 aa, Artificial Sequence
  source: /note=“Description of Artificial Sequence Synthetic peptide”
LAALCRWGL

SEQ ID NO: 23 - protein, 9 aa, Artificial Sequence
  source: /note=“Description of Artificial Sequence Synthetic peptide”
RELGSGLAL

SEQ ID NO: 24 - protein, 9 aa, Artificial Sequence
  source: /note=“Description of Artificial Sequence Synthetic peptide”
WGLLLALLP

SEQ ID NO: 25 - protein, 9 aa, Artificial Sequence
  source: /note=“Description of Artificial Sequence Synthetic peptide”
LVVVLGVVF

SEQ ID NO: 26 - protein, 9 aa, Artificial Sequence
  source: /note=“Description of Artificial Sequence Synthetic peptide”
KITDFGLAR

SEQ ID NO: 27 - protein, 9 aa, Artificial Sequence
  source: /note=“Description of Artificial Sequence Synthetic peptide”
QLFEDNYAL

SEQ ID NO: 28 - protein, 9 aa, Artificial Sequence
  source: /note=“Description of Artificial Sequence Synthetic peptide”
YISAWPDSL

SEQ ID NO: 29 - protein, 9 aa, Artificial Sequence
  source: /note=“Description of Artificial Sequence Synthetic peptide”
GDLTLGLEP

SEQ ID NO: 30 - protein, 9 aa, Artificial Sequence
  source: /note=“Description of Artificial Sequence Synthetic peptide”
DVWSYGVTV

SEQ ID NO: 31 - protein, 9 aa, Artificial Sequence
  source: /note=“Description of Artificial Sequence Synthetic peptide”
KIFGSLAFL

SEQ ID NO: 32 - protein, 9 aa, Artificial Sequence
  source: /note=“Description of Artificial Sequence Synthetic peptide”
FDGDLGMGA

SEQ ID NO: 33 - protein, 9 aa, Artificial Sequence
  source: /note=“Description of Artificial Sequence Synthetic peptide”
LVHRDLAAR

SEQ ID NO: 34 - protein, 9 aa, Artificial Sequence
  source: /note=“Description of Artificial Sequence Synthetic peptide”
MELAALCRW

SEQ ID NO: 35 - protein, 9 aa, Artificial Sequence
  source: /note=“Description of Artificial Sequence Synthetic peptide”
RASPLTSII

SEQ ID NO: 36 - protein, 9 aa, Artificial Sequence
  source: /note=“Description of Artificial Sequence Synthetic peptide”
RGAPPSTFK

SEQ ID NO: 37 - protein, 9 aa, Artificial Sequence
  source: /note=“Description of Artificial Sequence Synthetic peptide”
SIISAVVGI

SEQ ID NO: 38 - protein, 9 aa, Artificial Sequence
  source: /note=“Description of Artificial Sequence Synthetic peptide”
LHCPALVTY

SEQ ID NO: 39 - protein, 9 aa, Artificial Sequence
  source: /note=“Description of Artificial Sequence Synthetic peptide”
LRIVRGTQL

SEQ ID NO: 40 - protein, 9 aa, Artificial Sequence
  source: /note=“Description of Artificial Sequence Synthetic peptide”
VKVLGSGAF

SEQ ID NO: 41 - protein, 9 aa, Artificial Sequence
  source: /note=“Description of Artificial Sequence Synthetic peptide”
LQPEQLQVF

SEQ ID NO: 42 - protein, 9 aa, Artificial Sequence
  source: /note=“Description of Artificial Sequence Synthetic peptide”
VKIPVAIKV

SEQ ID NO: 43 - protein, 9 aa, Artificial Sequence
  source: /note=“Description of Artificial Sequence Synthetic peptide”
QLMPYGCLL

SEQ ID NO: 44 - protein, 9 aa, Artificial Sequence
  source: /note=“Description of Artificial Sequence Synthetic peptide”
QETELVEPL

SEQ ID NO: 45 - protein, 9 aa, Artificial Sequence
  source: /note=“Description of Artificial Sequence Synthetic peptide”
DIFHKNNQL

SEQ ID NO: 46 - protein, 9 aa, Artificial Sequence
  source: /note=“Description of Artificial Sequence Synthetic peptide”
ASCVTACPY

SEQ ID NO: 47 - protein, 9 aa, Artificial Sequence
  source: /note=“Description of Artificial Sequence Synthetic peptide”
TELVEPLTP

SEQ ID NO: 48 - protein, 9 aa, Artificial Sequence
  source: /note=“Description of Artificial Sequence Synthetic peptide”
PLQRLRIVR

SEQ ID NO: 49 - protein, 9 aa, Artificial Sequence
  source: /note=“Description of Artificial Sequence Synthetic peptide”
LQVIRGRIL

SEQ ID NO: 50 - protein, 9 aa, Artificial Sequence
  source: /note=“Description of Artificial Sequence Synthetic peptide”
DEAYVMAGV

SEQ ID NO: 51 - protein, 9 aa, Artificial Sequence
  source: /note=“Description of Artificial Sequence Synthetic peptide”
EECRVLQGL

SEQ ID NO: 52 - protein, 9 aa, Artificial Sequence
  source: /note=“Description of Artificial Sequence Synthetic peptide”
TVCAGGCAR

SEQ ID NO: 53 - protein, 9 aa, Artificial Sequence
  source: /note=“Description of Artificial Sequence Synthetic peptide”
YSEDPTVPL

SEQ ID NO: 54 - protein, 9 aa, Artificial Sequence
  source: /note=“Description of Artificial Sequence Synthetic peptide”
RWGLLLALL

SEQ ID NO: 55 - protein, 9 aa, Artificial Sequence
  source: /note=“Description of Artificial Sequence Synthetic peptide”
FEDNYALAV

SEQ ID NO: 56 - protein, 9 aa, Artificial Sequence
  source: /note=“Description of Artificial Sequence Synthetic peptide”
QEVQGYVLI

SEQ ID NO: 57 - protein, 9 aa, Artificial Sequence
  source: /note=“Description of Artificial Sequence Synthetic peptide”
LLALLPPGA

SEQ ID NO: 58 - protein, 9 aa, Artificial Sequence
  source: /note=“Description of Artificial Sequence Synthetic peptide”
GSGAFGTVY

SEQ ID NO: 59 - protein, 9 aa, Artificial Sequence
  source: /note=“Description of Artificial Sequence Synthetic peptide”
LGISWLGLR

SEQ ID NO: 60 - protein, 9 aa, Artificial Sequence
  source: /note=“Description of Artificial Sequence Synthetic peptide”
ISAVVGILL

SEQ ID NO: 61 - protein, 9 aa, Artificial Sequence
  source: /note=“Description of Artificial Sequence Synthetic peptide”
MQIAKGMSY

SEQ ID NO: 62 - protein, 9 aa, Artificial Sequence
  source: /note=“Description of Artificial Sequence Synthetic peptide”
LSYMPIWKF

SEQ ID NO: 63 - protein, 9 aa, Artificial Sequence
  source: /note=“Description of Artificial Sequence Synthetic peptide”
GVVKDVFAF

SEQ ID NO: 64 - protein, 9 aa, Artificial Sequence
  source: /note=“Description of Artificial Sequence Synthetic peptide”
AIKVLRENT

SEQ ID NO: 65 - protein, 9 aa, Artificial Sequence
  source: /note=“Description of Artificial Sequence Synthetic peptide”
SWLGLRSLR

SEQ ID NO: 66 - protein, 9 aa, Artificial Sequence
  source: /note=“Description of Artificial Sequence Synthetic peptide”
ILLVVVLGV

SEQ ID NO: 67 - protein, 9 aa, Artificial Sequence
  source: /note=“Description of Artificial Sequence Synthetic peptide”
FGPEADQCV

SEQ ID NO: 68 - protein, 9 aa, Artificial Sequence
  source: /note=“Description of Artificial Sequence Synthetic peptide”
TLQGLGISW

SEQ ID NO: 69 - protein, 9 aa, Artificial Sequence
  source: /note=“Description of Artificial Sequence Synthetic peptide”
TDFGLARLL

SEQ ID NO: 70 - protein, 9 aa, Artificial Sequence
  source: /note=“Description of Artificial Sequence Synthetic peptide”
DSTFYRSLL

SEQ ID NO: 71 - protein, 9 aa, Artificial Sequence
  source: /note=“Description of Artificial Sequence Synthetic peptide”
IISAVVGIL

SEQ ID NO: 72 - protein, 9 aa, Artificial Sequence
  source: /note=“Description of Artificial Sequence Synthetic peptide”
TTPVTGASP

SEQ ID NO: 73 - protein, 9 aa, Artificial Sequence
  source: /note=“Description of Artificial Sequence Synthetic peptide”
GMEHLREVR

SEQ ID NO: 74 - protein, 9 aa, Artificial Sequence
  source: /note=“Description of Artificial Sequence Synthetic peptide”
ALCRWGLLL

SEQ ID NO: 75 - protein, 9 aa, Artificial Sequence
  source: /note=“Description of Artificial Sequence Synthetic peptide”
RIVRGTQLF

SEQ ID NO: 76 - protein, 9 aa, Artificial Sequence
  source: /note=“Description of Artificial Sequence Synthetic peptide”
GSCTLVCPL

SEQ ID NO: 77 - protein, 9 aa, Artificial Sequence
  source: /note=“Description of Artificial Sequence Synthetic peptide”
DGENVKIPV

SEQ ID NO: 78 - protein, 20 aa, Artificial Sequence
  source: /note=“Description of Artificial Sequence Synthetic peptide”
MELAALCRWGLLLALLPPGA

SEQ ID NO: 79 - protein, 22 aa, Artificial Sequence
  source: /note=“Description of Artificial Sequence Synthetic peptide”
PLQRLRIVRGTQLFEDNYALAV

SEQ ID NO: 80 - protein, 24 aa, Artificial Sequence
  source: /note=“Description of Artificial Sequence Synthetic peptide”
TLQGLGISWLGLRSLRELGSGLAL

SEQ ID NO: 81 - protein, 25 aa, Artificial Sequence
  source: /note=“Description of Artificial Sequence Synthetic peptide”
RASPLTSIISAVVGILLVVVLGVVF

SEQ ID NO: 82 - protein, 11 aa, Artificial Sequence
  source: /note=“Description of Artificial Sequence Synthetic peptide”
QETELVEPLTP

SEQ ID NO: 83 - protein, 13 aa, Artificial Sequence
  source: /note=“Description of Artificial Sequence Synthetic peptide”
VKVLGSGAFGTVY

SEQ ID NO: 84 - protein, 18 aa, Artificial Sequence
  source: /note=“Description of Artificial Sequence Synthetic peptide”
DGENVKIPVAIKVLRENT

SEQ ID NO: 85 - protein, 11 aa, Artificial Sequence
  source: /note=“Description of Artificial Sequence Synthetic peptide”
KITDFGLARLL

SEQ ID NO: 86 - protein, 349 aa, Artificial Sequence
  source: /note=“Description of Artificial Sequence Synthetic polypeptide”
CRWGLLLALLVVVLGVVFSIISAVVGIRELGSGLALMELAALCRWADLARDEAYVMAGVADLVEECRVLQGLADYSEDPTVPLAVKIPVAIKVAQLFEDNYALADVWSYGVTVAWGLLLALLPATVCAGGCARADIFHKNNQLADASCVTACPYADLLHCPALVTYATELVEPLTPADLKITDFGLARARGAPPSTFKADLYISAWPDSLAQETELVEPLALQVIRGRILALAALCRWGLADLQLMPYGCLLADKIFGSLAFLARGDLTLGLEPAVKVLGSGAFADLVHRDLAARADLQPEQLQVFADAFDGDLGMGAAPLQRLRIVRADLRIVRGTQLARASPLTSII

SEQ ID NO: 87 - protein, 355 aa, Artificial Sequence
  source: /note=“Description of Artificial Sequence Synthetic polypeptide”
QETELVEPLASCVTACPYADLVKVCRWGLLLALSIISAVVGIAARDEAYVMAGVADLVKLHCPALVTYARASPLTSIIADLVEECRVLQGLAFDGDLGMGAARGAPPSTFKADLKIFGSLAFLMELAALCRWADLVQLMPYGCLLAQLFEDNYALKITDFGLARADYISAWPDSLTVCAGGCARADLWGLLLALLPADLVHRDLAARADLYSEDPTVPLRELGSGLALARGDLTLGLEPAVKVLGSGAFADLQPEQLQVFADLDVWSYGVTVADLRIVRGTQLAPLQRLRIVRADLAALCRWGLAVKIPVAIKVADLQVIRGRILALVVVLGVVFADIFHKNNQLATELVEPLTP

SEQ ID NO: 88 - protein, 345 aa, Artificial Sequence
  source: /note=“Description of Artificial Sequence Synthetic polypeptide”
CRWGLLLALASCVTACPYADLYISAWPDSLAVKIPVAIKVAQLFEDNYALADVWSYGVTVAWGLLLALLPADIFHKNNQLATELVEPLTPADLLHCPALVTYAPLQRLRIVRADLQLMPYGCLLADKIFGSLAFLMELAALCRWADLVHRDLAARADLQPEQLQVFADAFDGDLGMGAALQVIRGRILAVKVLGSGAFADLRIVRGTQLARGAPPSTFKADLQETELVEPLRELGSGLALLVVVLGVVFSIISAVVGIARGDLTLGLEPADKITDFGLARALAALCRWGLADYSEDPTVPLTVCAGGCARARASPLTSIIADLVEECRVLQGLAARDEAYVMAGV

SEQ ID NO: 89 - protein, 612 aa, Artificial Sequence
  source: /note=“Description of Artificial Sequence Synthetic polypeptide”
CRWGLLLALAFGPEADQCVADLQLMPYGCLLADYSEDPTVPLAVKIPVAIKVAQLFEDNYALADVWSYGVTVAWGLLLALLPATVCAGGCARAISAVVGILLATLQGLGISWADSWLGLRSLRADLVKRWGLLLALLLLALLPPGARELGSGLALLVVVLGVVFSIISAVVGIILLVVVLGVAIISAVVGILAIKVLRENTADLVQETELVEPLALQVIRGRILAGVVKDVFAFADLARDEAYVMAGVADLPLQRLRIVRADLKITDFGLARALGISWLGLRADLQEVQGYVLIADLHCPALVTYAVKVLGSGAFADGMEHLREVRADTTPVTGASPADASCVTACPYADLYISAWPDSLARGDLTLGLEPADRGAPPSTFKADLRIVRGTQLATELVEPLTPADAFDGDLGMGAALAALCRWGLADLQPEQLQVFADAFEDNYALAVAMQIAKGMSYATDFGLARLLMELAALCRWADLVHRDLAARADGSGAFGTVYARDGENVKIPVADLVDSTFYRSLLADLVEECRVLQGLADKIFGSLAFLALCRWGLLLADIFHKNNQLADLSYMPIWKFADLVGSCTLVCPLARASPLTSIIADLRIVRGTQLF

SEQ ID NO: 90 - protein, 627 aa, Artificial Sequence
  source: /note=“Description of Artificial Sequence Synthetic polypeptide”
TTPVTGASPADLSWLGLRSLRADLVGSCTLVCPLAIKVLRENTADYSEDPTVPLMELAALCRWADLRWGLLLALLILLVVVLGVADLWGLLLALLPADLVHRDLAARADLDVWSYGVTVADLGISWLGLRADLVKVQETELVEPLTDFGLARLLRELGSGLALAIISAVVGILAFGPEADQCVADLVKVCRWGLLLALISAVVGILLGSGAFGTVYADLSYMPIWKFADLVEECRVLQGLGVVKDVFAFADLAFEDNYALAVADLKIFGSLAFLASCVTACPYADLVKVQLMPYGCLLAARDEAYVMAGVADLVKLHCPALVTYAVKVLGSGAFADLQPEQLQVFADLRIVRGTQLFADLVDSTFYRSLLADGMEHLREVRADLRIVRGTQLATVCAGGCARADLAALCRWGLAPLQRLRIVRADLQVIRGRILALVVVLGVVFADIFHKNNQLATLQGLGISWAQLFEDNYALARGDLTLGLEPAARDGENVKIPVADLVALCRWGLLLALLALLPPGAARGAPPSTFKADLKITDFGLARADMQIAKGMSYADAFDGDLGMGAAVKIPVAIKVARASPLTSIIADLQEVQGYVLIADYISAWPDSLSIISAVVGIATELVEPLTP

SEQ ID NO: 91 - protein, 602 aa, Artificial Sequence
  source: /note=“Description of Artificial Sequence Synthetic polypeptide”
CRWGLLLALISAVVGILLAFGPEADQCVADLQETELVEPLTDFGLARLLRELGSGLALLVVVLGVVFSIISAVVGIILLVVVLGVAIISAVVGILGSGAFGTVYAIKVLRENTADLRIVRGTQLFADLVKLHCPALVTYAVKVLGSGAFADGMEHLREVRADYISAWPDSLALCRWGLLLAVKIPVAIKVALAALCRWGLADTTPVTGASPADRGAPPSTFKADLYSEDPTVPLAFDGDLGMGALLALLPPGAARDGENVKIPVADLVDSTFYRSLLADGSCTLVCPLMELAALCRWADSWLGLRSLRADLVPLQRLRIVRADLKITDFGLARALGISWLGLRADLQEVQGYVLIADKIFGSLAFLASCVTACPYADLRASPLTSIIADLVEECRVLQGLAARDEAYVMAGVADLRWGLLLALLGVVKDVFAFADLQLMPYGCLLADLQPEQLQVFADLRIVRGTQLAMQIAKGMSYADVWSYGVTVAWGLLLALLPATVCAGGCARAQLFEDNYALARGDLTLGLEPADIFHKNNQLATELVEPLTPADLVHRDLAARADAFEDNYALAVALQVIRGRILATLQGLGISWADLSYMPIWKF

SEQ ID NO: 92 - protein, 639 aa, Artificial Sequence
  source: /note=“Description of Artificial Sequence Synthetic polypeptide”
TVCAGGCARADGMEHLREVRADGKEECRVLQGLADGRELGSGLALPQLFEDNYALSDGQETELVEPLPLVVVLGVVFARDGENVKIPVALLALLPPGAAQEVQGYVLIPDLARGDLTLGLEPAIKVLRENTADAFDGDLGMGAPDAKARDEAYVMAGVADIFHKNNQLAVKVLGSGAFATLQGLGISWAIAFGPEADQCVPDLKLSYMPIWKFADLKPLQRLRIVRAIISAVVGILMELAALCRWATGVVKDVFAFADLVKIPVAIKVSIISAVVGIPISAVVGILLPILQPEQLQVFADGKYSEDPTVPLADMQIAKGMSYARGAPPSTFKADLQVIRGRILPDGRASPLTSIIADLVHRDLAARADSWLGLRSLRADGKLGISWLGLRADGVKITDFGLARATDFGLARLLPDGDSTFYRSLLAILLVVVLGVADTTPVTGASPRDLRIVRGTQLATELVEPLTPPDLKASCVTACPYPILAALCRWGLADAFEDNYALAVAIDVWSYGVTVAWGLLLALLPRDAKQLMPYGCLLAIKIFGSLAFLALCRWGLLLRDGRIVRGTQLFADLVGSGAFGTVYADGGSCTLVCPLPDGYISAWPDSLRDLHCPALVTYALLVCRWGLLLALRWGLLLALL

SEQ ID NO: 93 - protein, 461 aa, Artificial Sequence
  source: /note=“Description of Artificial Sequence Synthetic polypeptide”
MELAALCRWGLLLALLPPGAPDGENVKIPVAIKVLRENTADGKEECRVLQGLPDGKYSEDPTVPLPDDEAYVMAGVADLKQETELVEPLTPPDGRASPLTSIISAVVGILLVVVLGVVFPDAGMEHLREVRADGKDIFHKNNQLPDLQPEQLQVFRDAQEVQGYVLIPDLAFDGDLGMGAPDLQVIRGRILPDVKVLGSGAFGTVYPIGDLTLGLEPPDLKASCVTACPYATLQGLGISWLGLRSLRELGSGLALPMQIAKGMSYALFGPEADQCVPDLKLSYMPIWKFADLKPLQRLRIVRGTQLFEDNYALAVARGAPPSTFKAGVVKDVFAFRDLVKITDFGLARLLPLVHRDLAARADVWSYGVTVRDTTPVTGASPRDLYISAWPDSLRTVCAGGCARSDKIFGSLAFLPDLHCPALVTYADDSTFYRSLLADGKQLMPYGCLLADGGSCTLVCPL

SEQ ID NO: 94 - protein, 9 aa, Artificial Sequence
  source: /note=“Description of Artificial Sequence Synthetic peptide”
LTCSPQPEY

SEQ ID NO: 95 - protein, 9 aa, Artificial Sequence
  source: /note=“Description of Artificial Sequence Synthetic peptide”
EGAGSDVFD

SEQ ID NO: 96 - protein, 9 aa, Artificial Sequence
  source: /note=“Description of Artificial Sequence Synthetic peptide”
YKDPPFCVA

SEQ ID NO: 97 - protein, 9 aa, Artificial Sequence
  source: /note=“Description of Artificial Sequence Synthetic peptide”
TIDVYMIMV

SEQ ID NO: 98 - protein, 9 aa, Artificial Sequence
  source: /note=“Description of Artificial Sequence Synthetic peptide”
YGVTVWELM

SEQ ID NO: 99 - protein, 9 aa, Artificial Sequence
  source: /note=“Description of Artificial Sequence Synthetic peptide”
LLDIDETEY

SEQ ID NO: 100 - protein, 9 aa, Artificial Sequence
  source: /note=“Description of Artificial Sequence Synthetic peptide”
QSDVWSYGV

SEQ ID NO: 101 - protein, 9 aa, Artificial Sequence
  source: /note=“Description of Artificial Sequence Synthetic peptide”
HLDMLRHLY

SEQ ID NO: 102 - protein, 9 aa, Artificial Sequence
  source: /note=“Description of Artificial Sequence Synthetic peptide”
DGDPASNTA

SEQ ID NO: 103 - protein, 9 aa, Artificial Sequence
  source: /note=“Description of Artificial Sequence Synthetic peptide”
NASLSFLQD

SEQ ID NO: 104 - protein, 9 aa, Artificial Sequence
  source: /note=“Description of Artificial Sequence Synthetic peptide”
DGDLGMGAA

SEQ ID NO: 105 - protein, 9 aa, Artificial Sequence
  source: /note=“Description of Artificial Sequence Synthetic peptide”
FSPAFDNLY

SEQ ID NO: 106 - protein, 9 aa, Artificial Sequence
  source: /note=“Description of Artificial Sequence Synthetic peptide”
EITGYLYIS

SEQ ID NO: 107 - protein, 9 aa, Artificial Sequence
  source: /note=“Description of Artificial Sequence Synthetic peptide”
DDDMGDLVD

SEQ ID NO: 108 - protein, 9 aa, Artificial Sequence
  source: /note=“Description of Artificial Sequence Synthetic peptide”
HSDCLACLH

SEQ ID NO: 109 - protein, 9 aa, Artificial Sequence
  source: /note=“Description of Artificial Sequence Synthetic peptide”
DIDETEYHA

SEQ ID NO: 110 - protein, 275 aa, Artificial Sequence
  source: /note=“Description of Artificial Sequence Synthetic polypeptide”
WGLLLALLPRDAYSEDPTVPLADIDETEYHAPDLKAREEGAGSDVFDAYGVTVWELMALGKARDDDDMGDLVDPLGKAEITGYLYISADGKHLDMLRHLYADLKAHSDCLACLHADLTCSPQPEYADLKQSDVWSYGVADAYKDPPFCVAPDLARDGDLGMGAAPIAKLLDIDETEYADARDGDPASNTAAIARDGENVKIPVALLGSGAFGTVYPDNASLSFLQDPLLKLHCPALVTYADDSTFYRSLLADLFSPAFDNLYAILKTIDVYMIMV

SEQ ID NO: 111 - protein, 9 aa, Artificial Sequence
  source: /note=“Description of Artificial Sequence Synthetic peptide”
LLLALLPPG

SEQ ID NO: 112 - protein, 9 aa, Artificial Sequence
  source: /note=“Description of Artificial Sequence Synthetic peptide”
ILDEAYVMA

SEQ ID NO: 113 - protein, 9 aa, Artificial Sequence
  source: /note=“Description of Artificial Sequence Synthetic peptide”
ILHNGAYSL

SEQ ID NO: 114 - protein, 9 aa, Artificial Sequence
  source: /note=“Description of Artificial Sequence Synthetic peptide”
RLLQETELV

SEQ ID NO: 115 - protein, 9 aa, Artificial Sequence
  source: /note=“Description of Artificial Sequence Synthetic peptide”
MIMVKCWMI

SEQ ID NO: 116 - protein, 9 aa, Artificial Sequence
  source: /note=“Description of Artificial Sequence Synthetic peptide”
LVDAEEYLV

SEQ ID NO: 117 - protein, 9 aa, Artificial Sequence
  source: /note=“Description of Artificial Sequence Synthetic peptide”
RLVHRDLAA

SEQ ID NO: 118 - protein, 9 aa, Artificial Sequence
  source: /note=“Description of Artificial Sequence Synthetic peptide”
LLNWCMQIA

SEQ ID NO: 119 - protein, 9 aa, Artificial Sequence
  source: /note=“Description of Artificial Sequence Synthetic peptide”
ALIHHNTHL

SEQ ID NO: 120 - protein, 9 aa, Artificial Sequence
  source: /note=“Description of Artificial Sequence Synthetic peptide”
AVVGILLVV

SEQ ID NO: 121 - protein, 9 aa, Artificial Sequence
  source: /note=“Description of Artificial Sequence Synthetic peptide”
VVLGVVFGI

SEQ ID NO: 122 - protein, 9 aa, Artificial Sequence
  source: /note=“Description of Artificial Sequence Synthetic peptide”
SLTLQGLGI

SEQ ID NO: 123 - protein, 252 aa, Artificial Sequence
  source: /note=“Description of Artificial Sequence Synthetic polypeptide”
TIDVYMIMVPDLKCRWGLLLALALLALLPPGAADGAILDEAYVMAALIHHNTHLPDLRLVHRDLAALLLALLPPGADGKQLFEDNYALPILHNGAYSLPSLTLQGLGIRLVDAEEYLVRILLVVVLGVADASIISAVVGIARLLQETELVADAFEDNYALAVAVVGILLVVAVVLGVVFGIADALLNWCMQIAADLVALCRWGLLLADYISAWPDSLRDKIFGSLAFLRDLQLMPYGCLLADGMIMVKCWMI

SEQ ID NO: 124 - protein, 168 aa, Artificial Sequence
  source: /note=“Description of Artificial Sequence Synthetic polypeptide”
MELAALCRWGLLLALLPPGAPPDLLALLPPGAPDATLEEITGYLAILDEAYVMAPILHNGAYSLPQLFEDNYALSIISAVVGIAQLMPYGCLLRLLVVVLGVVRDLQLRSLTEIAILLVVVLGVPDAVVGILLVVADALCRWGLLLADYISAWPDSLRDKIFGSLAFL

SEQ ID NO: 125 - protein, 9 aa, Artificial Sequence
  source: /note=“Description of Artificial Sequence Synthetic peptide”
CLTSTVQLV

SEQ ID NO: 126 - protein, 9 aa, Artificial Sequence
  source: /note=“Description of Artificial Sequence Synthetic peptide”
QIAKGMSYL

SEQ ID NO: 127 - protein, 9 aa, Artificial Sequence
  source: /note=“Description of Artificial Sequence Synthetic peptide”
PCARVCYGL

SEQ ID NO: 128 - protein, 9 aa, Artificial Sequence
  source: /note=“Description of Artificial Sequence Synthetic peptide”
KHSDCLACL

SEQ ID NO: 129 - protein, 9 aa, Artificial Sequence
  source: /note=“Description of Artificial Sequence Synthetic peptide”
TYLPTNASL

SEQ ID NO: 130 - protein, 9 aa, Artificial Sequence
  source: /note=“Description of Artificial Sequence Synthetic peptide”
SLPDLSVFQ

SEQ ID NO: 131 - protein, 9 aa, Artificial Sequence
  source: /note=“Description of Artificial Sequence Synthetic peptide”
FRNPHQALL

SEQ ID NO: 132 - protein, 9 aa, Artificial Sequence
  source: /note=“Description of Artificial Sequence Synthetic peptide”
TLEEITGYL

SEQ ID NO: 133 - protein, 9 aa, Artificial Sequence
  source: /note=“Description of Artificial Sequence Synthetic peptide”
DLVDAEEYL

SEQ ID NO: 134 - protein, 9 aa, Artificial Sequence
  source: /note=“Description of Artificial Sequence Synthetic peptide”
AYSLTLQGL

SEQ ID NO: 135 - protein, 9 aa, Artificial Sequence
  source: /note=“Description of Artificial Sequence Synthetic peptide”
LVPQQGFFC

SEQ ID NO: 136 - protein, 9 aa, Artificial Sequence
  source: /note=“Description of Artificial Sequence Synthetic peptide”
THLDMLRHL

SEQ ID NO: 137 - protein, 9 aa, Artificial Sequence
  source: /note=“Description of Artificial Sequence Synthetic peptide”
TLSPGKNGV

SEQ ID NO: 138 - protein, 286 aa, Artificial Sequence
  source: /note=“Description of Artificial Sequence Synthetic polypeptide”
LVPQQGFFCADLVPCARVCYGLPDLKKHSDCLACLATLEEITGYLATLSPGKNGVPDLDLVDAEEYLPILHNGAYSLASLPDLSVFQRDQIAKGMSYLAILDEAYVMAALIHHNTHLAIAFGPEADQCVRDLKLVDAEEYLVAQLFEDNYALSIISAVVGIADGTHLDMLRHLACLTSTVQLVADGFRNPHQALLADGRLLQETELVADLKIFGSLAFLAYISAWPDSLRDAYSLTLQGLRDLTYLPTNASLSDARWGLLLALLAQLMPYGCLLADGMIMVKCWMI

SEQ ID NO: 139 - protein, 9 aa, Artificial Sequence
  source: /note=“Description of Artificial Sequence Synthetic peptide”
HYKDPPFCV

SEQ ID NO: 140 - protein, 9 aa, Artificial Sequence
  source: /note=“Description of Artificial Sequence Synthetic peptide”
YLTPQGGAA

SEQ ID NO: 141 - protein, 9 aa, Artificial Sequence
  source: /note=“Description of Artificial Sequence Synthetic peptide”
SLRELGSGL

SEQ ID NO: 142 - protein, 9 aa, Artificial Sequence
  source: /note=“Description of Artificial Sequence Synthetic peptide”
HLYQGCQVV

SEQ ID NO: 143 - protein, 9 aa, Artificial Sequence
  source: /note=“Description of Artificial Sequence Synthetic peptide”
PLTSIISAV

SEQ ID NO: 144 - protein, 9 aa, Artificial Sequence
  source: /note=“Description of Artificial Sequence Synthetic peptide”
PYVSRLLGI

SEQ ID NO: 145 - protein, 9 aa, Artificial Sequence
  source: /note=“Description of Artificial Sequence Synthetic peptide”
IQNEDLGPA

SEQ ID NO: 146 - protein, 9 aa, Artificial Sequence
  source: /note=“Description of Artificial Sequence Synthetic peptide”
YLSTDVGSC

SEQ ID NO: 147 - protein, 9 aa, Artificial Sequence
  source: /note=“Description of Artificial Sequence Synthetic peptide”
LLVVVLGVV

SEQ ID NO: 148 - protein, 275 aa, Artificial Sequence
  source: /note=“Description of Artificial Sequence Synthetic polypeptide”
HYKDPPFCVAIGKAIQNEDLGPARDLQIAKGMSYLATLSPGKNGVSDLLALLPPGAADGPYVSRLLGIAYLSTDVGSCADILLVVVLGVADASIISAVVGIADSLRELGSGLPTGRASPLTSIIALLVVVLGVVRDLAYLTPQGGAAALIHHNTHLADARPLTSIISAVADLFRNPHQALLADGKKIFGSLAFLALLNWCMQIAADLKACLTSTVQLVADGYISAWPDSLAHLYQGCQVVADLSLTLQGLGIADQLMPYGCLLADGMIMVKCWMI

SEQ ID NO: 149 - protein, 9 aa, Artificial Sequence
  source: /note=“Description of Artificial Sequence Synthetic peptide”
QVFETLEEI

SEQ ID NO: 150 - protein, 9 aa, Artificial Sequence
  source: /note=“Description of Artificial Sequence Synthetic peptide”
LQLRSLTEI

SEQ ID NO: 151 - protein, 9 aa, Artificial Sequence
  source: /note=“Description of Artificial Sequence Synthetic peptide”
YVLIAHNQV

SEQ ID NO: 152 - protein, 9 aa, Artificial Sequence
  source: /note=“Description of Artificial Sequence Synthetic peptide”
AVLDNGDPL

SEQ ID NO: 153 - protein, 9 aa, Artificial Sequence
  source: /note=“Description of Artificial Sequence Synthetic peptide”
IWIPDGENV

SEQ ID NO: 154 - protein, 9 aa, Artificial Sequence
  source: /note=“Description of Artificial Sequence Synthetic peptide”
TQLFEDNYA

SEQ ID NO: 155 - protein, 9 aa, Artificial Sequence
  source: /note=“Description of Artificial Sequence Synthetic peptide”
SAVVGILLV

SEQ ID NO: 156 - protein, 269 aa, Artificial Sequence
  source: /note=“Description of Artificial Sequence Synthetic polypeptide”
CRWGLLLALPDAIQNEDLGPAAVLDNGDPLRLLQETELVADGFRNPHQALLPDLKQVFETLEEIPDQIAKGMSYLPDVVLGVVFGIADATQLFEDNYAADAVVGILLVVADRASPLTSIIALLVVVLGVVRDLQLRSLTEIAILLVVVLGVADASIISAVVGIPDYVLIAHNQVADVKIPVAIKVALIHHNTHLALAALCRWGLASAVVGILLVADGKKIFGSLAFLAIWIPDGENVADTIDVYMIMVQLMPYGCLLADGMIMVKCWMI

SEQ ID NO: 157 - protein, 9 aa, Artificial Sequence
  source: /note=“Description of Artificial Sequence Synthetic peptide”
LAARNVLVK

SEQ ID NO: 158 - protein, 9 aa, Artificial Sequence
  source: /note=“Description of Artificial Sequence Synthetic peptide”
VVFGILIKR

SEQ ID NO: 159 - protein, 9 aa, Artificial Sequence
  source: /note=“Description of Artificial Sequence Synthetic peptide”
VMAGVGSPY

SEQ ID NO: 160 - protein, 9 aa, Artificial Sequence
  source: /note=“Description of Artificial Sequence Synthetic peptide”
RILHNGAYS

SEQ ID NO: 161 - protein, 9 aa, Artificial Sequence
  source: /note=“Description of Artificial Sequence Synthetic peptide”
TFYRSLLED

SEQ ID NO: 162 - protein, 9 aa, Artificial Sequence
  source: /note=“Description of Artificial Sequence Synthetic peptide”
VVVLGVVFG

SEQ ID NO: 163 - protein, 9 aa, Artificial Sequence
  source: /note=“Description of Artificial Sequence Synthetic peptide”
QLVTQLMPY

SEQ ID NO: 164 - protein, 9 aa, Artificial Sequence
  source: /note=“Description of Artificial Sequence Synthetic peptide”
GILLVVVLG

SEQ ID NO: 165 - protein, 9 aa, Artificial Sequence
  source: /note=“Description of Artificial Sequence Synthetic peptide”
LELTYLPTN

SEQ ID NO: 166 - protein, 9 aa, Artificial Sequence
  source: /note=“Description of Artificial Sequence Synthetic peptide”
LVKSPNHVK

SEQ ID NO: 167 - protein, 9 aa, Artificial Sequence
  source: /note=“Description of Artificial Sequence Synthetic peptide”
ELMTFGAKP

SEQ ID NO: 168 - protein, 9 aa, Artificial Sequence
  source: /note=“Description of Artificial Sequence Synthetic peptide”
TVWELMTFG

SEQ ID NO: 169 - protein, 9 aa, Artificial Sequence
  source: /note=“Description of Artificial Sequence Synthetic peptide”
YLYISAWPD

SEQ ID NO: 170 - protein, 9 aa, Artificial Sequence
  source: /note=“Description of Artificial Sequence Synthetic peptide”
ILKETELRK

SEQ ID NO: 171 - protein, 9 aa, Artificial Sequence
  source: /note=“Description of Artificial Sequence Synthetic peptide”
YTMRRLLQE

SEQ ID NO: 172 - protein, 9 aa, Artificial Sequence
  source: /note=“Description of Artificial Sequence Synthetic peptide”
RSLTEILKG

SEQ ID NO: 173 - protein, 9 aa, Artificial Sequence
  source: /note=“Description of Artificial Sequence Synthetic peptide”
GVVFGILIK

SEQ ID NO: 174 - protein, 9 aa, Artificial Sequence
  source: /note=“Description of Artificial Sequence Synthetic peptide”
VLRENTSPK

SEQ ID NO: 175 - protein, 9 aa, Artificial Sequence
  source: /note=“Description of Artificial Sequence Synthetic peptide”
CVNCSQFLR

SEQ ID NO: 176 - protein, 9 aa, Artificial Sequence
  source: /note=“Description of Artificial Sequence Synthetic peptide”
ILIKRRQQK

SEQ ID NO: 177 - protein, 9 aa, Artificial Sequence
  source: /note=“Description of Artificial Sequence Synthetic peptide”
LERPKTLSP

SEQ ID NO: 178 - protein, 9 aa, Artificial Sequence
  source: /note=“Description of Artificial Sequence Synthetic peptide”
MTFGAKPYD

SEQ ID NO: 179 - protein, 9 aa, Artificial Sequence
  source: /note=“Description of Artificial Sequence Synthetic peptide”
ALLHTANRP

SEQ ID NO: 180 - protein, 9 aa, Artificial Sequence
  source: /note=“Description of Artificial Sequence Synthetic peptide”
KIRKYTMRR

SEQ ID NO: 181 - protein, 9 aa, Artificial Sequence
  source: /note=“Description of Artificial Sequence Synthetic peptide”
ILWKDIFHK

SEQ ID NO: 182 - protein, 9 aa, Artificial Sequence
  source: /note=“Description of Artificial Sequence Synthetic peptide”
LAFLPESFD

SEQ ID NO: 183 - protein, 322 aa, Artificial Sequence
  source: /note=“Description of Artificial Sequence Synthetic polypeptide”
CVNCSQFLRADLVKSPNHVKAILKETELRKRDLKARILHNGAYSADGVVFGILIKADGAELMTFGAKPPDGKLELTYLPTNALGKKIRKYTMRRADLVLERPKTLSPAVLRENTSPKALLLALLPPGADGKRSLTEILKGALLHTANRPAILIKRRQQKADGKAGILLVVVLGPDGKTVWELMTFGAILWKDIFHKADGKRGAPPSTFKADLQLVTQLMPYAVVVLGVVFGPDVMAGVGSPYAILKLAARNVLVKADLYTMRRLLQEADGKTFYRSLLEDRDVVFGILIKRALAFLPESFDAYLYISAWPDADMTFGAKPYD

SEQ ID NO: 184 - protein, 9 aa, Artificial Sequence
  source: /note=“Description of Artificial Sequence Synthetic peptide”
VYMIMVKCW

SEQ ID NO: 185 - protein, 9 aa, Artificial Sequence
  source: /note=“Description of Artificial Sequence Synthetic peptide”
SYGVTVWEL

SEQ ID NO: 186 - protein, 9 aa, Artificial Sequence
  source: /note=“Description of Artificial Sequence Synthetic peptide”
EYLVPQQGF

SEQ ID NO: 187 - protein, 9 aa, Artificial Sequence
  source: /note=“Description of Artificial Sequence Synthetic peptide”
EYHADGGKV

SEQ ID NO: 188 - protein, 9 aa, Artificial Sequence
  source: /note=“Description of Artificial Sequence Synthetic peptide”
RFRELVSEF

SEQ ID NO: 189 - protein, 9 aa, Artificial Sequence
  source: /note=“Description of Artificial Sequence Synthetic peptide”
EYVNARHCL

SEQ ID NO: 190 - protein, 9 aa, Artificial Sequence
  source: /note=“Description of Artificial Sequence Synthetic peptide”
KWMALESIL

SEQ ID NO: 191 - protein, 9 aa, Artificial Sequence
  source: /note=“Description of Artificial Sequence Synthetic peptide”
DLLEKGERL

SEQ ID NO: 192 - protein, 9 aa, Artificial Sequence
  source: /note=“Description of Artificial Sequence Synthetic peptide”
VWSYGVTVW

SEQ ID NO: 193 - protein, 9 aa, Artificial Sequence
  source: /note=“Description of Artificial Sequence Synthetic peptide”
QCVNCSQFL

SEQ ID NO: 194 - protein, 246 aa, Artificial Sequence
  source: /note=“Description of Artificial Sequence Synthetic polypeptide”
RWGLLLALLAEYVNARHCLRDLLEKGERLAEYHADGGKVSDIFHKNNQLAQLFEDNYALPLAALCRWGLAIAYGVTVWELMAILRIVRGTQLILLVVVLGVADATYLPTNASLAIWIPDGENVRLLVWSYGVTVWALEYLVPQQGFADLKDVWSYGVTVPDLKRFRELVSEFPDLKLSYMPIWKFADLSYGVTVWELADAQCVNCSQFLADAKVYMIMVKCWAILKKWMALESILAIMIMVKCWMI

SEQ ID NO: 195 - protein, 9 aa, Artificial Sequence
  source: /note=“Description of Artificial Sequence Synthetic peptide”
SLAFLPESF

SEQ ID NO: 196 - protein, 9 aa, Artificial Sequence
  source: /note=“Description of Artificial Sequence Synthetic peptide”
AWPDSLPDL

SEQ ID NO: 197 - protein, 187 aa, Artificial Sequence
  source: /note=“Description of Artificial Sequence Synthetic polypeptide”
AWPDSLPDLDLLEKGERLRDGPYVSRLLGIPDLTLQGLGISWASLAFLPESFPDGKAVVGILLVVRTLVVVLGVVFAIWIPDGENVRLLVWSYGVTVWALEYLVPQQGFADLKQLMPYGCLLADSYGVTVWELADLTYLPTNASLARIVRGTQLFRWGLLLALLAKWMALESILAIGVVYMIMVKCW

SEQ ID NO: 198 - protein, 9 aa, Artificial Sequence
  source: /note=“Description of Artificial Sequence Synthetic peptide”
FYRSLLEDD

SEQ ID NO: 199 - protein, 9 aa, Artificial Sequence
  source: /note=“Description of Artificial Sequence Synthetic peptide”
CYGLGMEHL

SEQ ID NO: 200 - protein, 9 aa, Artificial Sequence
  source: /note=“Description of Artificial Sequence Synthetic peptide”
LQGLGISWL

SEQ ID NO: 201 - protein, 9 aa, Artificial Sequence
  source: /note=“Description of Artificial Sequence Synthetic peptide”
SEGAGSDVF

SEQ ID NO: 202 - protein, 9 aa, Artificial Sequence
  source: /note=“Description of Artificial Sequence Synthetic peptide”
RMARDPQRF

SEQ ID NO: 203 - protein, 9 aa, Artificial Sequence
  source: /note=“Description of Artificial Sequence Synthetic peptide”
VTVWELMTF

SEQ ID NO: 204 - protein, 9 aa, Artificial Sequence
  source: /note=“Description of Artificial Sequence Synthetic peptide”
YLVPQQGFF

SEQ ID NO: 205 - protein, 9 aa, Artificial Sequence
  source: /note=“Description of Artificial Sequence Synthetic peptide”
HNGAYSLTL

SEQ ID NO: 206 - protein, 9 aa, Artificial Sequence
  source: /note=“Description of Artificial Sequence Synthetic peptide”
GEGLACHQL

SEQ ID NO: 207 - protein, 9 aa, Artificial Sequence
  source: /note=“Description of Artificial Sequence Synthetic peptide”
FQNLQVIRG

SEQ ID NO: 208 - protein, 9 aa, Artificial Sequence
  source: /note=“Description of Artificial Sequence Synthetic peptide”
RFTHQSDVW

SEQ ID NO: 209 - protein, 9 aa, Artificial Sequence
  source: /note=“Description of Artificial Sequence Synthetic peptide”
FNHSGICEL

SEQ ID NO: 210 - protein, 9 aa, Artificial Sequence
  source: /note=“Description of Artificial Sequence Synthetic peptide”
VRGTQLFED

SEQ ID NO: 211 - protein, 291 aa, Artificial Sequence
  source: /note=“Description of Artificial Sequence Synthetic polypeptide”
RMARDPQRFADAVRGTQLFEDRDLQPEQLQVFADGEYVNARHCLADARWGLLLALLASEGAGSDVFAGEGLACHQLPDLKLQGLGISWLAISYGVTVWELADAWPDSLPDLPLEYLVPQQGFADGKHNGAYSLTLAFNHSGICELAYLVPQQGFFADGVAYSLTLQGLPDLKRFRELVSEFADGKACYGLGMEHLALVWSYGVTVWAIAFQNLQVIRGADGVTVWELMTFADGKAFYRSLLEDDRDLTYLPTNASLAIVYMIMVKCWAILKKWMALESILADRFTHQSDVW

SEQ ID NO: 212 - protein, 9 aa, Artificial Sequence
  source: /note=“Description of Artificial Sequence Synthetic peptide”
ISWLGLRSL

SEQ ID NO: 213 - protein, 9 aa, Artificial Sequence
  source: /note=“Description of Artificial Sequence Synthetic peptide”
ETLEEITGY

SEQ ID NO: 214 - protein, 9 aa, Artificial Sequence
  source: /note=“Description of Artificial Sequence Synthetic peptide”
DTILWKDIF

SEQ ID NO: 215 - protein, 9 aa, Artificial Sequence
  source: /note=“Description of Artificial Sequence Synthetic peptide”
HTVPWDQLF

SEQ ID NO: 216 - protein, 9 aa, Artificial Sequence
  source: /note=“Description of Artificial Sequence Synthetic peptide”
ICELHCPAL

SEQ ID NO: 217 - protein, 9 aa, Artificial Sequence
  source: /note=“Description of Artificial Sequence Synthetic peptide”
VSRLLGICL

SEQ ID NO: 218 - protein, 9 aa, Artificial Sequence
  source: /note=“Description of Artificial Sequence Synthetic peptide”
DVFDGDLGM

SEQ ID NO: 219 - protein, 9 aa, Artificial Sequence
  source: /note=“Description of Artificial Sequence Synthetic peptide”
ETHLDMLRH

SEQ ID NO: 220 - protein, 9 aa, Artificial Sequence
  source: /note=“Description of Artificial Sequence Synthetic peptide”
KVPIKWMAL

SEQ ID NO: 221 - protein, 9 aa, Artificial Sequence
  source: /note=“Description of Artificial Sequence Synthetic peptide”
CTIDVYMIM

SEQ ID NO: 222 - protein, 9 aa, Artificial Sequence
  source: /note=“Description of Artificial Sequence Synthetic peptide”
TEILKGGVL

SEQ ID NO: 223 - protein, 9 aa, Artificial Sequence
  source: /note=“Description of Artificial Sequence Synthetic peptide”
STVQLVTQL

SEQ ID NO: 224 - protein, 257 aa, Artificial Sequence
  source: /note=“Description of Artificial Sequence Synthetic polypeptide”
CTIDVYMIMPIICELHCPALAQLVTQLMPYADGVSRLLGICLALCRWGLLLPDLKARDEAYVMAGVADETLEEITGYATEILKGGVLPQLFEDNYALPDLQPEQLQVFADKVPIKWMALSIISAVVGIRDDTILWKDIFALGVAETHLDMLRHADVFDGDLGMPDLKSLRELGSGLSTVQLVTQLPLGKISWLGLRSLAFDGDLGMGAADCRWGLLLALPDVTVWELMTFADGKAFEDNYALAVRDLKHTVPWDQLF

SEQ ID NO: 225 - protein, 9 aa, Artificial Sequence
  source: /note=“Description of Artificial Sequence Synthetic peptide”
ICLTSTVQL

SEQ ID NO: 226 - protein, 9 aa, Artificial Sequence
  source: /note=“Description of Artificial Sequence Synthetic peptide”
YLEDVRLVH

SEQ ID NO: 227 - protein, 9 aa, Artificial Sequence
  source: /note=“Description of Artificial Sequence Synthetic peptide”
THQSDVWSY

SEQ ID NO: 228 - protein, 9 aa, Artificial Sequence
  source: /note=“Description of Artificial Sequence Synthetic peptide”
GTVYKGIWI

SEQ ID NO: 229 - protein, 9 aa, Artificial Sequence
  source: /note=“Description of Artificial Sequence Synthetic peptide”
SMPNPEGRY

SEQ ID NO: 230 - protein, 9 aa, Artificial Sequence
  source: /note=“Description of Artificial Sequence Synthetic peptide”
GTQLFEDNY

SEQ ID NO: 231 - protein, 9 aa, Artificial Sequence
  source: /note=“Description of Artificial Sequence Synthetic peptide”
VCTGTDMKL

SEQ ID NO: 232 - protein, 9 aa, Artificial Sequence
  source: /note=“Description of Artificial Sequence Synthetic peptide”
LEEITGYLY

SEQ ID NO: 233 - protein, 9 aa, Artificial Sequence
  source: /note=“Description of Artificial Sequence Synthetic peptide”
LMTFGAKPY

SEQ ID NO: 234 - protein, 9 aa, Artificial Sequence
  source: /note=“Description of Artificial Sequence Synthetic peptide”
QVVQGNLEL

SEQ ID NO: 235 - protein, 9 aa, Artificial Sequence
  source: /note=“Description of Artificial Sequence Synthetic peptide”
SPAFDNLYY

SEQ ID NO: 236 - protein, 9 aa, Artificial Sequence
  source: /note=“Description of Artificial Sequence Synthetic peptide”
PGGLRELQL

SEQ ID NO: 237 - protein, 9 aa, Artificial Sequence
  source: /note=“Description of Artificial Sequence Synthetic peptide”
CVTACPYNY

SEQ ID NO: 238 - protein, 9 aa, Artificial Sequence
  source: /note=“Description of Artificial Sequence Synthetic peptide”
MSYLEDVRL

SEQ ID NO: 239 - protein, 339 aa, Artificial Sequence
  source: /note=“Description of Artificial Sequence Synthetic polypeptide”
LHCPALVTYSDLTCSPQPEYADLRLVHRDLAAALGHLDMLRHLYADLVVVLGVVFPDGKDIFHKNNQLADLEEITGYLYADGVVKDVFAFADARPGGLRELQLADETLEEITGYALLTHQSDVWSYADAYLEDVRLVHPDLKQVVQGNLELAIGSGAFGTVYRLVMAGVGSPYAILKLMTFGAKPYADGTQLFEDNYADGKCVTACPYNYADGGTVYKGIWIADLSMPNPEGRYADLKHTVPWDQLFADLKSLTLQGLGIADMQIAKGMSYAICLTSTVQLSDDVWSYGVTVPDLKMSYLEDVRLRDVCTGTDMKLADFSPAFDNLYAILSPAFDNLYY

SEQ ID NO: 240 - protein, 9 aa, Artificial Sequence
  source: /note=“Description of Artificial Sequence Synthetic peptide”
GGKVPIKWM

SEQ ID NO: 241 - protein, 9 aa, Artificial Sequence
  source: /note=“Description of Artificial Sequence Synthetic peptide”
RSRACHPCS

SEQ ID NO: 242 - protein, 9 aa, Artificial Sequence
  source: /note=“Description of Artificial Sequence Synthetic peptide”
HVRENRGRL

SEQ ID NO: 243 - protein, 9 aa, Artificial Sequence
  source: /note=“Description of Artificial Sequence Synthetic peptide”
MARDPQRFV

SEQ ID NO: 244 - protein, 9 aa, Artificial Sequence
  source: /note=“Description of Artificial Sequence Synthetic peptide”
AARNVLVKS

SEQ ID NO: 245 - protein, 9 aa, Artificial Sequence
  source: /note=“Description of Artificial Sequence Synthetic peptide”
TQRCEKCSK

SEQ ID NO: 246 - protein, 9 aa, Artificial Sequence
  source: /note=“Description of Artificial Sequence Synthetic peptide”
LQRLRIVRG

SEQ ID NO: 247 - protein, 9 aa, Artificial Sequence
  source: /note=“Description of Artificial Sequence Synthetic peptide”
STFKGTPTA

SEQ ID NO: 248 - protein, 9 aa, Artificial Sequence
  source: /note=“Description of Artificial Sequence Synthetic peptide”
KLRLPASPE

SEQ ID NO: 249 - protein, 9 aa, Artificial Sequence
  source: /note=“Description of Artificial Sequence Synthetic peptide”
KRRQQKIRK

SEQ ID NO: 250 - protein, 9 aa, Artificial Sequence
  source: /note=“Description of Artificial Sequence Synthetic peptide”
KETELRKVK

SEQ ID NO: 251 - protein, 9 aa, Artificial Sequence
  source: /note=“Description of Artificial Sequence Synthetic peptide”
NVKIPVAIK

SEQ ID NO: 252 - protein, 9 aa, Artificial Sequence
  source: /note=“Description of Artificial Sequence Synthetic peptide”
RPKTLSPGK

SEQ ID NO: 253 - protein, 9 aa, Artificial Sequence
  source: /note=“Description of Artificial Sequence Synthetic peptide”
VARCPSGVK

SEQ ID NO: 254 - protein, 9 aa, Artificial Sequence
  source: /note=“Description of Artificial Sequence Synthetic peptide”
GCLLDHVRE

SEQ ID NO: 255 - protein, 9 aa, Artificial Sequence
  source: /note=“Description of Artificial Sequence Synthetic peptide”
PGKNGVVKD

SEQ ID NO: 256 - protein, 9 aa, Artificial Sequence
  source: /note=“Description of Artificial Sequence Synthetic peptide”
GLRSLRELG

SEQ ID NO: 257 - protein, 9 aa, Artificial Sequence
  source: /note=“Description of Artificial Sequence Synthetic peptide”
KVKVLGSGA

SEQ ID NO: 258 - protein, 308 aa, Artificial Sequence
  source: /note=“Description of Artificial Sequence Synthetic polypeptide”
KIRKYTMRRAYLYISAWPDLVKSPNHVKPLLKKVKVLGSGAPDGKETELRKVKPDAIKVLRENTADGGKVPIKWMADGNVKIPVAIKADARGGCLLDHVREAGLRSLRELGADGRPKTLSPGKAILQRLRIVRGPDGVKLRLPASPEAWGLLLALLPADRSRACHPCSAILKKRRQQKIRKADLKHVRENRGRLADARPGKNGVVKDAPLQRLRIVRRDAKAARNVLVKSADMARDPQRFVAVLRENTSPKADLVARCPSGVKADLHYKDPPFCVADKIFGSLAFLASTFKGTPTAADLTQRCEKCSK

SEQ ID NO: 259 - protein, 9 aa, Artificial Sequence
  source: /note=“Description of Artificial Sequence Synthetic peptide”
ESFDGDPAS

SEQ ID NO: 260 - protein, 9 aa, Artificial Sequence
  source: /note=“Description of Artificial Sequence Synthetic peptide”
KGMSYLEDV

SEQ ID NO: 261 - protein, 9 aa, Artificial Sequence
  source: /note=“Description of Artificial Sequence Synthetic peptide”
VVKDVFAFG

SEQ ID NO: 262 - protein, 9 aa, Artificial Sequence
  source: /note=“Description of Artificial Sequence Synthetic peptide”
DMGDLVDAE

SEQ ID NO: 263 - protein, 9 aa, Artificial Sequence
  source: /note=“Description of Artificial Sequence Synthetic peptide”
GGAVENPEY

SEQ ID NO: 264 - protein, 9 aa, Artificial Sequence
  source: /note=“Description of Artificial Sequence Synthetic peptide”
SLTEILKGG

SEQ ID NO: 265 - protein, 9 aa, Artificial Sequence
  source: /note=“Description of Artificial Sequence Synthetic peptide”
RSSSTRSGG

SEQ ID NO: 266 - protein, 9 aa, Artificial Sequence
  source: /note=“Description of Artificial Sequence Synthetic peptide”
VLQGLPREY

SEQ ID NO: 267 - protein, 9 aa, Artificial Sequence
  source: /note=“Description of Artificial Sequence Synthetic peptide”
YYWDQDPPE

SEQ ID NO: 268 - protein, 9 aa, Artificial Sequence
  source: /note=“Description of Artificial Sequence Synthetic peptide”
EIPDLLEKG

SEQ ID NO: 269 - protein, 9 aa, Artificial Sequence
  source: /note=“Description of Artificial Sequence Synthetic peptide”
APRSPLAPS

SEQ ID NO: 270 - protein, 349 aa, Artificial Sequence
  source: /note=“Description of Artificial Sequence Synthetic polypeptide”
SMPNPEGRYADLKHSDCLACLADMGDLVDAERDGKCVTACPYNYALGGAVENPEYALAVVKDVFAFGPLAKAEIPDLLEKGPDGKHLDMLRHLYADLKTVWELMTFGADLTCSPQPEYADLRSSSTRSGGADGKETLEEITGYADVLQGLPREYADARPLTSIISAVALASCVTACPYPLLSAVVGILLVADLVAESFDGDPASRDVFDGDLGMPILAAPRSPLAPSAIGTQLFEDNYAIGASLTEILKGGADKGMSYLEDVADVMAGVGSPYATLKSLPDLSVFQRDLKTHQSDVWSYADASPAFDNLYYADLFSPAFDNLYADLKYYWDQDPPEADLVLMTFGAKPY

SEQ ID NO: 271 - protein, 9 aa, Artificial Sequence
  source: /note=“Description of Artificial Sequence Synthetic peptide”
QKIRKYTMR

SEQ ID NO: 272 - protein, 9 aa, Artificial Sequence
  source: /note=“Description of Artificial Sequence Synthetic peptide”
MALESILRR

SEQ ID NO: 273 - protein, 9 aa, Artificial Sequence
  source: /note=“Description of Artificial Sequence Synthetic peptide”
SPLDSTFYR

SEQ ID NO: 274 - protein, 9 aa, Artificial Sequence
  source: /note=“Description of Artificial Sequence Synthetic peptide”
GVGSPYVSR

SEQ ID NO: 275 - protein, 9 aa, Artificial Sequence
  source: /note=“Description of Artificial Sequence Synthetic peptide”
RILKETELR

SEQ ID NO: 276 - protein, 9 aa, Artificial Sequence
  source: /note=“Description of Artificial Sequence Synthetic peptide”
LACHQLCAR

SEQ ID NO: 277 - protein, 9 aa, Artificial Sequence
  source: /note=“Description of Artificial Sequence Synthetic peptide”
VSEFSRMAR

SEQ ID NO: 278 - protein, 9 aa, Artificial Sequence
  source: /note=“Description of Artificial Sequence Synthetic peptide”
LEDVRLVHR

SEQ ID NO: 279 - protein, 9 aa, Artificial Sequence
  source: /note=“Description of Artificial Sequence Synthetic peptide”
VFQNLQVIR

SEQ ID NO: 280 - protein, 9 aa, Artificial Sequence
  source: /note=“Description of Artificial Sequence Synthetic peptide”
AALCRWGLL

SEQ ID NO: 281 - protein, 9 aa, Artificial Sequence
  source: /note=“Description of Artificial Sequence Synthetic peptide”
QALLHTANR

SEQ ID NO: 282 - protein, 9 aa, Artificial Sequence
  source: /note=“Description of Artificial Sequence Synthetic peptide”
REGPLPAAR

SEQ ID NO: 283 - protein, 9 aa, Artificial Sequence
  source: /note=“Description of Artificial Sequence Synthetic peptide”
TLIDTNRSR

SEQ ID NO: 284 - protein, 9 aa, Artificial Sequence
  source: /note=“Description of Artificial Sequence Synthetic peptide”
RQVPLQRLR

SEQ ID NO: 285 - protein, 310 aa, Artificial Sequence
  source: /note=“Description of Artificial Sequence Synthetic polypeptide”
QALLHTANRAIGRQVPLQRLRADGKQKIRKYTMRADGKGVGSPYVSRRILKETELRADLLEDVRLVHRADGTLIDTNRSRADLGMEHLREVRADGKREGPLPAARRIGMALESILRRPDGKLGISWLGLRADGVKITDFGLARAPLQRLRIVRADGVVFGILIKRRDGKLVHRDLAARATVCAGGCARRDGKIRKYTMRRADGAALCRWGLLADGKKIFGSLAFLPDGKVPIKWMALSDASPLDSTFYRADLVSEFSRMARADLVCVNCSQFLRADLKLACHQLCARADVFQNLQVIRAILSWLGLRSLR

SEQ ID NO: 286 - protein, 9 aa, Artificial Sequence
  source: /note=“Description of Artificial Sequence Synthetic peptide”
RCEKCSKPC

SEQ ID NO: 287 - protein, 9 aa, Artificial Sequence
  source: /note=“Description of Artificial Sequence Synthetic peptide”
SPKANKEIL

SEQ ID NO: 288 - protein, 9 aa, Artificial Sequence
  source: /note=“Description of Artificial Sequence Synthetic peptide”
SPETHLDML

SEQ ID NO: 289 - protein, 9 aa, Artificial Sequence
  source: /note=“Description of Artificial Sequence Synthetic peptide”
PPSPREGPL

SEQ ID NO: 290 - protein, 9 aa, Artificial Sequence
  source: /note=“Description of Artificial Sequence Synthetic peptide”
GAVENPEYL

SEQ ID NO: 291 - protein, 9 aa, Artificial Sequence
  source: /note=“Description of Artificial Sequence Synthetic peptide”
SPGKNGVVK

SEQ ID NO: 292 - protein, 9 aa, Artificial Sequence
  source: /note=“Description of Artificial Sequence Synthetic peptide”
AARPAGATL

SEQ ID NO: 293 - protein, 9 aa, Artificial Sequence
  source: /note=“Description of Artificial Sequence Synthetic peptide”
MPNQAQMRI

SEQ ID NO: 294 - protein, 9 aa, Artificial Sequence
  source: /note=“Description of Artificial Sequence Synthetic peptide”
LPTHDPSPL

SEQ ID NO: 295 - protein, 9 aa, Artificial Sequence
  source: /note=“Description of Artificial Sequence Synthetic peptide”
RKYTMRRLL

SEQ ID NO: 296 - protein, 9 aa, Artificial Sequence
  source: /note=“Description of Artificial Sequence Synthetic peptide”
SPREGPLPA

SEQ ID NO: 297 - protein, 9 aa, Artificial Sequence
  source: /note=“Description of Artificial Sequence Synthetic peptide”
DPASNTAPL

SEQ ID NO: 298 - protein, 9 aa, Artificial Sequence
  source: /note=“Description of Artificial Sequence Synthetic peptide”
LPAARPAGA

SEQ ID NO: 299 - protein, 9 aa, Artificial Sequence
  source: /note=“Description of Artificial Sequence Synthetic peptide”
APQPHPPPA

SEQ ID NO: 300 - protein, 9 aa, Artificial Sequence
  source: /note=“Description of Artificial Sequence Synthetic peptide”
HPPPAFSPA

SEQ ID NO: 301 - protein, 9 aa, Artificial Sequence
  source: /note=“Description of Artificial Sequence Synthetic peptide”
LPTNASLSF

SEQ ID NO: 302 - protein, 9 aa, Artificial Sequence
  source: /note=“Description of Artificial Sequence Synthetic peptide”
VPLQRLRIV

SEQ ID NO: 303 - protein, 9 aa, Artificial Sequence
  source: /note=“Description of Artificial Sequence Synthetic peptide”
LPASPETHL

SEQ ID NO: 304 - protein, 285 aa, Artificial Sequence
  source: /note=“Description of Artificial Sequence Synthetic polypeptide”
AAPRSPLAPSALPAARPAGAPDGALPTHDPSPLAALPASPETHLSDASPETHLDMLAVLDNGDPLASPKANKEILPGAVENPEYLASPGKNGVVKADLPTNASLSFADPASNTAPLAARPAGATLAAPQPHPPPAADGVLQVIRGRILPDGRASPLTSIIADLAPPSPREGPLRDLKHVRENRGRLSDLAHPPPAFSPAPDLKAMPNQAQMRIADLVRKYTMRRLLAGVVKDVFAFADAVPLQRLRIVADGKGSCTLVCPLAIASPREGPLPAADLRCEKCSKPC

SEQ ID NO: 305 - protein, 135 aa, Artificial Sequence
  source: /note=“Description of Artificial Sequence Synthetic polypeptide”
MELAALCRWGLLLALLPPGAPASPKANKEILAARPAGATLALPTHDPSPLAALPASPETHLSDASPETHLDMLADAPPSPREGPLRDLKHVRENRGRLADLACPSGVKPDLADGSTRSGGGDLPIASPLTSIISA

SEQ ID NO: 306 - protein, 9 aa, Artificial Sequence
  source: /note=“Description of Artificial Sequence Synthetic peptide”
VVGILLVVV

SEQ ID NO: 307 - protein, 9 aa, Artificial Sequence
  source: /note=“Description of Artificial Sequence Synthetic peptide”
ILRRRFTHQ

SEQ ID NO: 308 - protein, 9 aa, Artificial Sequence
  source: /note=“Description of Artificial Sequence Synthetic peptide”
VLIQRNPQL

SEQ ID NO: 309 - protein, 9 aa, Artificial Sequence
  source: /note=“Description of Artificial Sequence Synthetic peptide”
QQKIRKYTM

SEQ ID NO: 310 - protein, 9 aa, Artificial Sequence
  source: /note=“Description of Artificial Sequence Synthetic peptide”
FGLARLLDI

SEQ ID NO: 311 - protein, 9 aa, Artificial Sequence
  source: /note=“Description of Artificial Sequence Synthetic peptide”
LGMEHLREV

SEQ ID NO: 312 - protein, 9 aa, Artificial Sequence
  source: /note=“Description of Artificial Sequence Synthetic peptide”
MVHHRHRSS

SEQ ID NO: 313 - protein, 9 aa, Artificial Sequence
  source: /note=“Description of Artificial Sequence Synthetic peptide”
LDSTFYRSL

SEQ ID NO: 314 - protein, 9 aa, Artificial Sequence
  source: /note=“Description of Artificial Sequence Synthetic peptide”
MRILKETEL

SEQ ID NO: 315 - protein, 9 aa, Artificial Sequence
  source: /note=“Description of Artificial Sequence Synthetic peptide”
ECRPRFREL

SEQ ID NO: 316 - protein, 9 aa, Artificial Sequence
  source: /note=“Description of Artificial Sequence Synthetic peptide”
LFRNPHQAL

SEQ ID NO: 317 - protein, 9 aa, Artificial Sequence
  source: /note=“Description of Artificial Sequence Synthetic peptide”
LIKRRQQKI

SEQ ID NO: 318 - protein, 9 aa, Artificial Sequence
  source: /note=“Description of Artificial Sequence Synthetic peptide”
VGILLVVVL

SEQ ID NO: 319 - protein, 330 aa, Artificial Sequence
  source: /note=“Description of Artificial Sequence Synthetic polypeptide”
YISAWPDSLPDLECRPRFRELADVGILLVVVLPDQQKIRKYTMADLFRNPHQALALLIKRRQQKIADLKAYGVTVWELMPDLKLGMEHLREVASPKANKEILALIHHNTHLADIFHKNNQLADMVHHRHRSSADAVPLQRLRIVAILLVVVLGVADVSRLLGICLAFGLARLLDIAILQRLRIVRGADVVGILLVVVPDGKVPIKWMALSLAFLPESFAILQVIRGRILLVVVLGVVFAMRILKETELRTGVLIQRNPQLPDLKILRRRFTHQADLAALCRWGLADLDSTFYRSLRDLRIVRGTQLPIAKISAVVGILLAIMIMVKCWMI

SEQ ID NO: 320 - protein, 157 aa, Artificial Sequence
  source: /note=“Description of Artificial Sequence Synthetic polypeptide”
MELAALCRWGLLLALLPPGAPAIGFHKNNQLALASPKANKEILRDGKDIFHKNNQLPDGKLGMEHLREVADLFRNPHQALALLGCKKIFGSLPDLRIVRGTQLADGVMRILKETELSDGQLRSLTEILADGKECRPRFRELADGQLMPYGCLLPDLK

SEQ ID NO: 321 - protein, 9 aa, Artificial Sequence
  source: /note=“Description of Artificial Sequence Synthetic peptide”
WCMQIAKGM

SEQ ID NO: 322 - protein, 9 aa, Artificial Sequence
  source: /note=“Description of Artificial Sequence Synthetic peptide”
IQRNPQLCY

SEQ ID NO: 323 - protein, 9 aa, Artificial Sequence
  source: /note=“Description of Artificial Sequence Synthetic peptide”
TQCVNCSQF

SEQ ID NO: 324 - protein, 9 aa, Artificial Sequence
  source: /note=“Description of Artificial Sequence Synthetic peptide”
VTSANIQEF

SEQ ID NO: 325 - protein, 9 aa, Artificial Sequence
  source: /note=“Description of Artificial Sequence Synthetic peptide”
VQGNLELTY

SEQ ID NO: 326 - protein, 9 aa, Artificial Sequence
  source: /note=“Description of Artificial Sequence Synthetic peptide”
RACHPCSPM

SEQ ID NO: 327 - protein, 287 aa, Artificial Sequence
  source: /note=“Description of Artificial Sequence Synthetic polypeptide”
LVVVLGVVFAIQRNPQLCYAILVTQCVNCSQFADGTLIDTNRSRASEGAGSDVFALIHHNTHLAIAYGVTVWELMAIGKISWLGLRSLSVKVLGSGAFAQLFEDNYALPLGRELGSGLALASCVTACPYAILVTSANIQEFAIGVQGNLELTYADLTCSPQPEYADLKQVVQGNLELAIGSGAFGTVYRLVMAGVGSPYADGVLQVIRGRILSLAFLPESFADGVWSYGVTVWADARIVRGTQLFWCMQIAKGMADMQIAKGMSYALMTFGAKPYRDLRACHPCSPM

SEQ ID NO: 328 - protein, 9 aa, Artificial Sequence
  source: /note=“Description of Artificial Sequence Synthetic peptide”
SAWPDSLPD

SEQ ID NO: 329 - protein, 9 aa, Artificial Sequence
  source: /note=“Description of Artificial Sequence Synthetic peptide”
LDIDETEYH

SEQ ID NO: 330 - protein, 9 aa, Artificial Sequence
  source: /note=“Description of Artificial Sequence Synthetic peptide”
PEYLTPQGG

SEQ ID NO: 331 - protein, 9 aa, Artificial Sequence
  source: /note=“Description of Artificial Sequence Synthetic peptide”
QRFVVIQNE

SEQ ID NO: 332 - protein, 9 aa, Artificial Sequence
  source: /note=“Description of Artificial Sequence Synthetic peptide”
GDLGMGAAK

SEQ ID NO: 333 - protein, 9 aa, Artificial Sequence
  source: /note=“Description of Artificial Sequence Synthetic peptide”
EEITGYLYI

SEQ ID NO: 334 - protein, 9 aa, Artificial Sequence
  source: /note=“Description of Artificial Sequence Synthetic peptide”
EDLGPASPL

SEQ ID NO: 335 - protein, 217 aa, Artificial Sequence
  source: /note=“Description of Artificial Sequence Synthetic polypeptide”
LRIVRGTQLASEGAGSDVFALDIDETEYHADLKQETELVEPLADARPEYLTPQGGADGVEEITGYLYIPDGKEECRVLQGLADGRELGSGLALAEDLGPASPLATEILKGGVLPLEEITGYLYPLGKAGDLGMGAAKADLELTYLPTNRDGVKVLGSGAFADTELVEPLTPRDLKSAWPDSLPDADDVWSYGVTVADMQIAKGMSYADQRFVVIQNE

SEQ ID NO: 336 - protein, 9 aa, Artificial Sequence
  source: /note=“Description of Artificial Sequence Synthetic peptide”
GRILHNGAY

SEQ ID NO: 337 - protein, 9 aa, Artificial Sequence
  source: /note=“Description of Artificial Sequence Synthetic peptide”
RRLLQETEL

SEQ ID NO: 338 - protein, 9 aa, Artificial Sequence
  source: /note=“Description of Artificial Sequence Synthetic peptide”
ARPAGATLE

SEQ ID NO: 339 - protein, 9 aa, Artificial Sequence
  source: /note=“Description of Artificial Sequence Synthetic peptide”
GRLGSQDLL

SEQ ID NO: 340 - protein, 9 aa, Artificial Sequence
  source: /note=“Description of Artificial Sequence Synthetic peptide”
RRFTHQSDV

SEQ ID NO: 341 - protein, 9 aa, Artificial Sequence
  source: /note=“Description of Artificial Sequence Synthetic peptide”
HRDLAARNV

SEQ ID NO: 342 - protein, 9 aa, Artificial Sequence
  source: /note=“Description of Artificial Sequence Synthetic peptide”
FAGCKKIFG

SEQ ID NO: 343 - protein, 9 aa, Artificial Sequence
  source: /note=“Description of Artificial Sequence Synthetic peptide”
RRQQKIRKY

SEQ ID NO: 344 - protein, 9 aa, Artificial Sequence
  source: /note=“Description of Artificial Sequence Synthetic peptide”
AAGCTGPKH

SEQ ID NO: 345 - protein, 9 aa, Artificial Sequence
  source: /note=“Description of Artificial Sequence Synthetic peptide”
ARVCYGLGM

SEQ ID NO: 346 - protein, 9 aa, Artificial Sequence
  source: /note=“Description of Artificial Sequence Synthetic peptide”
QRASPLTSI

SEQ ID NO: 347 - protein, 9 aa, Artificial Sequence
  source: /note=“Description of Artificial Sequence Synthetic peptide”
CRVLQGLPR

SEQ ID NO: 348 - protein, 9 aa, Artificial Sequence
  source: /note=“Description of Artificial Sequence Synthetic peptide”
AKGLQSLPT

SEQ ID NO: 349 - protein, 9 aa, Artificial Sequence
  source: /note=“Description of Artificial Sequence Synthetic peptide”
RNPQLCYQD

SEQ ID NO: 350 - protein, 9 aa, Artificial Sequence
  source: /note=“Description of Artificial Sequence Synthetic peptide”
HRHRSSSTR

SEQ ID NO: 351 - protein, 342 aa, Artificial Sequence
  source: /note=“Description of Artificial Sequence Synthetic polypeptide”
GRILHNGAYADGCRWGLLLALLQPEQLQVFAILDEAYVMARDAKGLQSLPTADGRLGSQDLLADGRELGSGLALAYLEDVRLVHRDAFAGCKKIFGADGFRNPHQALLPIGKAGEGLACHQLADARPAGATLESLRRLLQETELAAGCTGPKHADAVRGTQLFEDRDLVRKYTMRRLLRDLRIVRGTQLPDLKRNPQLCYQDADLKRQVPLQRLRADAKARVCYGLGMADGVHRDLAARNVPDQRASPLTSIPLLKHRHRSSSTRADLVYLYISAWPDADAKQRFVVIQNEADLVRRQQKIRKYADLKCRVLQGLPRADLYTMRRLLQEADLKRRFTHQSDV

SEQ ID NO: 352 - protein, 9 aa, Artificial Sequence
  source: /note=“Description of Artificial Sequence Synthetic peptide”
LALLPPGAA

SEQ ID NO: 353 - protein, 9 aa, Artificial Sequence
  source: /note=“Description of Artificial Sequence Synthetic peptide”
EPLTPSGAM

SEQ ID NO: 354 - protein, 9 aa, Artificial Sequence
  source: /note=“Description of Artificial Sequence Synthetic peptide”
MPYGCLLDH

SEQ ID NO: 355 - protein, 9 aa, Artificial Sequence
  source: /note=“Description of Artificial Sequence Synthetic peptide”
EILDEAYVM

SEQ ID NO: 356 - protein, 9 aa, Artificial Sequence
  source: /note=“Description of Artificial Sequence Synthetic peptide”
LVTYNTDTF

SEQ ID NO: 357 - protein, 9 aa, Artificial Sequence
  source: /note=“Description of Artificial Sequence Synthetic peptide”
TPTAENPEY

SEQ ID NO: 358 - protein, 9 aa, Artificial Sequence
  source: /note=“Description of Artificial Sequence Synthetic peptide”
DPAPGAGGM

SEQ ID NO: 359 - protein, 9 aa, Artificial Sequence
  source: /note=“Description of Artificial Sequence Synthetic peptide”
FVVIQNEDL

SEQ ID NO: 360 - protein, 9 aa, Artificial Sequence
  source: /note=“Description of Artificial Sequence Synthetic peptide”
RSLLEDDDM

SEQ ID NO: 361 - protein, 9 aa, Artificial Sequence
  source: /note=“Description of Artificial Sequence Synthetic peptide”
NKEILDEAY

SEQ ID NO: 362 - protein, 9 aa, Artificial Sequence
  source: /note=“Description of Artificial Sequence Synthetic peptide”
ECVGEGLAC

SEQ ID NO: 363 - protein, 320 aa, Artificial Sequence
  source: /note=“Description of Artificial Sequence Synthetic polypeptide”
HTVPWDQLFADLVCRWGLLLALRIALDIDETEYHADLARDGDLGMGAARDLPTNASLSFADPASNTAPLALPTHDPSPLADNKEILDEAYADPAPGAGGMAIAEPLTPSGAMAGVVKDVFAFADLTCSPQPEYADLKLVTYNTDTFADLALLPPGAAPDEILDEAYVMPLVVVLGVVFAECVGEGLACATPTAENPEYADRSLLEDDDMALLVFVVIQNEDLALAMPNQAQMRIADLVMSYLEDVRLAILMTFGAKPYADICELHCPALALGKYYWDQDPPEADLSPAFDNLYYADLFSPAFDNLYAILKAMPYGCLLDH

SEQ ID NO: 364 - protein, 149 aa, Artificial Sequence
  source: /note=“Description of Artificial Sequence Synthetic polypeptide”
MELAALCRWGLLLALLPPGAPADGKTPTAENPEYAALPASPETHLPILKYSEDPTVPLPDGALPTHDPSPLADNKEILDEAYADEILDEAYVMPLVVVLGVVFADMQIAKGMSYALMTFGAKPYPLGKAPPPAFSPAFADLHCPALVTY

SEQ ID NO: 365 - protein, 9 aa, Artificial Sequence
  source: /note=“Description of Artificial Sequence Synthetic peptide”
REVRAVTSA

SEQ ID NO: 366 - protein, 9 aa, Artificial Sequence
  source: /note=“Description of Artificial Sequence Synthetic peptide”
SETDGYVAP

SEQ ID NO: 367 - protein, 9 aa, Artificial Sequence
  source: /note=“Description of Artificial Sequence Synthetic peptide”
AENPEYLGL

SEQ ID NO: 368 - protein, 9 aa, Artificial Sequence
  source: /note=“Description of Artificial Sequence Synthetic peptide”
RDLAARNVL

SEQ ID NO: 369 - protein, 9 aa, Artificial Sequence
  source: /note=“Description of Artificial Sequence Synthetic peptide”
LEDDDMGDL

SEQ ID NO: 370 - protein, 9 aa, Artificial Sequence
  source: /note=“Description of Artificial Sequence Synthetic peptide”
EEEAPRSPL

SEQ ID NO: 371 - protein, 9 aa, Artificial Sequence
  source: /note=“Description of Artificial Sequence Synthetic peptide”
QEFAGCKKI

SEQ ID NO: 372 - protein, 9 aa, Artificial Sequence
  source: /note=“Description of Artificial Sequence Synthetic peptide”
EQLQVFETL

SEQ ID NO: 373 - protein, 9 aa, Artificial Sequence
  source: /note=“Description of Artificial Sequence Synthetic peptide”
GERLPQPPI

SEQ ID NO: 374 - protein, 246 aa, Artificial Sequence
  source: /note=“Description of Artificial Sequence Synthetic polypeptide”
MELAALCRWRDLAARNVLPDAQETELVEPLAEEEAPRSPLPDGKEECRVLQGLADAGERLPQPPIADGSETDGYVAPPDAAGEGLACHQLADGRELGSGLALPQLFEDNYALPDALEDDDMGDLPDLKREVRAVTSAASEGAGSDVFATEILKGGVLPLEEITGYLYIPDGKAENPEYLGLPDLKQEVQGYVLIADEQLQVFETLAQVVQGNLELAQEFAGCKKIALCRWGLLLRDAFEDNYALAV

SEQ ID NO: 375 - protein, 9 aa, Artificial Sequence
  source: /note=“Description of Artificial Sequence Synthetic peptide”
CQSLTRTVC

SEQ ID NO: 376 - protein, 9 aa, Artificial Sequence
  source: /note=“Description of Artificial Sequence Synthetic peptide”
RELQLRSLT

SEQ ID NO: 377 - protein, 9 aa, Artificial Sequence
  source: /note=“Description of Artificial Sequence Synthetic peptide”
TRTVCAGGC

SEQ ID NO: 378 - protein, 9 aa, Artificial Sequence
  source: /note=“Description of Artificial Sequence Synthetic peptide”
TDMKLRLPA

SEQ ID NO: 379 - protein, 9 aa, Artificial Sequence
  source: /note=“Description of Artificial Sequence Synthetic peptide”
GENVKIPVA

SEQ ID NO: 380 - protein, 9 aa, Artificial Sequence
  source: /note=“Description of Artificial Sequence Synthetic peptide”
TELRKVKVL

SEQ ID NO: 381 - protein, 9 aa, Artificial Sequence
  source: /note=“Description of Artificial Sequence Synthetic peptide”
GSRCWGESS

SEQ ID NO: 382 - protein, 9 aa, Artificial Sequence
  source: /note=“Description of Artificial Sequence Synthetic peptide”
TGTDMKLRL

SEQ ID NO: 383 - protein, 9 aa, Artificial Sequence
  source: /note=“Description of Artificial Sequence Synthetic peptide”
REYVNARHC

SEQ ID NO: 384 - protein, 340 aa, Artificial Sequence
  source: /note=“Description of Artificial Sequence Synthetic polypeptide”
ISWLGLRSLAEEEAPRSPLRDLAARNVLRLGGENVKIPVARLGKHSDCLACLAIGGERLPQPPIADLTGTDMKLRLPDGKAENPEYLGLADGRELGSGLALREVRAVTSAADGREYVNARHCAQEFAGCKKIAQETELVEPLATELRKVKVLTDMKLRLPAADLKQEVQGYVLIPDLARGGSRCWGESSALGVKITDFGLARATDFGLARLLPDARKYTMRRLLADGRELQLRSLTADLKLDSTFYRSLMELAALCRWATLQGLGISWADLCQSLTRTVCALLHYKDPPFCVAIGYISAWPDSLADCRWGLLLALRDLTRTVCAGGCADLKTFYRSLLED

SEQ ID NO: 385 - protein, 9 aa, Artificial Sequence
  source: /note=“Description of Artificial Sequence Synthetic peptide”
ERGAPPSTF

SEQ ID NO: 386 - protein, 9 aa, Artificial Sequence
  source: /note=“Description of Artificial Sequence Synthetic peptide”
IDSECRPRF

SEQ ID NO: 387 - protein, 9 aa, Artificial Sequence
  source: /note=“Description of Artificial Sequence Synthetic peptide”
GGGDLTLGL

SEQ ID NO: 388 - protein, 9 aa, Artificial Sequence
  source: /note=“Description of Artificial Sequence Synthetic peptide”
PEADQCVAC

SEQ ID NO: 389 - protein, 182 aa, Artificial Sequence
  source: /note=“Description of Artificial Sequence Synthetic polypeptide”
TRTVCAGGCADGGGGDLTLGLARPEADQCVACATLQGLGISWAIAFDGDLGMGAPDAKARGDLTLGLEPPDGKIDSECRPRFADGVKVLGSGAFADGQETELVEPLADGRELGSGLALAQEVQGYVLIALGERGAPPSTFAQEFAGCKKIMELAALCRWALGVKIPVAIKVALLHCPALVTY

SEQ ID NO: 390 - protein, 9 aa, Artificial Sequence
  source: /note=“Description of Artificial Sequence Synthetic peptide”
PDLSVFQNL

SEQ ID NO: 391 - protein, 210 aa, Artificial Sequence
  source: /note=“Description of Artificial Sequence Synthetic polypeptide”
LRIVRGTQLPIAAGGGDLTLGLARPEADQCVACAIAFDGDLGMGAPDAKARGDLTLGLEPPDLKQETELVEPLPIVKVLGSGAFASEGAGSDVFPDGRELGSGLALAQEVQGYVLIADGKEECRVLQGLPDLKLEEITGYLYATEILKGGVLPLEEITGYLYIADMELAALCRWADARPDLSVFQNLADLTDFGLARLLPDTRTVCAGGC

SEQ ID NO: 392 - protein, 9 aa, Artificial Sequence
  source: /note=“Description of Artificial Sequence Synthetic peptide”
PEGRYTFGA

SEQ ID NO: 393 - protein, 9 aa, Artificial Sequence
  source: /note=“Description of Artificial Sequence Synthetic peptide”
MEHLREVRA

SEQ ID NO: 394 - protein, 9 aa, Artificial Sequence
  source: /note=“Description of Artificial Sequence Synthetic peptide”
LGMGAAKGL

SEQ ID NO: 395 - protein, 9 aa, Artificial Sequence
  source: /note=“Description of Artificial Sequence Synthetic peptide”
TSANIQEFA

SEQ ID NO: 396 - protein, 9 aa, Artificial Sequence
  source: /note=“Description of Artificial Sequence Synthetic peptide”
LPSETDGYV

SEQ ID NO: 397 - protein, 9 aa, Artificial Sequence
  source: /note=“Description of Artificial Sequence Synthetic peptide”
AEQRASPLT

SEQ ID NO: 398 - protein, 9 aa, Artificial Sequence
  source: /note=“Description of Artificial Sequence Synthetic peptide”
INCTHSCVD

SEQ ID NO: 399 - protein, 9 aa, Artificial Sequence
  source: /note=“Description of Artificial Sequence Synthetic peptide”
EEAPRSPLA

SEQ ID NO: 400 - protein, 9 aa, Artificial Sequence
  source: /note=“Description of Artificial Sequence Synthetic peptide”
CELHCPALV

SEQ ID NO: 401 - protein, 9 aa, Artificial Sequence
  source: /note=“Description of Artificial Sequence Synthetic peptide”
KEILDEAYV

SEQ ID NO: 402 - protein, 9 aa, Artificial Sequence
  source: /note=“Description of Artificial Sequence Synthetic peptide”
CEKCSKPCA

SEQ ID NO: 403 - protein, 340 aa, Artificial Sequence
  source: /note=“Description of Artificial Sequence Synthetic polypeptide”
CELHCPALVADGGENVKIPVAALPASPETHLRDARPEGRYTFGAADGKIDSECRPRFADLKGERLPQPPIAILAEEAPRSPLAADGAEEITGYLYIALPAARPAGAPDGKMEHLREVRAPDGRELQLRSLTADLKKEILDEAYVATAFDGDLGMGAPDLKREVRAVTSAALPSETDGYVADGAEQRASPLTADGAGEGLACHQLADGRELGSGLALADCEKCSKPCAADGVQEVQGYVLIADLTSANIQEFAADLDSTFYRSLMELAALCRWATGKAINCTHSCVDRDAFEDNYALAVRDLGMGAAKGLVSRLLGICLPDVKIPVAIKVAIASCVTACPY

SEQ ID NO: 404 - protein, 9 aa, Artificial Sequence
  source: /note=“Description of Artificial Sequence Synthetic peptide”
LPQPPICTI

SEQ ID NO: 405 - protein, 9 aa, Artificial Sequence
  source: /note=“Description of Artificial Sequence Synthetic peptide”
ENVKIPVAI

SEQ ID NO: 406 - protein, 222 aa, Artificial Sequence
  source: /note=“Description of Artificial Sequence Synthetic polypeptide”
CRWGLLLALPDENVKIPVAIAYGVTVWELMAALPASPETHLARPDLSVFQNLPDLPTNASLSFADGALPTHDPSPLPDLALPSETDGYVPDLKLGMEHLREVADLPQPPICTIADGVQEVQGYVLIADEQLQVFETLALGMGAAKGLPDKGMSYLEDVAQEFAGCKKISVGILLVVVLAMPNQAQMRIADLKLQLRSLTEIADVKIPVAIKVATDFGLARLL

SEQ ID NO: 407 - protein, 9 aa, Artificial Sequence
  source: /note=“Description of Artificial Sequence Synthetic peptide”
DDMGDLVDA

SEQ ID NO: 408 - protein, 9 aa, Artificial Sequence
  source: /note=“Description of Artificial Sequence Synthetic peptide”
RPEDECVGE

SEQ ID NO: 409 - protein, 9 aa, Artificial Sequence
  source: /note=“Description of Artificial Sequence Synthetic peptide”
SPQPEYVNQ

SEQ ID NO: 410 - protein, 9 aa, Artificial Sequence
  source: /note=“Description of Artificial Sequence Synthetic peptide”
VENPEYLTP

SEQ ID NO: 411 - protein, 9 aa, Artificial Sequence
  source: /note=“Description of Artificial Sequence Synthetic peptide”
AGVGSPYVS

SEQ ID NO: 412 - protein, 9 aa, Artificial Sequence
  source: /note=“Description of Artificial Sequence Synthetic peptide”
SPLTSIISA

SEQ ID NO: 413 - protein, 9 aa, Artificial Sequence
  source: /note=“Description of Artificial Sequence Synthetic peptide”
GPLPAARPA

SEQ ID NO: 414 - protein, 9 aa, Artificial Sequence
  source: /note=“Description of Artificial Sequence Synthetic peptide”
CPSGVKPDL

SEQ ID NO: 415 - protein, 274 aa, Artificial Sequence
  source: /note=“Description of Artificial Sequence Synthetic polypeptide”
ASPLDSTFYRADGVENPEYLTPAALPASPETHLARAGVGSPYVSRDLPTNASLSFADGALPTHDPSPLADLLERPKTLSPALAFDGDLGMGAPDAKARGDLTLGLEPPDLARDDMGDLVDAPDLARPEDECVGEATPTAENPEYALAMPNQAQMRIADLKLPQPPICTIADASPLTSIISAADCRWGLLLALAGPLPAARPAPDAAPRSPLAPSALAASPQPEYVNQALGVKIPVAIKVADACPSGVKPDLADLHCPALVTYSDASPAFDNLYY

SEQ ID NO: 416 - protein, 9 aa, Artificial Sequence
  source: /note=“Description of Artificial Sequence Synthetic peptide”
LVEPLTPSG

SEQ ID NO: 417 - protein, 9 aa, Artificial Sequence
  source: /note=“Description of Artificial Sequence Synthetic peptide”
LTSIISAVV

SEQ ID NO: 418 - protein, 9 aa, Artificial Sequence
  source: /note=“Description of Artificial Sequence Synthetic peptide”
RKVKVLGSG

SEQ ID NO: 419 - protein, 9 aa, Artificial Sequence
  source: /note=“Description of Artificial Sequence Synthetic peptide”
SPLAPSEGA

SEQ ID NO: 420 - protein, 9 aa, Artificial Sequence
  source: /note=“Description of Artificial Sequence Synthetic peptide”
RGRILHNGA

SEQ ID NO: 421 - protein, 9 aa, Artificial Sequence
  source: /note=“Description of Artificial Sequence Synthetic peptide”
CPALVTYNT

SEQ ID NO: 422 - protein, 9 aa, Artificial Sequence
  source: /note=“Description of Artificial Sequence Synthetic peptide”
MPIWKFPDE

SEQ ID NO: 423 - protein, 9 aa, Artificial Sequence
  source: /note=“Description of Artificial Sequence Synthetic peptide”
WKDIFHKNN

SEQ ID NO: 424 - protein, 9 aa, Artificial Sequence
  source: /note=“Description of Artificial Sequence Synthetic peptide”
LRENTSPKA

SEQ ID NO: 425 - protein, 287 aa, Artificial Sequence
  source: /note=“Description of Artificial Sequence Synthetic polypeptide”
AWKDIFHKNNADAFDGDLGMGAPDLKREVRAVTSAALLAEEAPRSPLAADGARDGDPASNTAALPAARPAGAAIWIPDGENVSDLRENTSPKARDLVEPLTPSGADGLTSIISAVVARKVKVLGSGADGVRELQLRSLTADLKLPQPPICTIADLQRLRIVRGPDLKRGRILHNGAADASPLTSIISAASPLAPSEGAACPALVTYNTADAVPLQRLRIVADAAAMPNQAQMRIADLKAYKDPPFCVARDLAMPIWKFPDEADGAMPYGCLLDHADGKWGLLLALLP

SEQ ID NO: 426 - protein, 9 aa, Artificial Sequence
  source: /note=“Description of Artificial Sequence Synthetic peptide”
ATLERPKTL

SEQ ID NO: 427 - protein, 9 aa, Artificial Sequence
  source: /note=“Description of Artificial Sequence Synthetic peptide”
PAFDNLYYW

SEQ ID NO: 428 - protein, 231 aa, Artificial Sequence
  source: /note=“Description of Artificial Sequence Synthetic polypeptide”
MELAALCRWAVTSANIQEFALGKENVKIPVAIADGKDIFHKNNQLRDATLERPKTLLVVVLGVVFPTLQGLGISWADVFDGDLGMRDLVALCRWGLLLPDGKISWLGLRSLRSLLEDDDMADGGSGAFGTVYADAGTQLFEDNYRDLKLSYMPIWKFADLKPAFDNLYYWADLQLMPYGCLLPDLKMSYLEDVRLRDVWSYGVTVPDLKRFTHQSDVWADLVHTVPWDQLF

SEQ ID NO: 429 - protein, 9 aa, Artificial Sequence
  source: /note=“Description of Artificial Sequence Synthetic peptide”
RSGGGDLTL

SEQ ID NO: 430 - protein, 9 aa, Artificial Sequence
  source: /note=“Description of Artificial Sequence Synthetic peptide”
KGCPAEQRA

SEQ ID NO: 431 - protein, 9 aa, Artificial Sequence
  source: /note=“Description of Artificial Sequence Synthetic peptide”
LCYQDTILW

SEQ ID NO: 432 - protein, 9 aa, Artificial Sequence
  source: /note=“Description of Artificial Sequence Synthetic peptide”
GAMPNQAQM

SEQ ID NO: 433 - protein, 9 aa, Artificial Sequence
  source: /note=“Description of Artificial Sequence Synthetic peptide”
VTGASPGGL

SEQ ID NO: 434 - protein, 9 aa, Artificial Sequence
  source: /note=“Description of Artificial Sequence Synthetic peptide”
LGSQDLLNW

SEQ ID NO: 435 - protein, 9 aa, Artificial Sequence
  source: /note=“Description of Artificial Sequence Synthetic peptide”
RGQECVEEC

SEQ ID NO: 436 - protein, 284 aa, Artificial Sequence
  source: /note=“Description of Artificial Sequence Synthetic polypeptide”
PAFDNLYYWAILCTIDVYMIMADLVRMARDPQRFADKGCPAEQRAPDLKLGSQDLLNWAIISAVVGILALRCEKCSKPCAILVTSANIQEFADLGAMPNQAQMADAVTGASPGGLPISAVVGILLPDRSGGGDLTLAYLSTDVGSCALAALCRWGLALASCVTACPYADLHTVPWDQLFADLKLSYMPIWKFADGRASPLTSIIADGVTVWELMTFADGVARGQECVEECADLRIVRGTQLFTRTVCAGGCADKIFGSLAFLPDVCTGTDMKLADLCYQDTILW

SEQ ID NO: 437 - protein, 9 aa, Artificial Sequence
  source: /note=“Description of Artificial Sequence Synthetic peptide”
LRHLYQGCQ

SEQ ID NO: 438 - protein, 18 aa, Artificial Sequence
  source: /note=“Description of Artificial Sequence Synthetic peptide”
CLHFNHSGICELHCPALV

SEQ ID NO: 439 - protein, 9 aa, Artificial Sequence
  source: /note=“Description of Artificial Sequence Synthetic peptide”
LQVFETLEE

SEQ ID NO: 440 - protein, 9 aa, Artificial Sequence
  source: /note=“Description of Artificial Sequence Synthetic peptide”
LRSLRELGS

SEQ ID NO: 441 - protein, 11 aa, Artificial Sequence
  source: /note=“Description of Artificial Sequence Synthetic peptide”
LCFVHTVPWDQ

SEQ ID NO: 442 - protein, 9 aa, Artificial Sequence
  source: /note=“Description of Artificial Sequence Synthetic peptide”
LRGQECVEE

SEQ ID NO: 443 - protein, 9 aa, Artificial Sequence
  source: /note=“Description of Artificial Sequence Synthetic peptide”
CPINCTHSC

SEQ ID NO: 444 - protein, 9 aa, Artificial Sequence
  source: /note=“Description of Artificial Sequence Synthetic peptide”
IRKYTMRRL

SEQ ID NO: 445 - protein, 17 aa, Artificial Sequence
  source: /note=“Description of Artificial Sequence Synthetic peptide”
MRILKETELRKVKVLGS

SEQ ID NO: 446 - protein, 17 aa, Artificial Sequence
  source: /note=“Description of Artificial Sequence Synthetic peptide”
VKIPVAIKVLRENTSPK

SEQ ID NO: 447 - protein, 26 aa, Artificial Sequence
  source: /note=“Description of Artificial Sequence Synthetic peptide”
YVMAGVGSPYVSRLLGICLTSTVQLV

SEQ ID NO: 448 - protein, 9 aa, Artificial Sequence
  source: /note=“Description of Artificial Sequence Synthetic peptide”
VRLVHRDLA

SEQ ID NO: 449 - protein, 15 aa, Artificial Sequence
  source: /note=“Description of Artificial Sequence Synthetic peptide”
FGLARLLDIDETEYH

SEQ ID NO: 450 - protein, 16 aa, Artificial Sequence
  source: /note=“Description of Artificial Sequence Synthetic peptide”
WMALESILRRRFTHQS

SEQ ID NO: 451 - protein, 15 aa, Artificial Sequence
  source: /note=“Description of Artificial Sequence Synthetic peptide”
CTIDVYMIMVKCWMI

SEQ ID NO: 452 - protein, 13 aa, Artificial Sequence
  source: /note=“Description of Artificial Sequence Synthetic peptide”
CRPRFRELVSEFS

SEQ ID NO: 453 - protein, 141 aa, Artificial Sequence
  source: /note=“Description of Artificial Sequence Synthetic polypeptide”
AKFVAAWTLKAAAKKAVVGILLVVVLGVVFGILIKRRQQKIRKKPICTIDVYMIMVKCWMIDSEKKAQMRILKETELRKVKVLGSGAKKIKWMALESILRRRFTHQSDVKKPICTIDVYMIMVKCWMIDSRKRSHAGYQTI

SEQ ID NO: 454 - protein, 296 aa, Artificial Sequence
  source: /note=“Description of Artificial Sequence Synthetic polypeptide”
MHRRRSRSCREDQKPVMDDQRDLISNNEQLPMLGRRPGAPESKCSRGALYTGFSILVTLLLAGQATTAYFLYQQQGRLDKLTVTSQNLQLENLRMKLPKPPKPVSKMRMATPLLMQALPMGALPQGPMQNATKYGNMTEDHVMHLLQNADPLKVYPPLKGSFPENLRHLKNTMETIDWKVFESWMHHWLLFEMSRHSLEQKPTDAPPKVLTKCQEEVSHIPAVHPGSFRPKCDENGNYLPLQCYGSIGYCWCVFPNGTEVPNTRSRGHHNCSESLELEDPSSGLGVTKQDLGPVPM

SEQ ID NO: 455 - protein, 76 aa, Artificial Sequence
  source: /note=“Description of Artificial Sequence Synthetic polypeptide”
MQIFVKTLTGKTITLEVEPSDTIENVKAKIQDKEGIPPDQQRLIFAGKQLEDGRTLSDYNIQKESTLHLVLRLRGV

SEQ ID NO: 456 - protein, 602 aa, Artificial Sequence
  source: /note=“Description of Artificial Sequence Synthetic polypeptide”
MELAALCRWGLLLALLPPGAPDGENVKIPVAIKVLRENTADGKEECRVLQGLPDGKYSEDPTVPLPDDEAYVMAGVADLKQETELVEPLTPPDGRASPLTSIISAVVGILLVVVLGVVFPDAGMEHLREVRADGKDIFHKNNQLPDLQPEQLQVFRDAQEVQGYVLIPDLAFDGDLGMGAPDLQVIRGRILPDVKVLGSGAFGTVYPIGDLTLGLEPPDLKASCVTACPYATLQGLGISWLGLRSLRELGSGLALPMQIAKGMSYALFGPEADQCVPDLKLSYMPIWKFADLKPLQRLRIVRGTQLFEDNYALAVARGAPPSTFKAGVVKDVFAFRDLVKITDFGLARLLPLVHRDLAARADVWSYGVTVRDTTPVTGASPRDLYISAWPDSLRTVCAGGCARSDKIFGSLAFLPDLHCPALVTYADDSTFYRSLLADGKQLMPYGCLLADGGSCTLVCPLAKFVAAWTLKAAAKKAVVGILLVVVLGVVFGILIKRRQQKIRKKPICTIDVYMIMVKCWMIDSEKKAQMRILKETELRKVKVLGSGAKKIKWMALESILRRRFTHQSDVKKPICTIDVYMIMVKCWMIDSRKRSHAGYQTI

SEQ ID NO: 457 - protein, 309 aa, Artificial Sequence
  source: /note=“Description of Artificial Sequence Synthetic polypeptide”
MELAALCRWGLLLALLPPGAPPDLLALLPPGAPDATLEEITGYLAILDEAYVMAPILHNGAYSLPQLFEDNYALSIISAVVGIAQLMPYGCLLRLLVVVLGVVRDLQLRSLTEIAILLVVVLGVPDAVVGILLVVADALCRWGLLLADYISAWPDSLRDKIFGSLAFLAKFVAAWTLKAAAKKAVVGILLVVVLGVVFGILIKRRQQKIRKKPICTIDVYMIMVKCWMIDSEKKAQMRILKETELRKVKVLGSGAKKIKWMALESILRRRFTHQSDVKKPICTIDVYMIMVKCWMIDSRKRSHAGYQTI

SEQ ID NO: 458 - protein, 290 aa, Artificial Sequence
  source: /note=“Description of Artificial Sequence Synthetic polypeptide”
MELAALCRWGLLLALLPPGAPADGKTPTAENPEYAALPASPETHLPILKYSEDPTVPLPDGALPTHDPSPLADNKEILDEAYADEILDEAYVMPLVVVLGVVFADMQIAKGMSYALMTFGAKPYPLGKAPPPAFSPAFADLHCPALVTYAKFVAAWTLKAAAKKAVVGILLVVVLGVVFGILIKRRQQKIRKKPICTIDVYMIMVKCWMIDSEKKAQMRILKETELRKVKVLGSGAKKIKWMALESILRRRFTHQSDVKKPICTIDVYMIMVKCWMIDSRKRSHAGYQTI

SEQ ID NO: 459 - protein, 330 aa, Artificial Sequence
  source: /note=“Description of Artificial Sequence Synthetic polypeptide”
DQICIGYHANNSTEQVDTIMEKNVTVTHAQDILEKTHNGKLCDLDGVKPLILRDCSVAGWLLGNPMCDEFINVPEWSYIVEKANPTNDLCYPGSFNDYEELKHLLSRINHFEKIQIIPKSSWSDHEASSGVSSACPYLGSPSFFRNVVWLIKKNSTYPTIKKSYNNTNQEDLLVLWGIHHPNDAAEQTRLYQNPTTYISIGTSTLNQRLVPKIATRSKVNGQSGRMEFFWAILKPNDAINFESNGNFIAPEYAYKIVKKGDSAIMKSELEYGNCNTKCQTPMGAINSSMPFHNIHPLTIGECPKYVKSNRLVLATGLRNSPQRESRRKKR

SEQ ID NO: 460 - protein, 4 aa, Artificial Sequence
  source: /note=“Description of Artificial Sequence Synthetic peptide”
ADAA

SEQ ID NO: 461 - protein, 6 aa, Artificial Sequence
  source: /note=“Description of Artificial Sequence Synthetic peptide”
ADLVAG

SEQ ID NO: 462 - protein, 6 aa, Artificial Sequence
  source: /note=“Description of Artificial Sequence Synthetic peptide”
ADLAVK

SEQ ID NO: 463 - protein, 6 aa, Artificial Sequence
  source: /note=“Description of Artificial Sequence Synthetic peptide”
AAAAAA

SEQ ID NO: 464 - protein, 3 aa, Artificial Sequence
  source: /note=“Description of Artificial Sequence Synthetic peptide”
ARY